(19) United States
(12) Patent Application Publication
(10) Pub. No.: US 2023/0241024 A1
(43) Pub. Date: Aug. 3, 2023

(54) METHODS OF TREATING METABOLIC DISORDERS

(71) Applicant: Sunovion Pharmaceuticals Inc., Marlborough, MA (US)

(72) Inventors: Linda Jane BRISTOW, Boston, MA (US); Nina DEDIC, Somerville, MA (US); Eva HAJOS-KORCSOK, Boston, MA (US); Seth Cabot HOPKINS, Northborough, MA (US); Philip Glyn JONES, Danvers, MA (US); Kenneth S. KOBLAN, Sudbury, MA (US); Snezana MILANOVIC, Cambridge, MA (US); Colleen Marie SYNAN, Townsend, MA (US); Kuangnan XIONG, Southborough, MA (US)

(73) Assignee: Sunovion Pharmaceuticals Inc., Marlborough, MA (US)

(21) Appl. No.: 17/933,881

(22) Filed: Sep. 21, 2022

Related U.S. Application Data
(60) Provisional application No. 63/362,003, filed on Mar. 28, 2022.
(60) Provisional application No. 63/261,515, filed on Sep. 23, 2021.

(51) Int. Cl. A61K 31/381; A61P 3/04; A61P 3/10; A61P 25/18; A61K 31/5513

(52) U.S. Cl. CPC A61K 31/381; A61K 31/5513; A61P 3/04; A61P 3/10; A61P 25/18

(57) ABSTRACT

Provided herein are compounds, pharmaceutical compositions, and methods of use thereof, including methods of treating metabolic disorders and psychiatric disorders and diseases associated with metabolic disorders. For example, provided herein is Compound 1:

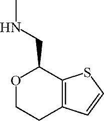

or a pharmaceutically acceptable salt thereof, and pharmaceutical compositions and methods of use thereof, including methods of treating metabolic disorders and psychiatric disorders associated with metabolic disorders.

### CROSS REFERENCE TO RELATED APPLICATIONS

This application claims priority to U.S. Provisional Application Nos. U.S.S.N. 63/261,515, filed Sep. 23, 2021, and U.S.S.N. 63/362,003, filed Mar. 28, 2022, the entire contents of which are hereby incorporated by reference as if fully set forth herein.

### FIELD

The present disclosure relates to compounds, pharmaceutical compositions, and methods of use thereof, including methods of treating a metabolic disorder and methods of treating psychiatric disorders and diseases associated with a metabolic disorder.

### BACKGROUND

Metabolic disorders are an increasingly prevalent category of disorders including Obesity [including obesity comorbidities including but not limited to metabolic syndrome, dyslipidemia, Type III dyslipidemia, hypertension, insulin resistance, diabetes (including Type 1 and Type 2 diabetes), coronary artery disease, and heart failure]; Overweightness or increased weight; Increased body mass index; Metabolic syndrome; Diabetes [including diabetes-related disorders including Type 1 diabetes (insulin-dependent diabetes mellitus, IDDM) and Type 2 diabetes (non-insulin-dependent diabetes mellitus, NIDDM)]; Diabetic complications (including, but not limited to atherosclerosis, coronary heart disease, stroke, peripheral vascular disease, nephropathy, hypertension, neuropathy and nephropathy); Impaired glucose tolerance; Elevated blood glucose; Insulin resistance; Insulin insensitivity; Hyperglycemia; Fatty liver disease; Non-alcoholic fatty liver disease; Hepatic insulin resistance; Glycosuria; Increased blood triglycerides; Increased appetite; or Dyslipidemia. Metabolic syndrome alone affects approximately 25% of North Americans. Mottillo et al. JOURNAL OF THE AMERICAN COLLEGE OF CARDIOLOGY 54(14), 1113-32 (2010). A variety of factors can play a role in development of a metabolic disorder. Pharmaceutical treatments for certain neurological or psychiatric diseases and disorders (e.g., antipsychotics for treating schizophrenia) have been associated with metabolic disorders such as weight gain, lipid disturbance, and glucose dysregulation. Pillinger et al. LANCET PSYCHIATRY 7, 64-77 (2020). Accordingly, there is a need for novel therapeutic agents for treating metabolic disorders, including treating metabolic disorders associated with neurological and psychiatric diseases and disorders and their treatment.

### SUMMARY

Provided herein are the methods of treating metabolic disorders, including those caused by treatment with typical and atypical antipsychotics, using Compound 1:

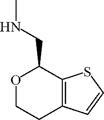

or a pharmaceutically acceptable salt thereof. In some embodiments, the metabolic disorders are associated with a neurological or psychiatric disease or disorder. In some embodiments, the neurological or psychiatric disease or disorder is schizophrenia. In some embodiments, the neurological or psychiatric disease or disorder is adjunctive major depressive disorder (aMDD).

In some embodiments, the neurological or psychiatric disease or disorder is generalized anxiety disorder (GAD).

In an embodiment, the disclosure relates to a method of treating schizophrenia in a human subject in need thereof comprising:
(a) administering to said subject a therapeutically effective amount of Compound 1 at the subject’s bedtime (“evening dose”); and
(b) administering to said subject a therapeutically effective amount of Compound 1 in the morning (“morning dose”).

In an embodiment, the disclosure relates to a method of treating schizophrenia in a human subject in need thereof comprising:
(a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and
(b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL.

In an embodiment, the disclosure relates to a method of treating schizophrenia with weight reduction in a human subject in need thereof comprising:
(a) administering to said subject a therapeutically effective amount of Compound 1 at the subject’s bedtime (“evening dose”); and
(b) administering to said subject a therapeutically effective amount of Compound 1 in the morning (“morning dose”),
and the subject’s weight is reduced after about 4-52 weeks of Compound 1 administration.

In an embodiment, the disclosure relates to a method of treating schizophrenia with weight reduction in a human subject in need thereof comprising:
(a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and
(b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL,
and the subject’s weight is reduced after about 4-52 weeks of Compound 1 administration.

In an embodiment, the disclosure relates to a method of treating schizophrenia and improving insulin secretion efficiency in a type 2 diabetic human subject in need thereof comprising:
(a) administering to said subject a therapeutically effective amount of Compound 1 at the subject’s bedtime (“evening dose”); and
(b) administering to said subject a therapeutically effective amount of Compound 1 in the morning (“morning dose”),
and improving the subject’s insulin secretion efficiency.

In an embodiment, the disclosure relates to a method of treating schizophrenia and improving insulin secretion efficiency in a type 2 diabetic human subject in need thereof comprising:
(a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and
(b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL,
and improving the subject’s insulin secretion efficiency.

In an embodiment, the disclosure relates to a method of treating adjunctive major depressive disorder (aMDD) in a human subject in need thereof comprising:
(a) administering to said subject a therapeutically effective amount of Compound 1 at the subject’s bedtime (“evening dose”); and
(b) administering to said subject a therapeutically effective amount of Compound 1 in the morning (“morning dose”).

In an embodiment, the disclosure relates to a method of treating adjunctive major depressive disorder (aMDD) in a human subject in need thereof comprising:
(a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and
(b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL.

In an embodiment, the disclosure relates to a method of treating generalized anxiety disorder (GAD) in a human subject in need thereof comprising:
(a) administering to said subject a therapeutically effective amount of Compound 1 at the subject’s bedtime (“evening dose”); and
(b) administering to said subject a therapeutically effective amount of Compound 1 in the morning (“morning dose”).

In an embodiment, the disclosure relates to a method of treating generalized anxiety disorder (GAD) in a human subject in need thereof comprising:
(a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and
(b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL.

In an embodiment, the disclosure relates to a method of reducing weight in a human subject in need thereof comprising:
(a) administering to said subject a therapeutically effective amount of Compound 1 at the subject’s bedtime (“evening dose”); and
(b) administering to said subject a therapeutically effective amount of Compound 1 in the morning (“morning dose”),
and the subject’s weight is reduced after about 4-52 weeks of Compound 1 administration.

In an embodiment, the disclosure relates to a method of reducing weight in a human in need thereof subject comprising:
(a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and
(b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL,
wherein, the subject’s weight is reduced after about 4-52 weeks of Compound 1 administration.

In an embodiment, the disclosure relates to a method of treating type 2 diabetes by increasing insulin secretion efficiency in a human subject in need thereof comprising:
(a) administering to said subject a therapeutically effective amount of Compound 1 at the subject’s bedtime (“evening dose”); and
(b) administering to said subject a therapeutically effective amount of Compound 1 in the morning (“morning dose”),
and improving the subject’s insulin secretion efficiency.

In an embodiment, the disclosure relates to a method of treating type 2 diabetes by increasing insulin secretion efficiency in a human subject in need thereof comprising:
(a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and
(b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL,
and improving the subject’s insulin secretion efficiency.

In one embodiment, this disclosure provides a method of treating schizophrenia, depression, or anxiety in a human subject in need thereof comprising:
a) administering to the subject a therapeutically effective amount of Compound 1 at the subject’s bedtime (“evening dose”); and
b) administering to the subject a therapeutically effective amount of Compound 1 in the morning (“morning dose”).

In another embodiment, this disclosure provides a method of treating schizophrenia, depression, or anxiety with weight reduction in a human subject in need thereof comprising:
a) administering to the subject a therapeutically effective amount of Compound 1 at the subject’s bedtime (“evening dose”); and
b) administering to the subject a therapeutically effective amount of Compound 1 in the morning (“morning dose”),
and the subject’s weight is reduced after about 4-52 weeks of Compound 1 administration.

In another embodiment, this disclosure provides a method of treating schizophrenia, depression, or anxiety and improving insulin secretion efficiency in a type 2 diabetic human subject in need thereof comprising:
a) administering to the subject a therapeutically effective amount of Compound 1 at the subject’s bedtime (“evening dose”); and
b) administering to the subject a therapeutically effective amount of Compound 1 in the morning (“morning dose”),
and improving the subject’s insulin secretion efficiency.

In another embodiment, the disclosure provides a method of treating schizophrenia, depression, or anxiety in a human subject in need thereof having an excess body mass index (BMI) defined by a BMI ≥ 25 comprising administering to the subject a therapeutically effective amount of Compound 1.

Another embodiment provides a method of treating schizophrenia, depression, or anxiety in a human subject in need thereof having diabetes or metabolic syndrome comprising administering to the subject a therapeutically effective amount of Compound 1.

One embodiment provides a method of treating schizophrenia, depression, or anxiety in a human subject in need thereof having a metabolic disorder selected from obesity; overweightness or increased weight; increased body mass index; metabolic syndrome; diabetes or diabetes-related disorders (type 1 diabetes (insulin-dependent diabetes mellitus, IDDM) and Type 2 diabetes (non-insulin-dependent diabetes mellitus, NIDDM)); diabetic complications; impaired glucose tolerance; elevated blood glucose; insulin resistance; insulin insensitivity; hyperglycemia; fatty liver disease; non-alcoholic fatty liver disease; hepatic insulin resistance; glycosuria; increased blood triglycerides; increased appetite; or dyslipidemia, comprising administering to the subject a therapeutically effective amount of Compound 1.

In one embodiment, the disclosure provides a method of treating schizophrenia, depression, or anxiety in a human subject in need thereof having a metabolic disorder selected from insulin resistance, insulin insensitivity, impaired glucose tolerance, and elevated blood glucose comprising administering to the subject a therapeutically effective amount of Compound 1.

In another embodiment, the disclosure provides a method of treating schizophrenia, depression, or anxiety in a human subject in need thereof, wherein the human subject is further in need of delayed gastric emptying, comprising administering to the subject a therapeutically effective amount of Compound 1.

In another embodiment, the disclosure provides a method of treating schizophrenia, depression, or anxiety in a human subject in need thereof, comprising administering to the subject a therapeutically effective amount of Compound 1 in the fed state.

In an embodiment, the disclosure relates to the methods as described herein, wherein, the pharmaceutically acceptable salt of Compound 1 is a hydrochloride salt. In another embodiment, the hydrochloride salt is crystalline. In the foregoing embodiments, Compound 1 may be administered prior to or with the morning meal and/or the evening does may be administered prior to or with the evening meal.

### BRIEF DESCRIPTION OF THE DRAWINGS

FIG. 1 shows the effect of Compound 1 on various metabolic parameters in a human clinical study.

### DETAILED DESCRIPTION

A description of embodiments follows.

### Definitions

Provided herein are definitions to assist with interpreting this disclosure. Whenever appropriate, terms used in the singular will also include the plural. Unless the context clearly indicates otherwise, terms used herein have the following meanings.

All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the present disclosure otherwise claimed.

Unless otherwise specified, the word “includes” (or any variation thereon, e.g., “include”, “including”, etc.) is intended to be open-ended. For example, “The term “parenteral” as used herein includes subcutaneous, intravenous, intramuscular, intra-articular, intra-synovial, intrasternal, intrathecal, intrahepatic, intralesional and intracranial injection or infusion techniques” means that parenteral includes, but is not limited to, subcutaneous, intravenous, intramuscular, intra-articular, intra-synovial, intrasternal, intrathecal, intrahepatic, intralesional and intracranial injection or infusion techniques.

Where one or more ranges are referred to throughout this specification, each range is understood to encompass each discrete point within the range, including the endpoints describing the range, as if the same were fully set forth herein.

“Antipsychotics” are commonly categorized as “typical” and “atypical” antipsychotics. In general, typical antipsychotic drugs act on the dopaminergic system, blocking the dopamine type 2 (D2) receptors. Atypical antipsychotics have lower affinity and occupancy for the dopaminergic receptors, and a high degree of occupancy of the serotoninergic 5-HT2A receptors. Commonly prescribed typical antipsychotics include: haloperidol, loxapine, thioridazine, molindone, thiothixene, fluphenazine, mesoridazine, trifluoperazine, perphenazine, and chlorpromazine. Commonly prescribed atypical antipsychotics include: aripiprazole, clozapine, ziprasidone, risperidone, quetiapine, and olanzapine.

The N-desmethyl metabolite of Compound 1 has been reported by Chen YL et al., J. Pharm. Biomed. Anal. 2021;207, and has the following chemical structure:

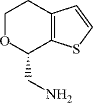 [SMILES: NC[C@@H]1OCCc2ccsc21]

As used herein, the terms “subject” and “patient” are used interchangeably. A “patient” or “subject,” unless otherwise specified, includes both humans and other animals, particularly mammals. Thus, the methods are applicable to both human therapy and veterinary applications. In some embodiments, the patient is a mammal, for example, a primate. In some embodiments, the patient is a human.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms, in light of a diagnosis, and/or in light of genetic or other susceptibility factors). Treatment may also be continued after symptoms have resolved, for example to prevent or delay their recurrence.

The phrase “pharmaceutically acceptable” means that the substance or composition the phrase modifies must be, within the scope of sound medical judgment, suitable for use in contact with the tissues of humans and non-human animals without undue toxicity, irritation, allergic response and the like, and are commensurate with a reasonable benefit/risk ratio. If a substance is part of a composition or formulation, the substance must also be compatible chemically and/or toxicologically with the other ingredients in the composition or formulation.

Unless specified otherwise, the term “compounds of the present disclosure” refers to a compound of any structural formula depicted herein (e.g., Compound 1, a compound of Formula I), as well as isomers, such as stereoisomers (including diastereoisomers, enantiomers and racemates), geometrical isomers, conformational isomers (including rotamers and atropisomers), tautomers, isotopically labeled compounds (including deuterium substitutions), and inherently formed moieties (e.g., polymorphs and/or solvates, such as hydrates) thereof. “Enantiomers” are a pair of stereoisomers that are non-superimposable mirror images of each other. A 1:1 mixture of a pair of enantiomers is a “racemic” mixture. A mixture of a pair of enantiomers that is other than 1:1 is an “intermediate” mixture. “Racemate” or “racemic” is used to designate a racemic mixture where appropriate. When a moiety is present that is capable of forming a salt, then salts are included as well, in particular, pharmaceutically acceptable salts. In some embodiments, the recitation of a “compound” is intended to encompass both free compounds and pharmaceutically acceptable salts thereof. As a convention, the phrase “or a pharmaceutically acceptable salt thereof” is explicitly recited when the structural formula of the compound is explicitly recited, but no difference in inclusion or exclusion of pharmaceutically acceptable salts is thereby intended. In a particular embodiment, the term “compound” refers to the compound or a pharmaceutically acceptable salt thereof.

When reference is made to the administration of Compound 1 herein, it will be understood that Compound 1 or a pharmaceutically acceptable salt thereof can be administered unless Compound 1 is defined as its free form. When a dose of Compound 1 is given without mention of any salt forms, it will be understood that Compound 1 can be administered as the free form or the salt, and that the dose can be based on the weight of the free form of Compound 1 or its salt. Likewise, when a dose is expressed for Compound 1 or a salt thereof, it will be understood that the dose can be based on the weight of the free form of Compound 1 or its salt.

Compounds of the present disclosure may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemic mixtures, individual isomers (e.g., diastereomers, enantiomers, geometrical isomers, conformational isomers (including rotamers and atropisomers), tautomers and intermediate mixtures, with all possible isomers and mixtures thereof being included in the present disclosure.

The “enantiomeric excess” or “% enantiomeric excess” of a composition can be calculated using the equation shown below. In the example shown below, a composition contains 90% of one enantiomer, e.g., the S enantiomer, and 10% of the other enantiomer, e.g., the R enantiomer. In this example, %ee = (90-10)/100 = 80%.

Thus, a composition containing 90% of one enantiomer and 10% of the other enantiomer is said to have an enantiomeric excess of 80%. Some compositions described herein contain an enantiomeric excess of at least about 50%, 75%, 90%, 95%, or 99% of the S enantiomer. In other words, the compositions contain an enantiomeric excess of the S enantiomer over the R enantiomer. In other embodiments, some compositions described herein contain an enantiomeric excess of at least about 50%, 75%, 90%, 95%, or 99% of the R enantiomer. In other words, the compositions contain an enantiomeric excess of the R enantiomer over the S enantiomer.

For instance, an isomer/enantiomer can, in some embodiments, be provided substantially free of the corresponding enantiomer, and can also be referred to as “optically enriched,” “enantiomerically enriched,” “enantiomerically pure” and “non-racemic,” as used interchangeably herein. These terms refer to compositions in which the percent by weight of one enantiomer is greater than the amount of that one enantiomer in a control mixture of the racemic composition (e.g., greater than 1:1 by weight). For example, an enantiomerically enriched preparation of the S enantiomer means a preparation of the compound having greater than about 50% by weight of the S enantiomer relative to the R enantiomer, such as at least about 75% by weight, further such as at least about 80% by weight. In some embodiments, the enrichment can be much greater than about 80% by weight, providing a “substantially enantiomerically enriched,” “substantially enantiomerically pure” or a “substantially non-racemic” preparation, which refers to preparations of compositions which have at least about 85% by weight of one enantiomer relative to other enantiomer, such as at least about 90% by weight, and further such as at least 95% by weight. In certain embodiments, the compound provided herein is made up of at least about 90% by weight of one enantiomer. In other embodiments, the compound is made up of at least about 95%, 98%, or 99% by weight of one enantiomer.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, the compound mixture has an (S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

In other embodiments, the compound mixture contains identical chemical entities except for their stereochemical orientations, namely (S)- or (R)-isomers. For example, if a compound disclosed herein has --CH(R)-- unit, and R is not hydrogen, then the --CH(R)-- is in an (S)- or (R)-stereochemical orientation for each of the identical chemical entities. In some embodiments, the mixture of identical chemical entities is a racemic mixture of (S)- and (R)-isomers. In another embodiment, the mixture of the identical chemical entities (except for their stereochemical orientations), contain predominately (S)-isomers or predominately (R)-isomers. For example, the (S)-isomers in the mixture of identical chemical entities are present at about 55%, about 60%, about 65%, about 70%, about 75%, about 80%, about 85%, about 90%, about 95%, about 96%, about 97%, about 98%, about 99%, about 99.5%, or more, relative to the (R)-isomers. In some embodiments, the (S)-isomers in the mixture of identical chemical entities are present at an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

In another embodiment, the (R)-isomers in the mixture of identical chemical entities (except for their stereochemical orientations), are present at about 55%, about 60%, about 65%, about 70%, about 75%, about 80%, about 85%, about 90%, about 95%, about 96%, about 97%, about 98%, about 99%, about 99.5%, or more, relative to the (S)-isomers. In some embodiments, the (R)-isomers in the mixture of identical chemical entities (except for their stereochemical orientations), are present at a (R)-enantiomeric excess greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

Geometric isomers may occur when a compound contains a double bond or some other feature that gives the molecule a certain amount of structural rigidity. If the compound contains a double bond, the double bond may be E- or Z-configuration. If the compound contains a disubstituted cycloalkyl, the cycloalkyl substituent may have a cis- or trans-configuration.

Conformational isomers (or conformers) are isomers that can differ by rotations about one or more bonds. Rotamers are conformers that differ by rotation about only a single bond.

The term “atropisomer,” as used herein, refers to a structural isomer based on axial or planar chirality resulting from restricted rotation in the molecule.

Optically active (R)- and (S)- isomers may be prepared using chiral synthons or chiral reagents, or resolved using conventional techniques (e.g., separated on chiral SFC or HPLC chromatography columns, such as CHIRALPAK® and CHIRALCEL® columns available from DAICEL Corp. or other equivalent columns, using the appropriate solvent or mixture of solvents to achieve suitable separation).

The compounds of the present disclosure can be isolated in optically active or racemic forms. Optically active forms may be prepared by resolution of racemic forms or by synthesis from optically active starting materials. All processes used to prepare compounds of the present disclosure and intermediates made therein are considered to be part of the present disclosure. When enantiomeric or diastereomeric products are prepared, they may be separated by conventional methods, for example, by chromatography or fractional crystallization.

Depending on the process conditions, the end products of the present disclosure are obtained either in free (neutral) or salt form. Both the free form and the salts of these end products are within the scope of the present disclosure. If so desired, one form of a compound may be converted into another form. A free base or acid may be converted into a salt; a salt may be converted into the free compound or another salt; a mixture of isomeric compounds of the present disclosure may be separated into the individual isomers.

Pharmaceutically acceptable salts are preferred. However, other salts may be useful, e.g., in isolation or purification steps which may be employed during preparation, and thus, are contemplated to be within the scope of the present disclosure.

As used herein, “pharmaceutically acceptable salts” refers to salts derived from suitable inorganic and organic acids and bases that are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable acid addition salts include, but are not limited to, acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethanedisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate/hydroxymalonate, mandelate, mesylate, methylsulphate, mucate, naphthoate, napsylate, nicotinate, nitrate, octadecanoate, oleate, oxalate, palmitate, pamoate, phenylacetate, phosphate/hydrogen phosphate/dihydrogen phosphate, polygalacturonate, propionate, salicylates, stearate, succinate, sulfamate, sulfosalicylate, tartrate, tosylate, trifluoroacetate and xinafoate salts. In some embodiments, the pharmaceutically acceptable salt of Compound 1 is a hydrochloride salt.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, or copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Examples of organic amines include, but are not limited to, isopropylamine, benzathine, cholinate, diethanolamine, diethylamine, lysine, meglumine, piperazine and tromethamine.

The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Allen, L.V., Jr., ed., Remington: The Science and Practice of Pharmacy, 22nd Edition, Pharmaceutical Press, London, UK (2012), the relevant disclosure of which is hereby incorporated by reference in its entirety.

Compounds of the present disclosure that contain groups capable of acting as donors and/or acceptors for hydrogen bonds may be capable of forming co-crystals with suitable co-crystal formers. These co-crystals may be prepared from compounds of the present disclosure by known co-crystal forming procedures. Such procedures include grinding, heating, co-subliming, co-melting, or contacting in solution compounds of the present disclosure with the co-crystal former under crystallization conditions and isolating co-crystals thereby formed. Suitable co-crystal formers include those described in WO 2004/078163. Hence, the present disclosure further provides co-crystals comprising a compound of the present disclosure and a co-crystal former.

Any formula given herein is also intended to represent unlabeled forms as well as isotopically labeled forms of the compounds. lsotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorous, fluorine, chlorine and iodine, such as ²H, ³H, ¹¹C, ¹³C, ¹⁴C, ¹⁵N, ¹⁸F, ³¹P, ³²P, ³⁵S, ³⁶C1, ¹²³I, ¹²⁴I and ¹²⁵I, respectively. The present disclosure includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies.

Further, substitution with heavier isotopes, particularly deuterium (i.e., ²H or D) may afford certain therapeutic advantages resulting from greater metabolic stability, for example, increased in vivo half-life or reduced dosage requirements or an improvement in therapeutic index. It is understood that deuterium in this context is regarded as a substituent of a compound of the present disclosure. The concentration of such a heavier isotope, specifically deuterium, may be defined by the isotopic enrichment factor. The term “isotopic enrichment factor,” as used herein, means the ratio between the isotopic abundance and the natural abundance of a specified isotope. If a substituent in a compound of this present disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically labeled compounds of the present disclosure can generally be prepared by conventional techniques known to those skilled in the art or by processes disclosed in the schemes or in the examples and preparations described below (or analogous processes to those described herein below), by substituting an appropriate or readily available isotopically labeled reagent for a non-isotopically labeled reagent otherwise employed. Such compounds have a variety of potential uses, e.g., as standards and reagents in determining the ability of a potential pharmaceutical compound to bind to target proteins or receptors, or for imaging compounds of this disclosure bound to biological receptors in vivo or in vitro.

International patent publications WO2011/069063 (and corresponding U.S. Pat. No. 8,710,245), WO2019/161238 (and corresponding U.S. Pat. No. 10,815,249), and WO2018/151861 (and corresponding U.S. Pat. No. 11,129,807), each of which are incorporated by reference in their entirety, describe compounds of Formula I, and processes of preparing thereof:

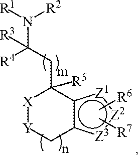

or a pharmaceutically acceptable salt thereof, wherein:
one of X and Y is O, and the other is CH₂; or both X and Y are CH₂;
one of Z¹, Z², and Z³ is S; and (i) two of Z¹, Z², and Z³ are C; or (ii) one of Z¹, Z², and Z³ is C and one of Z¹, Z², and Z³ is N;
R¹ and R² are each independently (i) hydrogen, alkyl, alkoxyl, aminoalkyl, alkenyl, alkynyl, cycloalkyl, cycloalkylalkyl, aryl, or aralkyl, each of which is optionally substituted; or (ii) —(CH₂)ₚ—R⁸, wherein R⁸ is SO₂alkyl or SO₂aryl, each of which is optionally substituted; or (iii) R¹ and R² together with the nitrogen atom to which they are attached form an optionally substituted heterocyclyl or heteroaryl;
R³ and R⁴ are each independently (i) hydrogen, alkyl, alkoxyl, aminoalkyl, alkenyl, alkynyl, cycloalkyl, cycloalkylalkyl, aryl, or aralkyl, each of which is optionally substituted; or (ii) —(CH₂)ₚ—R⁹, wherein R⁹ is CF₃, CN, nitro, amino, hydroxyl, or cycloalkoxyl, each of which is optionally substituted; or (iii) R³ and R⁴ together with the carbon atom to which they are attached form an optionally substituted cycloalkyl or heterocyclyl; or (iv) R³ and R¹ together with the atoms to which they are attached form an optionally substituted heterocyclyl, and R⁴ is (i) or (ii); or (v) R³ and R⁴ are combined together to form a double bond and together with R¹ and/or R² and the atoms to which they are attached form an optionally substituted heteroaryl (e.g., imidazolyl or thiazolyl);
R⁵ is (i) hydrogen, alkyl, alkoxyl, aminoalkyl, alkenyl, alkynyl, cycloalkyl, cycloalkylalkyl, aryl, or aralkyl, each of which is optionally substituted; or (ii) —(CH₂)ₚ—R¹⁰, wherein R¹⁰ is CF₃, CN, nitro, amino, hydroxyl, or cycloalkoxyl, each of which is optionally substituted; or (iii) R⁵ and R¹ together with the atoms to which they are attached form an optionally substituted heterocyclyl;
R⁶ and R⁷ are each independently (i) hydrogen, halo, alkyl, alkoxyl, aminoalkyl, alkenyl, alkynyl, cycloalkyl, cycloalkylalkyl, aryl, or aralkyl, each of which is optionally substituted; or (ii) —(CH₂)ₚ—R¹¹, wherein R¹¹ is CF₃, CN, nitro, amino, hydroxyl, cycloalkoxyl, heteroaryl, or heterocyclyl, each of which is optionally substituted; or (iii) R⁶ and R⁷ together with the atoms to which they are attached form an optionally substituted aryl, heteroaryl, cycloalkyl or heterocyclyl ring; with the proviso that when one of Z¹, Z², and Z³ is N, R⁷ is absent;
m is 0, 1, or 2;
n is 0, 1, or 2; and
each occurrence of p is independently 0, 1, or 2.

International patent publications WO2011/069063 (and corresponding U.S. Pat. No. 8,710,245), WO2019/161238 (and corresponding U.S. Pat. No. 10,815,249), WO2018/151861 (and corresponding U.S. Pat. No. 11,129,807), WO2019/161236, WO2020/118032, and international patent application no. PCT/US2021/026953 describe Compound 1, methods of treating psychiatric disorders using Compound 1, and processes of preparing thereof:

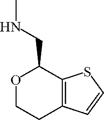

or a pharmaceutically acceptable salt thereof.

Methods of using Compound 1 for the treatment of the psychiatric disorders described herein, particularly schizophrenia, depression, and anxiety, are described in WO2020/118032 (Hopkins SC, Methods of Treating Neurological and Psychiatric Disorders) and Dedic N. et al., SEP-363856, a Novel Psychotropic Agent with a Unique, Non-D2 Receptor Mechanism of Action, J Pharmacol Exp Ther 371:1-14, October 2019.

The chemical name for Compound 1 is (S)-(4,5-dihydro-7H-thieno[2,3-c]pyran-7-yl)-N-methylmethanamine (which may be abbreviated as “(S)-TPMA”), or a pharmaceutically acceptable salt thereof. One having ordinary skill in the art would appreciate the variety of nomenclature for compounds. Accordingly, Compound 1 may also be identified as (S)-1-(4,7-dihydro-5H-thieno[2,3-c]pyran-7-yl)-N-methylmethanamine, (S)-1-(5,7-dihydro-4H-thieno[2,3-c]pyran-7-yl)-N-methylmethanamine, or others, or a pharmaceutically acceptable salt thereof. For example, Compound 1, or a pharmaceutically acceptable salt thereof, is also known as SEP-0363856 or SEP-856 or ulotaront.

Compound 1 can be used in the methods described herein as the free base or in the form of a pharmaceutically acceptable salt. In some embodiments, a hydrochloric acid (HCl) salt of Compound 1 is used in the methods described herein.

Compound 1, or a pharmaceutically acceptable salt thereof, may be in amorphous or crystalline form. In some embodiments, a crystalline form of Compound 1, or a pharmaceutically acceptable salt thereof, is used in the methods described herein. In some embodiments, crystalline Form A of the HCl salt of Compound 1 is used in the methods described herein.

In some embodiments, crystalline Form A of the HCl salt of Compound 1 is characterized by a powder X-ray diffraction pattern comprising peaks, in terms of 2-theta, at 9.6 ± 0.2°., 14.9 ± 0.2°, 20.5 ± 0.2°, and 25.1 ± 0.2°, in some embodiments further comprising peaks at 20.2 ± 0.2° and 20.8 ± 0.2°, and in some embodiments further comprising peaks at 20.2 ± 0.2° and 20.8 ± 0.2° and a peak at two or more of 17.9 ± 0.2°, 24.8 ± 0.2° and 27.1 ± 0.2°.

In some embodiments, Compound 1, or a pharmaceutically acceptable salt thereof, is substantially enantiomerically pure. In some examples, a composition comprising Compound 1, or a pharmaceutically acceptable salt thereof, comprises greater than or equal to about 90%, 95%, 97%, 99%, 99.5%, 99.7% or 99.9% of Compound 1, relative to the total amount of Compound 1 and its (R)-enantiomer in the composition. In some embodiments, a substantially enantiomerically pure crystalline Form A of the HCl salt of Compound 1 is used in the methods described herein.

### Methods

The Diagnostic and Statistical Manual of Mental Disorders, Fifth Ed., (the “DSM-5”), published by the American Psychiatric Association in 2013, and as amended or supplemented, provides a standard diagnostic system upon which persons of skill rely for diagnosis of various diseases and disorders, and is hereby incorporated by reference in its entirety. The DSM-5 attempts to capture the large proportion of patients with subsyndromal mixed symptoms with the inclusion of the mixed specifier. Additionally, the International Statistical Classification of Diseases (ICD 11) coding system is a recognized system to communicate about specific diagnoses (e.g., in the United States for billing purposes), and is hereby incorporated by reference in its entirety. For example, Chapter 5 of the ICD 11 is directed to codes for endocrine, nutritional or metabolic diseases and Chapter 6 of the ICD 11 is directed to codes for mental, behavioral, or neurodevelopmental disorders.

The methods of the disclosure relate to the use of compounds and compositions disclosed herein to treat, or lessen the severity of, metabolic disorders, and to treat psychiatric disorders and diseases such as schizophrenia, depression, and anxiety, in patients with comorbid metabolic disorders. In some embodiments, the metabolic disorder is described in Chapter 5 of the International Statistical Classification of Diseases (ICD 11) coding system. In some embodiments, the metabolic disorder is described in Chapter 5 of the International Statistical Classification of Diseases (ICD 11) coding system as one of an endocrine, nutritional or metabolic disorder.

In an embodiment, the disclosure relates to a method of treating a patient having a metabolic disorder, comprising orally administering to the patient an effective amount of Compound 1, or a pharmaceutically acceptable salt thereof, or pharmaceutical composition disclosed herein.

In an embodiment, the disclosure relates to a method of treating a patient having a metabolic disorder, comprising orally administering to the patient an effective amount of Compound 1, or a pharmaceutically acceptable salt thereof, disclosed herein.

In an embodiment, the disclosure relates to a method of treating a patient having a metabolic disorder, comprising orally administering to the patient Compound 1 or a pharmaceutically acceptable salt thereof. In some embodiments, the method relates to wherein the metabolic disorder is Obesity [including obesity comorbidities including but not limited to metabolic syndrome, dyslipidemia, Type III dyslipidemia, hypertension, insulin resistance, diabetes (including Type 1 and Type 2 diabetes), coronary artery disease, and heart failure]; Overweightness or increased weight; Increased body mass index; Metabolic syndrome; Diabetes [including diabetes-related disorders including Type 1 diabetes (insulin-dependent diabetes mellitus, IDDM) and Type 2 diabetes (non-insulin-dependent diabetes mellitus, NIDDM)]; Diabetic complications (including, but not limited to atherosclerosis, coronary heart disease, stroke, peripheral vascular disease, nephropathy, hypertension, neuropathy and nephropathy); Impaired glucose tolerance; Elevated blood glucose; Insulin resistance; Insulin insensitivity; Hyperglycemia; Fatty liver disease; Non-alcoholic fatty liver disease; Hepatic insulin resistance; Glycosuria; Increased blood triglycerides; Increased appetite; or Dyslipidemia.

The metabolic disorder can be based on various criteria or combinations of criteria. In one embodiment, the subject has waist circumference ≥ 40 inches (101.6 cm) for men or ≥ 35 inches (88.9 cm) for women. In another embodiment, the subject has triglyceride concentration ≥ 150 mg/dL (1.69 mmol/L) or ongoing treatment with triglyceride lowering medication. In another embodiment the subject has (i) HDL-cholesterol concentration < 40 mg/dL (1.03 mmol/L) for men or < 50 mg/dL (1.29 mmol/L) for women or (ii) ongoing treatment with cholesterol lowering medication. In another embodiment, the subject has fasting glucose concentrations ≥ 100 mg/dL (5.6 mmol/L). In one embodiment, the subject has (i) standing or supine blood pressure ≥ 130/85 mmHg or (ii) ongoing treatment with antihypertensive medication.

In one embodiment, the subject has three, four or five of the following criteria (a) - (e): (a) waist circumference ≥ 40 inches (101.6 cm) for men or ≥ 35 inches (88.9 cm) for women; (b) triglyceride concentration ≥ 150 mg/dL (1.69 mmol/L) or ongoing treatment with triglyceride lowering medication; (c) (i) HDL-cholesterol concentration < 40 mg/dL (1.03 mmol/L) for men or < 50 mg/dL (1.29 mmol/L) for women or (ii) ongoing treatment with cholesterol lowering medication; (d) fasting glucose concentrations ≥ 100 mg/dL (5.6 mmol/L); and (e) (i) standing or supine blood pressure ≥ 130/85 mmHg or (ii) ongoing treatment with antihypertensive medication.

In one embodiment, the subject has glycemic derived insulin resistance as evidenced by 5.7%≤HbA1c≤6.4%. In one embodiment, the subject has glycemic derived insulin resistance as evidenced by fasting HOMA-IR ([Insulin uIU/ml x Glucose mg/dl] / 405) ≥ 2.22.

In one embodiment, the subject has three, four or five of the following criteria (a) - (e): (a) waist circumference ≥ 40 inches (101.6 cm) for men or ≥ 35 inches (88.9 cm) for women; (b) triglyceride concentration ≥ 150 mg/dL (1.69 mmol/L) or ongoing treatment with triglyceride lowering medication; (c) (i) HDL-cholesterol concentration < 40 mg/dL (1.03 mmol/L) for men or < 50 mg/dL (1.29 mmol/L) for women or (ii) ongoing treatment with cholesterol lowering medication; (d) fasting glucose concentrations ≥ 100 mg/dL (5.6 mmol/L); and (e) (i) standing or supine blood pressure ≥ 130/85 mmHg or (ii) ongoing treatment with antihypertensive medication; and further wherein the subject has glycemic derived insulin resistance as evidenced by 5.7% ≤ HbA1c ≤ 6.4% or fasting HOMA-IR ([Insulin uIU/ml x Glucose mg/dl] / 405) ≥ 2.22.

In one embodiment, the subject has diabetes. In another embodiment, the subject is on medication for diabetes. In one embodiment, the subject has metabolic syndrome. In one embodiment, the subject is on medication for metabolic syndrome. In one embodiment, the subject has insulin resistance. In one embodiment, the subject is on medication for insulin resistance. In one embodiment the subject has insulin insensitivity. In one embodiment, the subject has insulin sensitivity based on the glucose rate of appearance, glucose rate of disappearance, and/or glycerol rate of appearance. In another embodiment, the subject is on medication for insulin insensitivity. In one embodiment, the subject has impaired glucose tolerance. In another embodiment, the subject has impaired glucose tolerance based on the patient’s insulin, C-peptide, and/or glucose response to feeding. In another embodiment, the subject has impaired glucose tolerance based on a multiplex of metabolic biomarkers selected from the group consisting of glucose, c peptide, insulin, glucagon, leptin, GLP-1, and combinations thereof. In one embodiment, the subject has impaired glucose tolerance based on markers of β cell function and incretins. In one embodiment, the subject is on medication for impaired glucose tolerance. In one embodiment, the subject has elevated blood glucose. In one embodiment the subject is on medication for elevated blood glucose. In some embodiments, the metabolic disorders are associated with a neurological or psychiatric disease or disorder. In other embodiments, the psychiatric disease or disorder is associated with a metabolic disorder. In one embodiment, the neurological or psychiatric disease or disorder is schizophrenia. In one embodiment, the psychiatric disease or disorder is depression. In one embodiment, the neurological or psychiatric disease or disorder is adjunctive major depressive disorder (aMDD). In one embodiment, the psychiatric disease or disorder is anxiety. In one embodiment, the neurological or psychiatric disease or disorder is generalized anxiety disorder (GAD).

It is known that Compound 1 is used in treatment of schizophrenia by once daily dosing, typically at bedtime. Concentrations of Compound 1 following bedtime administration are characterized by peak concentrations during the nighttime, and trough concentrations during the daytime. Peak concentrations with a 50 mg bedtime dose are typically above 100 ng/mL, whereas trough concentrations are typically below 100 ng/mL. Morning time doses of Compound 1 increase the concentrations during the daytime. Morning doses are initiated at 12.5 mg and escalated by 2-fold every 2 days until 50 mg morning doses. Target peak-trough concentrations are 200 to 100 ng/mL at steady state, when administered 50 mg at bedtime and 50 mg in the morning. The morning does is titrated. A titration schedule is to initiate dosing at 12.5 mg and escalating by 2-fold every 2 days until the 50 mg dose is achieved. More gradual or aggressive titration schedules may be used if desired.

It was found that Compound 1 is useful in treating metabolic disorders, and in treating psychiatric disorders and diseases associated with metabolic disorders, as illustrated by several preclinical studies in rats and mice as well as in clinical studies in humans (See Examples).

In an embodiment, the disclosure relates to a method of treating schizophrenia in a human subject in need thereof with comprising:
(a) administering to said subject a therapeutically effective amount of Compound 1 at the subject’s bedtime (“evening dose”); and
(b) administering to said subject a therapeutically effective amount of Compound 1 in the morning (“morning dose”).

In some embodiments, the morning dose is from about 12.5 mg to about 150 mg. In some embodiments, the morning dose is from about 12.5 mg to about 25 mg, or from about 12.5 mg to about 37.5 mg, or from about 12.5 mg to about 50 mg, or from about 12.5 mg to about 75 mg, or from about 12.5 mg to about 100 mg, or from about 12.5 mg to about 125 mg. In some embodiments, the morning dose is about 12.5 mg, or is about 25 mg, or is about 37.5 mg, or is about 50 mg, or is about 75 mg, or is about 100 mg, or is about 125 mg. These ranges can also be used to describe other embodiments disclosed herein.

In some embodiments, the evening dose is from about 12.5 mg to 150 mg. In some embodiments, the evening dose is from about 12.5 mg to about 25 mg, or from about 12.5 mg to about 37.5 mg, or from about 12.5 mg to about 50 mg, or from about 12.5 mg to about 75 mg, or from about 12.5 mg to about 100 mg, or from about 12.5 mg to about 125 mg. In some embodiments, the evening dose is about 12.5 mg, or is about 25 mg, or is about 37.5 mg, or is about 50 mg, or is about 75 mg, or is about 100 mg, or is about 125 mg. These ranges can also be used to describe other embodiments disclosed herein.

In some embodiments, one of the morning dose or the evening dose is about 12.5 mg and the other of the morning dose or the evening dose is about 50 mg. In some embodiments, one of the morning dose or the evening dose is about 25 mg and the other of the morning dose or the evening dose is about 50 mg. In some embodiments, one of the morning dose or the evening dose is about 37.5 mg and the other of the morning dose or the evening dose is about 50 mg. In some embodiments, one of the morning dose or the evening dose is about 50 mg and the other of the morning dose or the evening dose is about 50 mg. In some embodiments, the evening dose is about 50 mg, and the morning dose is about 50 mg, or about 37.5 mg, or about 25 mg, or about 12.5 mg. These ranges can also be used to describe other embodiments disclosed herein.

In some embodiments, the total daily dose does not exceed about 150 mg. In some embodiments, the total daily dose does not exceed about 125 mg. In some embodiments, the total daily dose does not exceed about 100 mg. In some embodiments, the total daily dose does not exceed about 87.5 mg. In some embodiments, the total daily dose does not exceed about 75 mg. In some embodiments, the total daily dose does not exceed about 67.5 mg. In some embodiments, the total daily dose does not exceed about 50 mg. In some embodiments, the total daily dose does not exceed about 37.5 mg. In some embodiments, the total daily dose does not exceed about 25 mg. These ranges can also be used to describe other embodiments disclosed herein.

In one embodiment, the morning dose is administered up to 4 hours prior to or with a meal. In one embodiment, the morning dose is administered up to 4 hours prior to a meal. In one embodiment, the morning dose is administered with a meal. In one embodiment, the evening dose is administered up to 4 hours prior to or with a meal. In one embodiment, the evening dose is administered up to 4 hours prior to a meal. In one embodiment, the evening dose is administered with a meal.

In one embodiment, each of the morning dose and evening dose is administered up to 4 hours prior to or with a meal. In one embodiment, each of the morning dose and evening dose is administered up to 4 hours prior to a meal. In one embodiment, each of the morning dose and evening dose is administered with a meal. In one embodiment, one of the morning dose and evening dose is administered up to 4 hours prior to a meal, and the other of the morning dose and evening dose is administered with a meal.

In one embodiment the dosage is administered once daily. Indeed, it will be understood that any of the methods described herein that employ twice daily dosing can likewise be practiced using once daily dosing, based on the doses described herein.

In one embodiment the dosage is administered in the fed state. In one embodiment, the Cₘₐₓ and AUC_{0-∞} ratios for Compound 1 and the N-desmethyl metabolite of Compound 1 in the fed and fasted states is greater than 90 or greater than 95%.

It will be understood that therapeutic benefit can be observed when practicing the methods of the present invention after as early as 4 weeks, and that therapy can continue for 8 weeks or more, 12 weeks or more, 26 weeks or more, or 52 weeks or more.

In an embodiment, the disclosure relates to a method of treating schizophrenia in a human subject in need thereof comprising:
(a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and
(b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL.

In some embodiments, the average peak plasma concentration is in a range of about 100 ng/mL to about 400 ng/mL. In some embodiments, the average peak plasma concentration is in a range of about 150 ng/mL to about 400 ng/mL. In some embodiments, the average peak plasma concentration is in a range of about 100 ng/mL to about 350 ng/mL, or about 100 ng/mL to about 300 ng/mL, or about 100 ng/mL to about 250 ng/mL, or about 100 ng/mL to about 200 ng/mL, or about 100 ng/mL to about 150 ng/mL. In some embodiments, the average peak plasma concentration is in a range of about 150 ng/mL to about 400 ng/mL, or about 150 ng/mL to about 350 ng/mL, or about 150 ng/mL to about 300 ng/mL, or about 150 ng/mL to about 250 ng/mL, or about 150 ng/mL to about 200 ng/mL. In some embodiments, the average peak plasma concentration is in a range of about 200 ng/mL to about 400 ng/mL, or about 200 ng/mL to about 350 ng/mL, or about 200 ng/mL to about 300 ng/mL, or about 200 ng/mL to about 250 ng/mL. In some embodiments, the average peak plasma concentration is in a range of about 250 ng/mL to about 400 ng/mL, or about 250 ng/mL to about 350 ng/mL, or about 250 ng/mL to about 300 ng/mL. In some embodiments, the average peak plasma concentration is in a range of about 300 ng/mL to about 400 ng/mL, or about 300 ng/mL to about 350 ng/mL. These ranges can also be used to describe other embodiments disclosed herein.

In some embodiments, the average trough plasma concentration is in a range of about 25 ng/mL to about 200 ng/mL. In some embodiments, the average trough plasma concentration is in a range of about 25 ng/mL to about 150 ng/mL, or about 25 ng/mL to about 100 ng/mL, or about 25 ng/mL to about 75 ng/mL, or about 25 ng/mL to about 50 ng/mL. In some embodiments, the average trough plasma concentration is in a range of about 50 ng/mL to about 200 ng/mL, or about 50 ng/mL to about 150 ng/mL, or about 50 ng/mL to about 100 ng/mL, or about 50 ng/mL to about 75 ng/mL. In some embodiments, the average trough plasma concentration is in a range of about 75 ng/mL to about 200 ng/mL, or about 75 ng/mL to about 150 ng/mL, or about 75 ng/mL to about 100 ng/mL. In some embodiments, the average trough plasma concentration is in a range of about 100 ng/mL to about 200 ng/mL, or about 100 ng/mL to about 150 ng/mL. In some embodiments, the average trough plasma concentration is in a range of about 150 ng/mL to about 200 ng/mL. These ranges can also be used to describe other embodiments disclosed herein.

In some embodiments, the maximum peak plasma concentration is about 400 ng/mL, and the maximum trough plasma concentration is about 200 ng/mL. These ranges can also be used to describe other embodiments disclosed herein.

In some embodiments, the average peak plasma concentration is about 150 ng/mL to about 250 ng/mL and average trough concentration is about 25 ng/mL to 125 ng/mL. In some embodiments, the average peak plasma concentration is about 175 ng/mL to about 225 ng/mL and average trough concentration is about 25 ng/mL to 100 ng/mL. In some embodiments, the average peak plasma concentration is about 150 ng/mL to about 225 ng/mL and average trough concentration is about 50 ng/mL to 100 ng/mL. In some embodiments, the average peak plasma concentration is about 225 ng/mL and average trough concentration is about 90 ng/mL.

In an embodiment, the disclosure relates to a method of treating schizophrenia with weight reduction in a human subject in need thereof comprising:
(a) administering to said subject a therapeutically effective amount of Compound 1 at the subject’s bedtime (“evening dose”); and
(b) administering to said subject a therapeutically effective amount of Compound 1 in the morning (“morning dose”),
and the subject’s weight is reduced after about 4-52 weeks of Compound 1 administration.

In some embodiments, the subject’s weight is reduced after about 4-52 weeks of Compound 1 administration. In some embodiments, the subject’s weight is reduced after about 4-26 weeks of Compound 1 administration. In some embodiments, the subject’s weight is reduced after about 4 weeks of Compound 1 administration, or about 6 weeks, or about 8 weeks, or about 10 weeks, or about 12 weeks. In some embodiments, the subject’s weight is reduced after about 14 weeks of Compound 1 administration, or about 16 weeks, or about 18 weeks, or about 20 weeks. In some embodiments, the subject’s weight is reduced after about 22 weeks of Compound 1 administration, or about 24 weeks, or about 26 weeks. In some embodiments, the subject’s weight is reduced after about 20-52 weeks, or about 4-26 weeks, or about 12-26 weeks, or about 36-52 weeks, or about 8-32 weeks, or about 44-52 weeks, or about 4-12 weeks. These ranges can also be used to describe other embodiments disclosed herein.

In an embodiment, the disclosure relates to a method of treating schizophrenia with weight reduction in a human subject in need thereof comprising:
(a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and
(b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL,
and the subject’s weight is reduced after about 4-52 weeks of Compound 1 administration.

In an embodiment, the disclosure relates to a method of treating schizophrenia and improving insulin secretion efficiency in a type 2 diabetic human subject in need thereof comprising:
(a) administering to said subject a therapeutically effective amount of Compound 1 at the subject’s bedtime (“evening dose”); and
(b) administering to said subject a therapeutically effective amount of Compound 1 in the morning (“morning dose”),
and improving the subject’s insulin secretion efficiency.

In an embodiment, the disclosure relates to a method of treating schizophrenia and improving insulin secretion efficiency in a type 2 diabetic human subject in need thereof comprising:
(a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and
(b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL,
and improving the subject’s insulin secretion efficiency.

Thus, the disclosure relates to a method of treating both schizophrenia and diabetes in a patient having both schizophrenia and type 2 diabetes using the methods described herein.

In an embodiment, the disclosure relates to a method of treating adjunctive major depressive disorder (aMDD) in a human subject in need thereof comprising:
(a) administering to said subject a therapeutically effective amount of Compound 1 at the subject’s bedtime (“evening dose”); and
(b) administering to said subject a therapeutically effective amount of Compound 1 in the morning (“morning dose”).

In an embodiment, the disclosure relates to a method of treating adjunctive major depressive disorder (aMDD) in a human subject in need thereof comprising:
(a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and
(b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL.

In an embodiment, the disclosure relates to a method of treating generalized anxiety disorder (GAD) in a human subject in need thereof comprising:
(a) administering to said subject a therapeutically effective amount of Compound 1 at the subject’s bedtime (“evening dose”); and
(b) administering to said subject a therapeutically effective amount of Compound 1 in the morning (“morning dose”).

In an embodiment, the disclosure relates to a method of treating generalized anxiety disorder (GAD) in a human subject in need thereof comprising:
(a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and
(b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL.

In an embodiment, the disclosure relates to a method of reducing weight in a human subject comprising:
(a) administering to said subject a therapeutically effective amount of Compound 1 at the subject’s bedtime (“evening dose”); and
(b) administering to said subject a therapeutically effective amount of Compound 1 in the morning (“morning dose”),
and the subject’s weight is reduced after about 4-52 weeks of Compound 1 administration.

In an embodiment, the disclosure relates to a method of method of reducing weight in a human subject comprising:
(a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and
(b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL,
and the subject’s weight is reduced after about 4-52 weeks of Compound 1 administration.

In an embodiment, the disclosure relates to a method of treating type 2 diabetes in a human subject comprising:
(a) administering to said subject a therapeutically effective amount of Compound 1 at the subject’s bedtime (“evening dose”); and
(b) administering to said subject a therapeutically effective amount of Compound 1 in the morning (“morning dose”),
to improve efficiency of insulin secretion.

In an embodiment, the disclosure relates to a method of treating type 2 diabetes in a human subject comprising:
(a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and
(b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL,
to improve efficiency of insulin secretion.

In another embodiment, there is provided a method of treating schizophrenia, depression, or anxiety in a human subject in need thereof having an excess body mass index (BMI) defined by a BMI ≥ 25 comprising administering to the subject a therapeutically effective amount of Compound 1.

In one embodiment, there is provided a method of treating schizophrenia, depression, or anxiety in a human subject in need thereof having diabetes or metabolic syndrome comprising administering to the subject a therapeutically effective amount of Compound 1.

Another embodiment provides a method of treating schizophrenia, depression, or anxiety in a human subject in need thereof having a metabolic disorder selected from obesity; overweightness or increased weight; increased body mass index; metabolic syndrome; diabetes or diabetes-related disorders (type 1 diabetes (insulin-dependent diabetes mellitus, IDDM) and Type 2 diabetes (non-insulin-dependent diabetes mellitus, NIDDM)); diabetic complications; impaired glucose tolerance; elevated blood glucose; insulin resistance; insulin insensitivity; hyperglycemia; fatty liver disease; non-alcoholic fatty liver disease; hepatic insulin resistance; glycosuria; increased blood triglycerides; increased appetite; or dyslipidemia, comprising administering to the subject a therapeutically effective amount of Compound 1.

In another embodiment, there is provided a method of treating schizophrenia, depression, or anxiety in a human subject in need thereof having a metabolic disorder selected from insulin resistance, insulin insensitivity, impaired glucose tolerance, and elevated blood glucose comprising administering to the subject a therapeutically effective amount of Compound 1.

Another embodiment provides a method of treating schizophrenia, depression, or anxiety in a human subject in need thereof, wherein the human subject is further in need of delayed gastric emptying, comprising administering to the subject a therapeutically effective amount of Compound 1.

Another embodiment provides a method of treating schizophrenia, depression, or anxiety in a human subject in need thereof, comprising administering to the subject a therapeutically effective amount of Compound 1 in the fed state.

In one embodiment, the subject’s glycemic control is improved as compared to when Compound 1 is not administered. In one embodiment, the subject’s glucose tolerance is improved as compared to when Compound 1 is not administered. In one embodiment, the subject’s satiety is increased as compared to when Compound 1 is not administered. In one embodiment, the subject’s appetite is reduced as compared to when Compound 1 is not administered. In one embodiment, the subject’s glucose excursion is reduced as compared to when Compound 1 is not administered. In one embodiment, the subject’s insulin uptake is improved as compared to when Compound 1 is not administered. In one embodiment, the subject’s body fat is reduced as compared to when Compound 1 is not administered. In one embodiment, the subject’s abdominal body fat is reduced as compared to when Compound 1 is not administered.

In one embodiment, this disclosure provides methods of treating schizophrenia with Compound 1 as described herein, in a human subject in need thereof.

In one embodiment, this disclosure provides methods of treating adjunctive major depressive disorder (aMDD) with Compound 1 as described herein, in a human subject in need thereof.

In one embodiment, this disclosure provides methods of treating generalized anxiety disorder (GAD) with Compound 1 as described herein, in a human subject in need thereof.

Non-limiting examples of classes of metabolic disorders as used herein include, in addition to those previously mentioned:

### Endocrine Diseases Identified in ICD 11

Disorders of the thyroid gland or thyroid hormones system; Diabetes mellitus; Other disorders of glucose regulation or pancreatic internal secretion; Disorders of the parathyroids or parathyroid hormone system; Disorders of the pituitary hormone system; Disorders of the adrenal glands or adrenal hormone system; Disorders of the gonadal hormone system; Certain disorders of puberty; Polyglandular dysfunction; Endocrine disorders, not elsewhere classified; Neoplasms of the endocrine system; Endocrine tumors.

### Nutritional Disorders Identified in ICD 11

Undernutrition; Overweight, obesity or specific nutrient excesses; Nutritional or toxic disorders of the nervous system; Other specified nutritional disorders; Nutritional disorders, unspecified.

### Metabolic Diseases Identified in ICD 11

Inborn errors of metabolism; Disorders of metabolite absorption or transport; Disorders of fluid, electrolyte or acid-base balance; Disorders of lipoprotein metabolism or certain specified lipidaemias; Metabolic or transporter liver disease; Other metabolic disorders; Cystic fibrosis; Metabolic disorders following abortion, ectopic or molar pregnancy; Metabolic disorders, unspecified.

In some embodiments, provided herein are methods of treating metabolic disorders wherein the metabolic disorder is Obesity [including obesity comorbidities including but not limited to metabolic syndrome, dyslipidemia, Type III dyslipidemia, hypertension, insulin resistance, diabetes (including Type 1 and Type 2 diabetes), coronary artery disease, and heart failure]; Overweightness or increased weight; Increased body mass index; Metabolic syndrome; Diabetes [including diabetes-related disorders including Type 1 diabetes (insulin-dependent diabetes mellitus, IDDM) and Type 2 diabetes (non-insulin-dependent diabetes mellitus, NIDDM)]; Diabetic complications (including, but not limited to atherosclerosis, coronary heart disease, stroke, peripheral vascular disease, nephropathy, hypertension, neuropathy and nephropathy); Impaired glucose tolerance; Elevated blood glucose; Insulin resistance; Insulin insensitivity; Hyperglycemia; Fatty liver disease; Non-alcoholic fatty liver disease; Hepatic insulin resistance; Glycosuria; Increased blood triglycerides; Increased appetite; or Dyslipidemia. In some embodiments, provided herein are methods of treating metabolic disorders wherein the metabolic disorder is metabolic syndrome.

Weight gain (increased weight) is the best recognized metabolic side effect of atypical antipsychotics, although the magnitude varies depending on the agent. Excessive weight gain is prominently observed following treatment with olanzapine and clozapine and has been associated with a number of other atypical and typical antipsychotics. In addition, weight gain increases the risk for hyperglycemia and diabetes, which in turn leads to increased cardiovascular disease risk and mortality.

Gastric emptying and GI motility have significant effects on appetite and energy consumption. Inhibition and/or delay of gastric emptying can lead to increased satiety and thereby decrease food intake (Cifuentes et al., 2021). Obesity is associated with multiple metabolic abnormalities including rapid gastric emptying and large fasting gastric volume, which many result in increased food intake and appetite. GLP-1 receptor agonists (e.g., semaglutide and liraglutide) used for the treatment of type 2 diabetes and chronic weight management have been shown to delay gastric emptying in preclinical species and humans (J van Can et al., 2014).

Delaying gastric emptying is one of the mechanisms responsible for the improved glucose tolerance with Compound 1. Additionally, Compound 1 also modulates homeostatic and hedonic neurocircuits governing energy balance and feeding.

In one embodiment, the subject is in need of delayed gastric emptying based on the subject’s fullness, hunger and/or satiety, following a meal. In one embodiment, the subject is in need of delayed gastric emptying based on an increase in BMI subsequent to schizophrenia diagnosis or commencement of antipsychotic therapy. In one embodiment, the subject is in need of delayed gastric emptying based on a ¹³C Spirulina Breath Test (GEBT).

In some embodiments, compounds disclosed herein can be used to treat metabolic disorders or symptoms thereof, such as increased percentage body fat; decreased percentage of lean mass in an individual; high blood pressure (hypertension), cardiovascular disease, hyperglycemia, hyperuricemia, and polycystic ovary syndrome.

Metabolic syndrome (also known as metabolic syndrome X or syndrome X) is a combination of medical disorders that increase the risk of cardiovascular disease. As described in Mottillo et al. JOURNAL OF THE AMERICAN COLLEGE OF CARDIOLOGY 54(14), 1113-32 (2010) and Saof-Ali et al. DIABETOLOGY & METABOLIC SYNDROME 12(67), (2020), a diagnosis of metabolic syndrome requires at least three of the following criteria (U.S. National Cholesterol Education Program (NCEP), 2001 and revised NCEP, 2004):
1) central obesity (waist circumference: men >102 cm (~40 inches); women >88 cm (~35 inches));
2) elevated triglycerides (≥150 mg/dl);
3) diminished high-density lipoprotein (HDL) cholesterol (men <40 mg/dl; women <50 mg/dl);
4) systemic hypertension (raised blood pressure) (systolic ≥130/ diastolic ≥85 mm Hg);
5) elevated fasting glucose (≥110 mg/dl) [rNCEP elevated fasting glucose (≥100 mg/dl)].

However, additional criteria may support a diagnosis for metabolic syndrome or metabolic disorders, such as Body Mass Index (e.g., BMI > 30 kg/m²). Metabolic syndrome may also be related to elevated total cholesterol. Elevated LDL cholesterol is marked by levels above about 100, about 130, about 160 or about 200 mg/dL. Metabolic syndrome may also be related to elevated total cholesterol. Impaired glucose intolerance is defined as a two-hour glucose level (glycemia) of about 140 to about 199 mg/dL (7.8 to 11.0 mmol) on the 75-g oral glucose tolerance test (according to WHO and ADA). Glycemia of about 200 mg/dl or greater is considered diabetes mellitus. Hyperglycemia, or high blood sugar, can be defined as a blood glucose level higher than about 7, about 10, about 15, or about 20 mmol/L. Hypoglycemia, or low blood sugar, can be defined as preprandial blood glucose below about 4 or about 6 mmol/L (72 to 108 mg/dl) or 2-hour postprandial blood glucose below about 5 or about 8 mmol/L (90 to 144 mg/dl). Insulin resistance is defined as a state in which a normal amount of insulin produces a subnormal biologic response. Insulin resistance can be measured by the hyperinsulinemic euglycemic clamp technique, Homeostatic Model Assessment (HOMA), or Quantitative insulin sensitivity check index (QUICKI). Hyperuricemia is an abnormally high level of uric acid in the blood, e.g., above 360 µmol/L (6 mg/dL) for women and 400 µmol/L (6.8 mg/dL) for men. Polycystic ovarian syndrome (PCOS) is associated with oligoovulation, anovulation, excess androgen, and/or polycystic ovaries. Metabolic syndrome may also be associated with acanthosis nigricans. Metabolic syndrome may also be associated with a pro-inflammatory state (e.g., elevated C-reactive protein levels in the blood, e.g., above 10 mg/L) and microalbuminuria (urinary albumin excretion ratio≥20 mg/min or albumin:creatinine ratio≥30 mg/g).

In some embodiments, compounds described herein can be used to treat fatty liver disease or a condition related thereto. The fatty liver disease can be a method of treating nonalcoholic steatohepatitis (NASH), nonalcoholic fatty liver disease (NAFLD), simple fatty liver (steatosis), cirrhosis, hepatitis, liver fibrosis, or steatonecrosis. Fatty liver disease can be assessed by diagnostic methods known in the art including liver enzyme tests (ALT, AST), liver ultrasound, FibroTest®, SteatoTest®, coagulation studies including international normalized ratio (INR), as well as blood tests including M30-Apoptosense ELISA, erythrocyte sedimentation rate, glucose, albumin, and renal function. Fatty liver disease may also be associated with a pro-inflammatory state (e.g., elevated C-reactive protein levels in the blood, e.g., above 10 mg/L) as well as hepatocellular carcinoma. Fatty liver disease may also be associated with abetalipoproteinemia, glycogen storage diseases, Weber-Christian disease, Wolman disease, acute fatty liver of pregnancy, lipodystrophy, inflammatory bowel disease, HIV, and hepatitis C (especially genotype 3), and alpha 1-antitrypsin deficiency.

In some embodiments, compounds described herein can be used to reduce percentage body fat, increase percentage lean mass, or to treat obesity (as well as associated conditions). In some embodiments, compounds described herein can be used to treat class I obesity, class II obesity, class III obesity, elevated body weight, elevated body mass index (BMI), elevated body volume index (BVI), elevated body fat percentage, elevated fat to muscle ratio, elevated waist circumference, or elevated waist-hip ratio. Class I obesity is characterized by a BMI of about 30 to about 35, class II obesity (severe obesity) is characterized by a BMI of about 35 to about 40, and class III obesity (morbid obesity) is characterized by a BMI of 40 or greater. A BMI of greater than about 45 or 50 is considered super obese. Elevated body weight can be assessed in consideration of age, gender, height, frame, and/or ethnicity. Elevated waist-hip ratio is defined as greater than about 0.9 for men and greater than about 0.7 for women.

In one embodiment, the subject has an excess BMI defined by a BMI ≥ 25, 27.5, or 30. In another embodiment, the subject has an excess BMI defined by a BMI ≥ 25, 27.5, or 30 and a history of BMI < 25 kg/m² prior to schizophrenia, depression, or anxiety diagnosis. In another embodiment, the subject has an excess BMI defined by a BMI ≥ 30 and a history of BMI < 25 kg/m² prior to schizophrenia, depression, or anxiety diagnosis, and the subject is on a typical or atypical antipsychotic. In another embodiment, the subject has a BMI ≥ 25, 27.5, or 30 induced by a typical or atypical antipsychotic.

Metabolic disorders are inter-related and can result in disorders across various systems. Addressing the core metabolic disorder can reduce the severity of related conditions in a patient, including, e.g.: cardiovascular disorders including, e.g., ischemic heart disease, angina and myocardial infarction, congestive heart failure, high blood pressure, abnormal cholesterol levels, deep vein thrombosis, and pulmonary embolism; neurological disorders including, e.g., stroke, meralgia paresthetica, migraines, idiopathic, and intracranial hypertension, depression (especially in women) and social stigmatism; rheumatological and orthopedic disorders including, e.g., gout, poor mobility, osteoarthritis, and lower back pain; dermatological disorders including, e.g., stretch marks, acanthosis nigricans, lymphedema, and cellulitis; gastrointestinal disorders including, e.g., gastroesophageal reflux disease (GERD) and cholelithiasis (gallstones); respiratory disorders including, e.g., obstructive sleep apnea, obesity hypoventilation syndrome, asthma, and increased complications during general anesthesia; urology and nephrology disorders including, e.g., erectile dysfunction, urinary incontinence, chronic renal failure, and hypogonadism.

In some embodiments, the metabolic disorder is associated with a neurological or psychiatric disease or disorder. In some embodiments, the neurological or psychiatric disease or disorder is described in the DSM-5, as amended or supplemented, or the International Statistical Classification of Diseases (ICD 11) coding system. Antipsychotics are used to treat psychosis associated with several diseases, including the neurological or psychiatric diseases or disorders, and Compound 1 can also be used for similar purpose.

Non-limiting examples of classes of neurological or psychiatric diseases or disorders include Movement Disorders, Cognitive Disorders, Pain, Neurodevelopmental Disorders; Schizophrenia Spectrum and Other Psychotic Disorders; Bipolar and Related Disorders; Depressive Disorders; Anxiety Disorders; Obsessive-Compulsive and Related Disorders; Trauma- and Stressor-Related Disorders; Dissociative Disorders; Somatic Symptom and Related Disorders; Feeding and Eating Disorders; Elimination Disorders; Sleep-Wake Disorders; Sexual Dysfunctions; Gender Dysphoria; Disruptive, Impulse-Control, and Conduct Disorders; Substance-Related and Addictive Disorders; Neurocognitive Disorders; Personality Disorders; Paraphilic Disorders; Other Mental Disorders; and Medication-Induced Movement Disorders and Other Adverse Effects of Medication.

Non-limiting examples of classes of neurological or psychiatric diseases or disorders include:

### Movement Disorders

Tremor; Dyskinesia; Dystonia; Tics; Dysphonia; Ataxia (e.g., spinocerebellar ataxia); Myoclonus; Essential Tremor; Epilepsy; Tardive Dyskinesia; Restless Leg Syndrome; Tourette Syndrome; Multiple System Atrophy (MSA); Multiple Sclerosis; Huntington’s Disease; Parkinson’s Disease; Parkinsonism; Atypical Parkinsonisms (including, for example, Parkinson’s Disease Tremor); Wilson’s Disease; Stroke. Examples of akinesias and akinetic-rigid syndromes include Parkinson’s disease, drug-induced Parkinsonism, postencephalitic Parkinsonism, secondary Parkinsonism, Parkinson plus syndromes, atypical Parkinsonism, idiopathic Parkinsonism, progressive supranuclear palsy, multiple system atrophy, corticobasal degeneration, Parkinsonism-ALS dementia complex and basal ganglia calcification, medication-induced Parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette’s syndrome, epilepsy, muscular spasms and disorders associated with muscular spasticity or weakness including tremors. Examples of dyskinesias include drug (e.g., L-DOPA) induced dyskinesia tremor (such as rest tremor, postural tremor, intention tremor), chorea (such as Sydenham’s chorea, Huntington’s disease, benign hereditary chorea, neuroacanthocytosis, symptomatic chorea, drug-induced chorea and hemiballism), myoclonus (including generalized myoclonus and focal myoclonus), tics (including simple tics, complex tics and symptomatic tics). Examples of dystonias include generalized dystonia, idiopathic dystonia, drug-induced dystonia, symptomatic dystonia, paroxysmal dystonia, oromandibular dystonia, spasmodic dysphonia, spasmodic torticollis, axial dystonia, dystonic writer’s cramp and hemiplegic dystonia. Other examples of movement diseases or disorders include stereotypic movement disorder, persistent (chronic) motor disorder, medication-Induced movement disorder, psychogenic movement disorders, substance/medication-Induced movement disorder, extrapyramidal movement disorders, hyperkinetic movement disorders, hypokinetic movement disorders, alternating hemiplegia, Angelman syndrome, Hallervorden-Spatz Disease, ataxia, dentate cerebellar ataxia, ataxia telangiectasia (Louis-Bar syndrome), Friedreich’s Ataxia, hereditary spinal ataxia, hereditary spinal sclerosis, Machado-Joseph Disease, spinocerebellar ataxia, progressive myoclonic ataxia, athetosis, ballismus, blepharospasm (eye twitching), cerebral palsy, tardive dystonia, tardive dyskinesia, idiopathic torsion dystonia, torsion dystonia, focal dystonia, idiopathic familial dystonia, Idiopathic nonfamilial dystonia, cervical dystonia (spasmodic torticollis), primary dystonia, orofacial dystonia, developmental coordination disorder, bulbospinal muscular atrophy (Kennedy’s Disease), Shy-Drager Syndrome, and Stiff-Person (Stiff-Man) Syndrome. In some embodiments, the present disclosure provides a method of treating one or more symptoms of epilepsy and/or seizures, including abdominal epilepsy, absence seizure, acquired epilepsy, acquired epileptiform aphasia, Aicardi syndrome, Alpers’ disease, Alpers-Huttenlocher syndrome, Angelman syndrome, benign focal epilepsy, benign focal epilepsy of childhood, benign intracranial hypertension, benign rolandic epilepsy (BRE), CDKL5 disorder, childhood absence epilepsy, dentate cerebellar ataxia, Doose syndrome, Dravet syndrome, dyscognitive focal seizure, epilepsy with grand mal seizures, epilepsy with myoclonic-absences, epileptic hemiplegia, febrile seizures, focal seizure, frontal lobe epilepsy, generalized tonic-clonic seizures, genetic epilepsy, Glut1 deficiency syndrome, hypothalamic hamartoma, idiopathic epilepsy, idiopathic generalized epilepsy, idiopathic localization-related epilepsies, idiopathic partial epilepsy, idiopathic seizure, juvenile absence epilepsy, juvenile myoclonic epilepsy, Lafora disease, Lafora progressive myoclonus epilepsy, Landau-Kleffner syndrome, Lassueur-Graham-Little syndrome, Lennox syndrome, Lennox-Gastaut syndrome, medically refractory epilepsy, mesial-temporal lobe sclerosis, myoclonic seizure, neonatal epilepsy, occipital lobe epilepsy, Ohtahara syndrome, Panayiotopoulos syndrome, parietal lobe epilepsy, PCDH19 epilepsy, photosensitive epilepsy, progressive myoclonic epilepsies, Rasmussen’s encephalitis, Rasmussen’s syndrome, refractory epilepsy, seizure disorder, status epilepticus, Sturge-Weber syndrome, symptomatic generalized epilepsy, symptomatic partial epilepsy, TBCK-related ID syndrome, temporal lobe epilepsy, temporal lobe seizures, tonic-clonic seizure, West syndrome, tremor, cerebellar tremor, cerebellar outflow tremor, intention tremor, essential tremor, benign essential tremor, Parkinsonian tremor, and medication-induced postural tremor.

### Cognitive Disorders

Alzheimer’s disease; Cognitive Impairments; Dementia (including, e.g., Semantic Dementia; Frontotemporal Dementia; Dementia with Depressive Features; Persisting, Subcortical Dementia; Dementia with Lewy Bodies; Parkinsonism-ALS Dementia Complex; Dementia Associated with another disease or disorder, including Alzheimer’s Disease; Ischemia; Multi-Infarct Dementia; Trauma; Vascular Problems; Stroke; HIV Disease; Parkinson’s Disease; Huntington’s Disease; Down Syndrome; Pick’s Disease; Creutzfeldt-Jacob Disease; Perinatal Hypoxia, or Substance abuse), Delirium; Amnestic Disorders; or Age Related Cognitive Decline. Cognitive Disorders includes a decline in cognitive functions or cognitive domains, e.g., working memory, attention and vigilance, verbal learning and memory, visual learning and memory, reasoning and problem solving (e.g., executive function, speed of processing and/or social cognition). In particular, cognitive impairment may indicate deficits in attention, disorganized thinking, slow thinking, difficulty in understanding, poor concentration, impairment of problem solving, poor memory, difficulties in expressing thoughts, and/or difficulties in integrating thoughts, feelings and behavior, or difficulties in extinction of irrelevant thoughts. Cognitive Disorders can manifest as a deficit in cognition (cognitive domains as defined by the DSM-5 are: complex attention, executive function, learning and memory, language, perceptual-motor, social cognition); and is sometimes associated with a deficit in dopamine signaling; and is sometimes associated with basal ganglia dysfunction; and is sometimes associated with dysregulated locomotor activity; and is sometimes associated with impairment of prefrontal cortex functioning.

### Pain

Fibromyalgia; Neuropathic Pain (including, e.g., post herpetic (or post-shingles) neuralgia, reflex sympathetic dystrophy/causalgia or nerve trauma, phantom limb pain, carpal tunnel syndrome, and peripheral neuropathy (such as diabetic neuropathy or neuropathy arising from chronic alcohol use)); Sensitization Accompanying Neuropathic Pain; Inflammatory Pain; Acute Pain; Nociceptive Pain; Arthritis Pain; Rheumatoid Arthritis; Osteoarthritis; Joint Pain; Musculoskeletal Pain; Back Pain; Dorsalgia; Bulging Disc; Hip Pain; Visceral Pain; Headache; Tension Headache; Acute Tension Headache; Chronic Tension Headache; Chronic Cluster Headache; Common Migraine; Classic Migraine; Cluster Headache; Mixed Headache; PostTraumatic Headache; Eye Strain Headache; Short-Lasting Unilateral Neuralgiform (SUNCT) Headache; SUNCT Syndrome; Herpes Zoster; Acute Herpes Zoster; Shingles; Postherpetic Neuralgia (Shingles); Causalgia; Central Pain; Central Pain Syndrome; Chronic Back Pain; Neuralgia; Neuropathic Pain Syndrome; Neuropathy; Diabetic Neuropathy; Diabetes-Related Neuropathy; Diabetes-Related Nerve Pain; Fibrositis; Peripheral Neuropathy Caused by Chemotherapy; Peripheral Nerve Disease; Peripheral Neuropathy; Nerve Pain; Nerve Trauma; Sensitization Accompanying Neuropathic Pain; Complex Regional Pain Syndrome; Compression Neuropathy; Craniofacial Pain; Chronic Joint Pain; Chronic Knee Pain; Chronic Pain Syndrome; Cancer Pain; Trigeminal Neuralgia; Tic Doloreaux; Reflex Sympathetic Causalgia; Painful Peripheral Neuropathy; Spinal Nerve Injury; Arachnoiditis; Spinal Pain; Bernhardt-Roth Syndrome (Meralgia Parasthetica); Carpal Tunnel Syndrome; Cerebrospinal Fluid Syndrome; Charcot-Marie-Tooth Disease; Hereditary Motor and Sensory Neuropathy; Peroneal Muscular Atrophy; Cluster-Tic Syndrome; Coccygeal Pain Syndromes; Compartment Syndrome; Degenerative Disc Disease; Failed Back Surgery Syndrome; Genito-Pelvic Pain/Penetration Disorder; Gout; Inflammatory Pain; Lumbar Radiculopathy; Neuroma (Painful Scar); Pain Associated with Multiple Sclerosis; Pelvic Floor Disorders; Phantom Limb Pain; Piriformis Syndrome; Psychogenic Pain; Radicular Pain Syndrome; Raeder’s Syndrome; Referred Pain; Reflex Sympathetic Dystrophy Syndrome; Sciatica; Sciatica Pain: Scoliosis; Slipped Disc; Somatic Pain; Spinal Stenosis; Stiff-Person Syndrome/Stiff-Man Syndrome; Stump Pain; Sympathetically Maintained Pain; Tolosa-Hunt Syndrome; Whiplash; Pain Associated with Lyme Disease.

### Neurodevelopmental Disorders

Intellectual Disability (Intellectual Developmental Disorder); Global Developmental Delay; Unspecified Intellectual Disability (Intellectual Developmental Disorder); Language Disorder; Speech Sound Disorder; Childhood-Onset Fluency Disorder (Stuttering); Social (Pragmatic) Communication Disorder; Unspecified Communication Disorder; Autism Spectrum Disorder (including, e.g., Asperger’s syndrome; Pervasive Developmental Disorder; Rett Syndrome; and Fragile X Syndrome); Attention-Deficit/Hyperactivity Disorder; Other Specified Attention-Deficit/Hyperactivity Disorder; Unspecified Attention-Deficit/ Hyperactivity Disorder; Specific Learning Disorder; Childhood Learning Disorder; Developmental Coordination Disorder; Stereotypic Movement Disorder; Tic Disorders; Other Specified Tic Disorder; Unspecified Tic Disorder; Other Specified Neurodevelopmental Disorder; Unspecified Neurodevelopmental Disorder.

### Schizophrenia Spectrum and Other Psychotic Disorders

Schizotypal (Personality) Disorder; Delusional Disorder; Brief Psychotic Disorder; Shared Psychotic Disorder Schizophreniform Disorder; Schizophrenia (paranoid, disorganized, catatonic, or undifferentiated); Schizoaffective Disorder; Substance/Medication-Induced Psychotic Disorder; Psychotic Disorder Due to Another Medical Condition; Catatonia Associated With Another Mental Disorder (Catatonia Specifier); Catatonic Disorder Due to Another Medical Condition; Unspecified Catatonia; Other Specified Schizophrenia Spectrum and Other Psychotic Disorder;, Unspecified Schizophrenia Spectrum and Other Psychotic Disorder. Schizophrenia is a disorder of unknown origin, which usually appears for the first time in early adulthood and is marked by characteristics such as psychotic symptoms, phasic progression and development, and/or deterioration in social behavior and professional capability. Characteristic psychotic symptoms are disorders of thought content (e.g., multiple, fragmentary, incoherent, implausible or simply delusional contents, or ideas of persecution) and of mentality (e.g., loss of association, flight of imagination, incoherence up to incomprehensibility), as well as disorders of perceptibility (e.g., hallucinations), emotions (e.g., superficial or inadequate emotions), self-perceptions, intentions, impulses, and/or inter-human relationships, and psychomotoric disorders (e.g., catatonia). Other symptoms are also associated with this disorder. Schizophrenia is classified into subgroups: the paranoid type, characterized by delusions and hallucinations and absence of thought disorder, disorganized behavior, and affective flattening; the disorganized type, also named “hebephrenic schizophrenia,” in which thought disorder and flat affect are present together; the catatonic type, in which prominent psychomotor disturbances are evident, and symptoms may include catatonic stupor and waxy flexibility; and the undifferentiated type, in which psychotic symptoms are present but the criteria for paranoid, disorganized, or catatonic types have not been met. The symptoms of schizophrenia normally manifest themselves in three broad categories: positive, negative, and cognitive symptoms. Positive symptoms are those which represent an “excess” of normal experiences, such as hallucinations and delusions. Negative symptoms are those where the subject suffers from a lack of normal experiences, such as anhedonia and lack of social interaction. The cognitive symptoms relate to cognitive impairment in schizophrenics, such as lack of sustained attention and deficits in decision making.

In one embodiment, the subject is in need of treatment for schizophrenia. In another embodiment, the subject is in need of treatment for schizophrenia, and the subject has a CGI-S score ≤ 4 (normal to moderately ill), a SAS score < 2, an AIMS score < 3, and a BARS score < 3. In one embodiment, the subject is in need of treatment for schizophrenia, and the subject has a positive and negative symptom scale (PANSS) total score ≤ 80.

### Bipolar and Related Disorders

Bipolar I Disorder; Bipolar II Disorder; Cyclothymic Disorder; Substance/Medication-Induced Bipolar and Related Disorder; Bipolar and Related Disorder Due to Another Medical Condition; Other Specified Bipolar and Related Disorder; Unspecified Bipolar and Related Disorder; Specifiers for Bipolar and Related Disorders. Bipolar disorders (including both bipolar I and bipolar II) are serious psychiatric disorders that have a prevalence of approximately 2% of the population, and affect both genders alike. They are relapsing-remitting conditions characterized by cycling between elevated (i.e., manic) and depressed moods, which distinguishes them from other disorders such as major depressive disorder and schizophrenia. Bipolar I is defined by the occurrence of a full manic episode, although most individuals experience significant depression. Symptoms of mania include elevated or irritable mood, hyperactivity, grandiosity, decreased need for sleep, racing thoughts, and in some cases, psychosis. The depressive episodes are characterized by anhedonia, sad mood, hopelessness, poor self-esteem, diminished concentration, and lethargy. Bipolar II is defined as the occurrence of a major depressive episode and hypomanic (less severe mania) episode although subjects spend considerably more time in the depressive state. Other related conditions include cyclothymic disorder.

### Depressive Disorders

Depression; Disruptive Mood Dysregulation Disorder; Major Depressive Disorder (MDD) (Unipolar Depression); Adjunctive Major Depressive Disorder (aMDD), Persistent Depressive Disorder (Dysthymia); Premenstrual Dysphoric Disorder; Substance/Medication-Induced Depressive Disorder; Treatment-Resistant Depression; Depressive Disorder Due to Another Medical Condition; Other Specified Depressive Disorder; Unspecified Depressive Disorder.

### Anxiety Disorders

Anxiety; Separation Anxiety Disorder; Selective Mutism; Specific Phobia; Social Anxiety Disorder (Social Phobia); Panic Disorder; Panic Attack Specifier; Agoraphobia; Generalized (or General) Anxiety Disorder; Substance/Medication-Induced Anxiety Disorder; Anxiety Disorder Due to Another Medical Condition; Other Specified Anxiety Disorder; Unspecified Anxiety Disorder. Anxiety disorders are characterized by fear, worry, and uneasiness, usually generalized and unfocused as an overreaction to a situation. Anxiety disorders differ in the situations or types of objects that induce fear, anxiety, or avoidance behavior, and the associated cognitive ideation. Anxiety differs from fear in that anxiety is an emotional response to a perceived future threat while fear is associated with a perceived or real immediate threat. They also differ in the content of the associated thoughts or beliefs. Examples of anxiety disorders include separation anxiety disorder, selective mutism, specific phobia, social anxiety disorder (social phobia), panic disorder, panic attack specifier, agoraphobia, generalized anxiety disorder, substance/medication-induced anxiety disorder, anxiety disorder due to another medical condition, illness anxiety disorder, social (pragmatic) communication disorder, other specified anxiety disorder, and unspecified anxiety disorder; stressor-related disorders, including reactive attachment disorder, disinhibited social engagement disorder, posttraumatic stress disorder (PTSD), acute stress disorder, and adjustment disorders.

### Obsessive-Compulsive and Related Disorders

Obsessive-Compulsive Disorder; Body Dysmorphic Disorder; Hoarding Disorder; Trichotillomania (Hair-Pulling Disorder); Excoriation (Skin-Picking) Disorder; Substance/Medication-Induced Obsessive-Compulsive and Related Disorder; Obsessive-Compulsive and Related Disorder Due to Another Medical Condition; Other Specified Obsessive-Compulsive and Related Disorder; Unspecified Obsessive-Compulsive and Related Disorder.

### Trauma- and Stressor-Related Disorders

Reactive Attachment Disorder; Disinhibited Social Engagement Disorder; Posttraumatic Stress Disorder; Acute Stress Disorder; Adjustment Disorders; Other Specified Trauma- and Stressor-Related Disorder; Unspecified Trauma- and Stressor-Related Disorder.

### Dissociative Disorders

Dissociative Identity Disorder; Dissociative Amnesia; Depersonalization/Derealization Disorder; Other Specified Dissociative Disorder; Unspecified Dissociative Disorder.

### Somatic Symptom and Related Disorders

Somatic Symptom Disorder; Illness Anxiety Disorder; Conversion Disorder (Functional Neurological Symptom Disorder); Psychological Factors Affecting Other Medical Conditions; Factitious Disorder; Other Specified Somatic Symptom and Related Disorder; Unspecified Somatic Symptom and Related Disorder.

### Feeding and Eating Disorders

Pica; Rumination Disorder; Avoidant/Restrictive Food Intake Disorder; Anorexia Nervosa; Bulimia Nervosa; Binge-Eating Disorder; Other Specified Feeding or Eating Disorder; Unspecified Feeding or Eating Disorder.

### Elimination Disorders

Enuresis; Encopresis; Other Specified Elimination Disorder; Unspecified Elimination Disorder.

### Sleep-Wake Disorders

Insomnia Disorder; Hypersomnolence Disorder; Narcolepsy; Obstructive Sleep Apnea Hypopnea; Central Sleep Apnea; Sleep-Related Hypoventilation; Circadian Rhythm Sleep-Wake Disorders; Non-Rapid Eye Movement Sleep Arousal Disorders; Nightmare Disorder; Rapid Eye Movement (REM) Sleep Behavior Disorder; Restless Legs Syndrome; Substance/Medication-Induced Sleep Disorder; Other Specified Insomnia Disorder; Unspecified Insomnia Disorder; Other Specified Hypersomnolence Disorder; Unspecified Hypersomnolence Disorder; Other Specified Sleep-Wake Disorder; Unspecified Sleep-Wake Disorder.

### Sexual Dysfunctions

Delayed Ejaculation; Erectile Disorder; Female Orgasmic Disorder; Female Sexual Interest/Arousal Disorder; Genito-Pelvic Pain/Penetration Disorder; Male Hypoactive Sexual Desire Disorder; Premature (Early) Ejaculation; Substance/Medication-Induced Sexual Dysfunction; Other Specified Sexual Dysfunction; Unspecified Sexual Dysfunction.

### Gender Dysphoria

Gender Dysphoria; Other Specified Gender Dysphoria; Unspecified Gender Dysphoria.

### Disruptive, Impulse-Control, and Conduct Disorders

Social Disorder; Oppositional Defiant Disorder; Intermittent Explosive Disorder; Conduct Disorder; Antisocial Personality Disorder; Pyromania; Kleptomania; Other Specified Disruptive, Impulse-Control, and Conduct Disorder; Unspecified Disruptive; Impulse-Control, and Conduct Disorder.

### Substance-Related and Addictive Disorders

Addiction; Alcohol Use Disorder; Alcohol Intoxication; Alcohol Withdrawal; Unspecified Alcohol-Related Disorder; Fetal Alcohol Syndrome; Caffeine Intoxication; Caffeine Withdrawal; Unspecified Caffeine-Related Disorder; Cannabis Use Disorder; Cannabis Intoxication; Cannabis Withdrawal; Unspecified Cannabis-Related Disorder; Phencyclidine Use Disorder; Other Hallucinogen Use Disorder; Phencyclidine Intoxication; Other Hallucinogen Intoxication; Hallucinogen Persisting Perception Disorder; Unspecified Phencyclidine-Related Disorder; Unspecified Hallucinogen-Related Disorder; Inhalant Use Disorder; Inhalant Intoxication; Unspecified Inhalant-Related Disorder; Opioid Use Disorder; Opioid Intoxication; Opioid Withdrawal; Unspecified Opioid-Related Disorder; Sedative, Hypnotic, or Anxiolytic Use Disorder; Sedative, Hypnotic, or Anxiolytic Intoxication; Sedative, Hypnotic, or Anxiolytic Withdrawal; Unspecified Sedative-, Hypnotic-, or Anxiolytic-Related Disorder; Stimulant Use Disorder; Stimulant Intoxication; Stimulant Withdrawal; Unspecified Stimulant-Related Disorder; Tobacco Use Disorder; Tobacco Withdrawal; Unspecified Tobacco-Related Disorder; Other (or Unknown) Substance Use Disorder; Other (or Unknown) Substance Intoxication; Other (or Unknown) Substance Withdrawal; Unspecified Other (or Unknown) Substance-Related Disorder; Gambling Disorder.

### Neurocognitive Disorders

Delirium; Other Specified Delirium; Unspecified Delirium; Major and Mild Neurocognitive Disorders; Major or Mild Neurocognitive Disorder Due to Alzheimer’s Disease; Major or Mild Frontotemporal Neurocognitive Disorder; Major or Mild Neurocognitive Disorder With Lewy Bodies; Major or Mild Vascular Neurocognitive Disorder; Major or Mild Neurocognitive Disorder Due to Traumatic Brain Injury; Substance/Medication-Induced Major or Mild Neurocognitive Disorder; Major or Mild Neurocognitive Disorder Due to HIV Infection; Major or Mild Neurocognitive Disorder Due to Prion Disease; Major or Mild Neurocognitive Disorder Due to Parkinson’s Disease; Major or Mild Neurocognitive Disorder Due to Huntington’s Disease; Major or Mild Neurocognitive Disorder Due to Another Medical Condition; Major or Mild Neurocognitive Disorder Due to Multiple Etiologies; Unspecified Neurocognitive Disorder.

### Personality Disorders

Dimensional Models for Personality Disorders; General Personality Disorder; Paranoid Personality Disorder; Schizoid Personality Disorder; Schizotypal Personality Disorder; Antisocial Personality Disorder; Borderline Personality Disorder; Histrionic Personality Disorder; Narcissistic Personality Disorder; Avoidant Personality Disorder; Dependent Personality Disorder; Obsessive-Compulsive Personality Disorder; Personality Change Due to Another Medical Condition; Other Specified Personality Disorder; Unspecified Personality Disorder.

### Paraphilic Disorders

Voyeuristic Disorder; Exhibitionistic Disorder; Frotteuristic Disorder; Sexual Masochism Disorder; Sexual Sadism Disorder; Pedophilic Disorder; Fetishistic Disorder; Transvestic Disorder; Other Specified Paraphilic Disorder; Unspecified Paraphilic Disorder.

### Other Mental Disorders

Other Specified Mental Disorder Due to Another Medical Condition; Unspecified Mental Disorder Due to Another Medical Condition; Other Specified Mental Disorder; Unspecified Mental Disorder.

### Medication-Induced Movement Disorders and Other Adverse Effects of Medication

Neuroleptic-Induced Parkinsonism Other Medication-Induced Parkinsonism; Neuroleptic Malignant Syndrome; Medication-Induced Acute Dystonia; Medication-Induced Acute Akathisia; Tardive Dyskinesia; Tardive Dystonia Tardive Akathisia; Medication-Induced Postural Tremor; Other Medication-Induced Movement Disorder; Antidepressant Discontinuation Syndrome; Other Adverse Effect of Medication.

### Symptoms of Neurological or Psychiatric Diseases and Disorders

Neurological or psychiatric diseases or disorders can manifest as a variety of symptoms. Non-limiting examples of symptoms of neurological or psychiatric diseases or disorders include symptoms such as apathy, depression, anxiety, cognitive impairment, psychosis, aggression, agitation, impulse control disorders, sleep disorders, elevated or irritable mood, hyperactivity, grandiosity, decreased need for sleep, racing thoughts, and in some cases, psychosis, anhedonia, sad mood, hopelessness, poor self-esteem, diminished concentration and lethargy, amyotrophic lateral sclerosis, primary lateral sclerosis, progressive muscular atrophy, progressive bulbar (atrophy) palsy, pseudobulbar palsy spinal muscular atrophy diseases (e.g., SMA type I, also called Werdnig-Hoffmann disease, SMA type II, SMA type III, also called Kugelberg-Welander disease, and Kennedy Disease, also called progressive spinobulbar muscular atrophy), Hallervorden-Spatz disease, Seitelberger disease (Infantile Neuroaxonal Dystrophy), adrenoleukodystrophy, Alexander Disease, autosomal dominant cerebellar ataxia (ADCA), pure autonomic failure (Bradbury-Eggleston Syndrome), CADASIL Syndrome, and neuronal ceroids lipofuscinose disorders such as Batten Disease (Spielmeyer-Vogt-Sjögren)); senile dementia, Early Onset Alzheimer’s Disease, Alzheimer’s type dementia, cognition, memory loss, amnesia/amnestic syndrome, disturbances of consciousness, coma, lowering of attention, speech disorder, agnosia, aphasia, apraxia, Mild Cognitive Impairment (MCI), benign forgetfulness, mild neurocognitive disorder, major neurocognitive disorder, neurocognitive disorder due to disease (e.g., Huntington’s Disease, Parkinson’s disease, Prion Disease, Traumatic Brain Injury, HIV or AIDS), Binswanger’s Disease (subcortical leukoencephalopathy), and Capgras Syndrome; or any other symptoms associated with a neurological or psychiatric disease or disorder disclosed herein.

### Pharmaceutical Compositions

In certain embodiments, provided herein is a composition (e.g., a pharmaceutical composition) comprising a compound described herein and a pharmaceutically acceptable excipient or carrier. In some embodiments, provided herein is a method of treating metabolic disorders in a subject in need thereof in a subject, comprising administering an effective amount of a compound or a pharmaceutical composition described herein. Examples of carriers and excipients are well known to those skilled in the art and are described in detail in, e.g., Ansel, Howard C., et al., Ansel’s Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present disclosure or pharmaceutical composition thereof) or aid in the manufacturing of the pharmaceutical product (i.e., medicament).

Compositions of the present disclosure may be administered orally, parenterally, by inhalation, topically, rectally, nasally, buccally, sublingually, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer’s solution and isotonic sodium chloride solution. In addition, sterile, fixed oils are conventionally employed as a solvent or suspending medium. Pharmaceutically acceptable compositions of this disclosure may be orally administered in any orally acceptable dosage form including capsules, tablets, aqueous suspensions or solutions.

The amount of compounds of the present disclosure that may be combined with the carrier materials to produce a composition in a single dosage form will vary depending upon a variety of factors, including the host treated and the particular mode of administration. It should also be understood that a specific dosage and treatment regimen for any particular subject will depend upon a variety of factors, including the activity of the specific compound employed; the age, body weight, general health, sex, and/or diet of the subject; time of administration, rate of excretion, drug combination, and the judgment of the treating physician and the severity of the particular disease being treated. The amount of a compound of the present disclosure in the composition will also depend upon the particular compound in the composition.

The compounds and compositions of the disclosure are preferably formulated in dosage unit form for ease of administration and uniformity of dosage. The expression “dosage unit form” as used herein refers to a physically discrete unit of agent appropriate for the subject to be treated. It will be understood, however, that the total daily usage of the compounds and compositions of the present disclosure will be decided by the attending physician within the scope of sound medical judgment.

### Combination Therapy

In some embodiments, compounds disclosed herein provide a method of treating a metabolic disorder described herein, comprising administering to a compound disclosed herein to a patient in need thereof in conjunction with one or more therapies. In some embodiments, the one or more therapies is an adjustment to diet routine of the patient, an adjustment to exercise routine of the patient, or a pharmaceutical agent.

In some embodiments, compounds disclosed herein provide a method of treating a metabolic disorder described herein, comprising administering a compound disclosed herein to a patient in need thereof in conjunction with one or more pharmaceutical agents. Suitable pharmaceutical agents that may be used in combination with the compounds of the present disclosure include anti-obesity agents (including appetite suppressants), anti-diabetic agents, antihyperglycemic agents, lipid lowering agents, anti-hypertensive agents, anti-Parkinson’s drugs, anti-Alzheimer’s drugs, anti-depressants, anti-psychotics, anti-ischemics, CNS depressants, anticholinergics, nootropics, epilepsy medication, attention (e.g., ADD/ADHD) medications, sleep-promoting medications, wakefulness-promoting medications, and pain medications.

Suitable lipid lowering agents that can be used in conjunction with compounds of the present disclosure include, but are not limited to, bile acid sequestrants, HMG-CoA reductase inhibitors, HMG-CoA synthase inhibitors, cholesterol absorption inhibitors, acyl coenzyme A-cholesterol acyl transferase (ACAT) inhibitors, CETP inhibitors, squalene synthetase inhibitors, PPAR-alpha agonists, FXR receptor modulators, LXR receptor modulators, lipoprotein synthesis inhibitors, renin-angiotensin system inhibitors, PPAR-delta partial agonists, bile acid reabsorption inhibitors, PPAR-gamma agonists, triglyceride synthesis inhibitors, microsomal triglyceride transport inhibitors, transcription modulators, squalene epoxidase inhibitors, low density lipoprotein receptor inducers, platelet aggregation inhibitors, 5-LO or FLAP inhibitors, niacin, and niacin-bound chromium.

Suitable anti-hypertensive agents that can be used in conjunction with compounds of the present disclosure include, but are not limited to, diuretics, beta-adrenergic blockers, calcium channel blockers, angiotensin converting enzyme (ACE) inhibitors, neutral endopeptidase inhibitors, endothelin antagonists, vasodilators, angiotensin II receptor antagonists, alpha/beta adrenergic blockers, alpha 1 blockers, alpha 2 agonists, aldosterone inhibitors, mineralocorticoid receptor inhibitors, renin inhibitors, and angiopoietin 2 binding agents.

Suitable anti-diabetic agents that can be used in conjunction with compounds of the present disclosure include, but are not limited to, other acetyl-CoA carboxylase (ACC) inhibitors, DGAT-1 inhibitors, AZD7687, LCQ908, DGAT-2 inhibitors, monoacylglycerol O-acyltransferase inhibitors, PDE-10 inhibitors, AMPK activators, sulfonylureas (e.g., acetohexamide, chlorpropamide, diabinese, glibenclamide, glipizide, glyburide, blimipiride, gliclazide, glipentide, gliquidone, glisolamide, tolazamide, tolbutamide), meglitinides, alpha-amylase inhibitors (e.g., tendamistat, treastatin, AL-3688), alpha-glucoside hydrolase inhibitors (e.g., acarbose), alpha-glucosidase inhibitors (e.g., adiposine, camiglibose, emiglitate, miglitol, voglibose, pradimicin-Q, sarbostatin), PPAR-gamma agonists (e.g., balaglitazone, ciglitazone, darglitazone, englitazone, isaglitazone, pioglitazone, rosiglitazone, troglitazone), PPAR-alpha/gamma agonists (e.g., CLX-0940, GW-1536, GW-1929, GW-2433, KRP-297, L-796449, LR-90, MK-0767, SB-219994), biguanides (e.g., metformin, buformin), GLP-1 modulators (exendin-3, exendin-4), liraglutide, albiglutide, exenatide (Byetta), taspoglutide, lixisenatide, dulaglutide, semaglutide, N,N-9924, TTP-054, PTP-1B inhibitors (trodusquemine, hyrtiosal extract), SIRT-1 inhibitors (e.g., resveratrol, GSK2245840, GSK184072), DPP-IV inhibitors (e.g., sitagliptin, vildagliptin, alogliptin, dutogliptin, linagliptin, saxagliptin), insulin secretagogues, fatty acid oxidation inhibitors, A2 antagonists, JNK inhibitors, glucokinase activators (e.g., TTP-399, TTP-355, TTP-547, AZD1656, ARRY403, MK-0599, TAK-329, AZD5658, GKM-001), insulin, insulin mimetics, glycogen phosphorylase inhibitors (e.g., GSK1362885), VPAC2 receptor agonists, SGLT2 inhibitors (dapagliflozin, canagliflozin, BI-10733, tofogliflozin, ASP-1941, THR1474, TS-071, ISIS388626, LX4211), glucagon receptor modulators, GPR119 modulators (e.g., MBX-2982, GSK1292263, APD597, PSN821), FGF21 derivatives, TGR5 (GPBAR1) receptor agonists (e.g., INT777), GPR40 agonists (e.g., TAK-875), GPR120 agonists, nicotinic acid receptor (HM74A) activators, SGLT1 inhibitors (e.g., GSK1614235), carnitine palmitoyl transferase enzyme inhibitors, fructose 1,6-diphosphatase inhibitors, aldose reductase inhibitors, mineralocorticoid receptor inhibitors, TORC2 inhibitors, CCR2 inhibitors, CCR5 inhibitors, PKC (e.g., PKC-alpha, PKC-beta, PKC-gamma) inhibitors, fatty acid synthetase inhibitors, serine palmitoyl transferase inhibitors, GPR81 modulators, GPR39 modulators, GPR43 modulators, GPR41 modulators, GPR105 modulators, Kv1.3 inhibitors, retinol binding protein 4 inhibitors, glucocorticoid receptor modulators, somatostatin receptor (e.g., SSTR1, SSTR2, SSTR3, SSTR5) inhibitors, PDHK2 inhibitors, PDHK4 inhibitors, MAP4K4 inhibitors, IL1-beta modulators, and RXR-alpha modulators.

Suitable anti-obesity agents include but are not limited to, 11-beta-hydroxysteroid dehydrogenase 1 inhibitors, stearoyl-CoA desaturase (SCD-1) inhibitors, MCR-4 agonists, CCK-A agonists, monoamine reuptake inhibitors (e.g., sibutramine), sympathomimetic agents, beta-3-adrenergic receptor agonists, dopamine receptor agonists (e.g., bromocriptine), melanocyte-stimulating hormone and analogs thereof, 5-HT_{2C} agonists (e.g., lorcaserin/Belviq), melanin concentrating hormone antagonists, leptin, leptin analogs, leptin agonists, galanin antagonists, lipase inhibitors (e.g., tetrahydrolipstatin/Orlistat), anorectic agents (e.g., bombesin agonists), NPY antagonists (e.g., velneperit), PYY₃₋₃₆ (and analogs thereof), BRS3 modulators, opioid receptor mixed antagonists, thyromimetic agents, dehydroepiandrosterone, glucocorticoid agonists or antagonists, orexin antagonists, GLP-1 agonists, ciliary neurotrophic factors (e.g., Axokine), human agouti-related protein (AGRP) inhibitors, H3 antagonists or inverse agonists, neuromedin U agonists, MTP/ApoB inhibitors (e.g., gut-selective MTP inhibitors such as dirlotapide, JTT130, Usistapide, SLX4090), MetAp2 inhibitors (e.g., ZGN-433), agents with mixed modulatory activity at two or more of glucagon, GIP, and GLP1 receptors (e.g., MAR-701, ZP2929), norepinephrine reuptake inhibitors, opioid antagonists (e.g., naltrexone), CB1 receptor antagonists or inverse agonists, ghrelin agonists or antagonists, oxyntomodulin and analogs thereof, monoamine uptake inhibitors (e.g., tesofensine), and combination agents (e.g., buproprion plus zonisamide (Empatic), pramlintide plus metreleptin, buproprion plus naltrexone (Contrave), phentermine plus topiramate (Qsymia).

In some embodiments, the anti-obesity agents used in combination with compounds of the disclosure are selected from gut-selective MTP inhibitors (e.g., dirlotapide, mitratapide, implitapide, R56918), CCK-A agonists, 5-HT_{2C} agonists (e.g., lorcaserin/Belviq), MCR4 agonists, lipase inhibitors (e.g., Cetilistat), PYY₃₋₃₆ (including analogs and PEGylated analogs thereof), opioid antagonists (e.g., naltrexone), oleoyl estrone, obinepitide, pramlintide, tesofensine, leptin, bromocriptine, orlistat, AOD-9604, and sibutramine.

Suitable anti-Parkinson’s drugs include dopamine replacement therapy (e.g., L-DOPA, carbidopa, COMT inhibitors such as entacapone or tolcapone), dopamine agonists (e.g., D1 agonists, D2 agonists, mixed D1/D2 agonists, bromocriptine, pergolide, cabergoline, ropinirole, pramipexole, piribedil, or apomorphine in combination with domperidone), histamine H2 antagonists, monoamine oxidase inhibitors (such as selegiline, rasagiline, safinamideand tranylcypromine), certain atypical antipsychotics such as pimavanserin (a non-dopaminergic atypical antipsychotic and inverse agonist of the serotonin 5-HT_{2A} receptor), and amantadine.

In some embodiments, compounds of the disclosure can be used in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexyl)hydrochloride, COMT inhibitors such as entacapone or tolcapone, MAO A/B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexole are commonly used in a non-salt form.

Suitable anti-Alzheimer’s drugs include beta-secretase inhibitors, gamma-secretase inhibitors, cholinesterase inhibitors such as donepezil, galantamine or rivastigmine, HMG-CoA reductase inhibitors, NSAID’s including ibuprofen, vitamin E, and anti-amyloid antibodies. In some embodiments, an anti-Alzheimer’s drug is memantine.

Suitable anti-depressants and anti-anxiety agents include norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT1A agonists or antagonists, especially 5-HT1A partial agonists, and corticotropin releasing factor (CRF) antagonists.

Specific suitable anti-depressant and anti-anxiety agents include amitriptyline, clomipramine, doxepin, imipramine and trimipramine; amoxapine, desipramine, citalopram, escitalopram, maprotiline, nortriptyline and protriptyline; fluoxetine, fluvoxamine, paroxetine and sertraline; isocarboxazid, phenelzine, tranylcypromine and selegiline; moclobemide: venlafaxine; desvenlafaxine, duloxetine; aprepitant; bupropion, vilazodone, mirtazapine, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, reboxetine, vortioxetine, clorazepate, and ketamine and pharmaceutically acceptable salts thereof. In some embodiments, suitable anti-depressant and anti-anxiety agents are tianeptine, or pharmaceutically acceptable salts thereof.

Suitable anti-psychotic and mood stabilizer agents include D2 antagonists, 5HT2A antagonists, atypical antipsychotics, lithium, and anticonvulsants.

Specific suitable anti-psychotic and mood stabilizer agents include chlorpromazine, fluphenazine, haloperidol, amisulpride, perphenazine, thioridazine, trifluoperazine, aripiprazole, asenapine, clozapine, olanzapine, paliperidone, brexpiprazole, paliperidone, cariprazine, pimavanserin, illoperidone, lumateperone, MIN-101, quetiapine, risperidone, ziprasidone, lurasidone, flupentixol, levomepromazine, pericyazine, perphenazine, pimozide, prochlorperazine, zuclopenthixol, olanzapine and fluoxetine, lithium, carbamazepine, lamotrigine, valproic acid, iloperidone, thiothixene, gabapentin, tiagabine and pharmaceutically acceptable salts thereof.

Suitable epilepsy medications include levetiracetam, oxcarbazepine, clobazam, retigabine, zonisamide, felbamate, esclicarbazepine acetate, lacosamide, carbamazepine, tiagabine, methsuximide, progabide, valproic acid, lamotrigine, brivaracetam, rufinamide, topiramate and perampanel.

Suitable attention medications include methyl phenidate, atomoxetine, guanfacine, D-amphetamine, lisdexamphetamine, methylamphetamine, and clonidine.

Suitable sleep-promoting medications include ramelteon, triazolam, zopiclone, eszopiclone, zolpidem, temazepam, and trazodone.

Suitable wakefulness-promoting medications include Modafinil, D-Amphetamine, caffeine, and armodafinil.

Suitable pain medications include dextromethorphan, tapentadol, buprenorphine, codeine, fentanyl, hydrocodone, hydromorphone, morphine, naloxegol, oxycodone, tramadol, gabapentil, difluprednate, pregabalin, acetyl salicyclic acid, bromfenac, diclofenac, diflunisal, indomethacin, ketorolac, meoxican, and naproxen.

In some embodiments, compounds and compositions disclosed herein may be used in combination with other therapies. Suitable therapies include psychotherapy, cognitive behavioral therapy, electroconvulsive therapy, transcranial magnetic stimulation, vagus nerve stimulation, and deep-brain stimulation.

Compound 1 is an antipsychotic. It is known that antipsychotics can be used to treat psychoses including schizophrenia, schizoaffective disorder, bipolar disorder, delusional disorder, schizophrenia spectrum disorder, schizophrenia negative symptoms, attenuated psychosis syndrome, prodromal schizophrenia, delusional disorder, psychosis, psychotic disorder, and major depression. Antipsychotics are also administered to relieve psychoses, the neuropsychiatric symptoms including delusions, hallucinations, aggressiveness, agitation, and delirium agitation associated with Parkinson’s disease, Alzheimer’s disease, amyotrophic lateral sclerosis, cognition loss, memory loss, amnesia/amnestic syndrome, epilepsy, disturbances of consciousness, coma, lowering of attention, speech disorder, Lennox syndrome, hyperkinetic syndrome, senile dementia, Alzheimer’s type dementia, and neurodevelopmental disorder. Compound 1 can be administered to treat conditions or symptoms treated by typical or atypical antipsychotics which act on the D2 receptor. Compound 1 has the advantage of not causing the adverse events associated with these typical and atypical antipsychotics.

Compound 1 can also be administered to reverse the adverse metabolic events caused by the administration of typical and atypical antipsychotics such as undesired weight gain and elevated glucose levels including type 2 diabetes and simultaneously treat the psychotic condition.

### EXAMPLES

The compounds of the present disclosure can be prepared in a number of ways known to one skilled in the art of organic synthesis in view of the methods, reaction schemes and examples provided herein, and international patent publications WO2011/069063 (and corresponding U.S. Pat. no. 8,710,245), WO2019/161238 (and corresponding U.S. Pat. 10,815,249), WO2018/151861 (and corresponding U.S. Pat. 11,129,807), WO2019/161236, WO2020/118032, and international patent application no. PCT/US2021/026953, each of which is incorporated by reference in their entirety.

The compounds of the present disclosure can be synthesized using the methods described below, together with synthetic methods known in the art of synthetic organic chemistry, or by variations thereon, as appreciated by those skilled in the art. Preferred methods include, but are not limited to, those described and/or referenced herein. The reactions are performed in a solvent or solvent mixture appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the disclosure. Examples are depicted with relative stereochemistry except where specifically stated otherwise.

The starting materials are generally available from commercial sources such as Sigma Aldrich or other commercial vendors, or are prepared as described or referenced in this disclosure, or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), Larock, R.C., Comprehensive Organic Transformations, 2^{nd} ed., Wiley-VCH Weinheim, Germany (1999), or Beilsteins Handbuch der organischen Chemie, 4, Aufl. ed. Springer-Verlag, Berlin, including supplements (also available via the Beilstein online database).

For illustrative purposes, the reaction schemes depicted or referenced herein provide potential routes for synthesizing the compounds of the present disclosure as well as key intermediates. Those skilled in the art will appreciate that other synthetic routes may be used to synthesize the compounds of the present disclosure. Although specific starting materials and reagents are depicted in the schemes, other starting materials and reagents can be easily substituted to provide a variety of derivatives and/or reaction conditions. In addition, many of the compounds prepared by the methods described or referenced herein can be further modified in light of this disclosure using conventional chemistry well known to those skilled in the art.

### Non-Clinical/Clinical Studies

Exemplary compounds disclosed herein were tested in non-clinical animal models as well as human clinical study.

### Example 1: Oral Glucose Tolerance Test (oGGT) Studies

### Example 1.1: Effect of Compound 1 on Glucose Tolerance in Lean Male C57BL/6J Mice

Compound 1 was investigated to explore the effects of administration of Compound 1 on glucose control in lean male C57BL/6J mice. An acute oral glucose tolerance test (OGTT) was performed following an overnight fast with Compound 1 being administered at four doses (0.3, 1, 3 and 10 mg/kg po) 30 minutes prior to the glucose load. A vehicle control group was included in the study design. In addition, the GLP 1 receptor agonist, exendin-4 (1 µg/kg ip) was utilised as a positive control, with this group being dosed 10 minutes prior to the glucose load. Blood samples were taken at various timepoints during the test and subsequently assessed for plasma glucose and insulin content.

### Materials and Methods

Animals and Animal Husbandry: Sixty-two (62) lean male C57BL/6J JAX mice (2 spares; 10 weeks of age; 22-27 g; as close to 25 g as possible) were purchased from Charles River, UK, Limited. Mice were weighed on the day of arrival (Thursday) and singly housed upon arrival and throughout the study in polypropylene cages on a normal phase 12 h light-dark cycle (lights on 07:00). Cages contained sawdust, red acrylic house, red acrylic tunnel, sizzlenest and nestlet at a temperature of 21±4° C. Upon arrival, mice were weighed and given wet mash to aid recovery from transport. Mice were weighed the following day and on the Monday of the following week. Relative humidity (RH) was typically 55±15% with prolonged periods below 40%RH or above 70%RH avoided as detailed in the UK Code of Practice. Low RH in the procedure room (average 35.91% or higher) was recorded on 4 days during the acclimatization phase and was largely driven by external conditions and not believed to have affected the study. Animals had free access to standard maintenance diet (Envigo Teklad 2018) and tap water ad libitum for the duration of the study unless otherwise stated.

Experimental Procedures: Animals underwent a three-day handling phase, which commenced five days after arrival. On the final day of handling the mice were dosed orally once with vehicle. At the start of the handling phase, animals were weighed (to the nearest 0.1 g using an electronic top-pan balance) and allocated into 6 weight-matched treatment groups by a Statistician. On the day of baseline dosing, mice were fasted for approximately 16 hours to a timed schedule, beginning at 16:45. The mice were placed into individual clean cages with access to water, but not food. The following morning the mice were moved to a separate room which was maintained with the same environmental conditions and underwent an OGTT. The groups for the study were: Group A - Vehicle (n = 10); Group B - Exendin-4 (1 µg/kg ip) (n = 10); Group C - Compound 1 (0.3 mg/kg po) (n = 10); Group D - Compound 1 (1 mg/kg po) (n = 10); Group E - Compound 1 (3 mg/kg po) (n = 10); Group F -Compound 1 (10 mg/kg po) (n = 10).

Following the overnight fast, a baseline blood sample (B1; 30 µl) was taken from all animals prior to vehicle or drug treatment. Five minutes later, the mice in groups A, C, D, E and F were dosed by the oral route (30 minutes pre-glucose). Twenty minutes later (10 minutes prior to the glucose load), mice in group B were dosed by the intraperitoneal route with exendin-4. All mice were given an oral glucose load of D-glucose at a dose of 2.0 g/kg (400 mg/mL solution; dose volume 5 mL/kg). Further blood samples (30 µl) were taken 3 minutes before glucose administration (B2) and at 15 (20 µl), 30 (20 µl), 60 (25 µl) and 120 (25 µl) minutes post glucose administration. All blood samples were collected into lithium heparinised tubes (Sarstedt Microvette CB300LH) and plasma separated by centrifugation to produce a single aliquot of plasma which was frozen (-80° C.) and subsequently assayed for glucose (Thermoelectron infinity glucose reagent, TR15421) and insulin (Alpco mouse ultrasensitive insulin kit 80 INSMSU-E10).

Upon completion of the OGTT, all animals were humanely killed by a Schedule 1 method (exposure to a rising concentration of CO₂ with confirmation of death by cervical dislocation). A terminal blood sample was taken from groups A, C, D, E and F only by cardiac puncture into a lithium heparin-coated tube (Sarstedt CB300LH) and the plasma separated by centrifugation. A single plasma aliquot (approximately 100 µl) was stored frozen prior to shipment of the compound to Charles River Laboratories, USA.

### Results

All animals were in good condition throughout the test and no adverse effects were evident at any stage of the protocol. Plasma glucose peaked from a baseline (B1) concentration of 6.2 mM to 24 mM at 30 minutes post-glucose load, subsequently reducing to 10 mM at 120 minutes post-glucose in the vehicle-control animals (Table 1.1.1). Exendin-4 (1 µg/kg ip) treatment 10 minutes before the glucose load produced a statistically significant (p<0.01) reduction in plasma glucose at all timepoints post-glucose and similarly reduced overall AUC and AUCB2 (0-60 and 0-120 minutes) compared to vehicle-treated control animals (Table 1.1.1, Table 1.1.2, Table 1.1.3). The four doses of Compound 1 (0.3, 1, 3 and 10 mg/kg po) administered 30 minutes before the glucose load significantly and dose-dependently (p<0.001, cf. vehicle controls) decreased plasma glucose at 15 and 30 minutes post the glucose dose (Table 1.1.1). Only the 10 mg/kg dose significantly (p<0.01) reduced plasma glucose at the 60 minute time point compared to controls and an increase in plasma glucose was observed with both the 10 and 3 mg/kg po doses (p<0.01) in comparison to the vehicle-treated animals at the 120 minute timepoint (Table 1.1.1). These reductions in plasma glucose induced by Compound 1 resulted in a statistically significant (p<0.05) and dose-dependent decrease in AUC and AUCB2 (both 0-60; Table 1.1.2, Table 1.1.3). Due to the trend for increases in plasma glucose to be observed at the 120 minute time point, only the highest dose of Compound 1 (10 mg/kg po) had a significant effect (reduction; p<0.01) on total AUC(0-120 minutes) (Table 1.1.2, Table 1.1.3).

Compound 1 had a similar profile on plasma insulin as it did on plasma glucose. Specifically, at the doses tested, plasma insulin was significantly (p<0.05) and dose-dependently reduced compared to vehicle-treated control counterparts both 15 minutes after the glucose load and in all (trend in total 0-120) AUC calculations (Table 1.1.4, Table 1.1.5, Table 1.1.6). Although not as marked as that observed for glucose (and not statistically significant) there was a trend for plasma insulin to be elevated above control levels at the 120 minute time point (Table 1.1.4).

TABLE 1.1.1 TABLE 1

Plasma glucose (mM)

| Treatment | n | Mean | SEM | % of vehicle | p |
|---|---|---|---|---|---|
| Pre-treatment baseline (B1) | | | | | |
| All treatments | 60 | 6.28 | 0.00 | | |
| Post-treatment baseline (B2) | | | | | |
| Vehicle po | 10 | 9.57 | 0.34 | | |
| Exendin-4 1 µg/kg ip | 10 | 10.66 | 0.45 | 111.4 | 0.194 |
| Compound 1 - 0.3 mg/kg po | 10 | 6.75 | 0.58 | 70.6 | 0.111 |
| Compound 1 - 1 mg/kg po | 10 | 9.25 | 0.87 | 96.7 | 0.111 |
| Compound 1 - 3 mg/kg po | 10 | 8.83 | 0.63 | 92.4 | 0.111 |
| Compound 1 - 10 mg/kg po | 10 | 8.57 | 0.44 | 89.6 | 0.111 |
| 15 minutes | | | | | |
| Vehicle po | 10 | 22.94 | 0.67 | | |
| Exendin-4 1 µg/kg ip | 10 | 15.13 | 0.58 | 65.9 | <0.001*** |
| Compound 1 - 0.3 mg/kg po | 10 | 19.39 | 0.83 | 84.5 | 0.010* |
| Compound 1 - 1 mg/kg po | 10 | 15.84 | 0.77 | 69.1 | <0.001*** |
| Compound 1 - 3 mg/kg po | 10 | 13.82 | 0.68 | 60.2 | <0.001*** |
| Compound 1 - 10 mg/kg po | 10 | 12.42 | 0.39 | 54.2 | <0.001*** |
| 30 minutes | | | | | |
| Vehicle po | 10 | 24.02 | 1.26 | | |
| Exendin-4 1 µg/kg ip | 10 | 12.81 | 0.86 | 53.3 | <0.001*** |
| Compound 1 - 0.3 mg/kg po | 10 | 21.40 | 1.10 | 89.1 | 0.098 |
| Compound 1 - 1 mg/kg po | 10 | 18.68 | 0.81 | 77.8 | <0.001*** |
| Compound 1 - 3 mg/kg po | 10 | 18.63 | 0.84 | 77.5 | <0.001*** |
| Compound 1 - 10 mg/kg po | 10 | 15.11 | 0.64 | 62.9 | <0.001*** |

60 minutes

| | | | | | |
|---|---|---|---|---|---|
| Vehicle po | 10 | 14.65 | 1.70 | | |
| Exendin-4 1 µg/kg ip | 10 | 8.56 | 0.76 | 58.4 | 0.002** |
| Compound 1 - 0.3 mg/kg po | 10 | 13.15 | 1.31 | 89.8 | 0.830 |
| Compound 1 - 1 mg/kg po | 10 | 14.36 | 1.52 | 98.1 | 0.830 |
| Compound 1 - 3 mg/kg po | 10 | 12.18 | 1.50 | 83.1 | 0.338 |
| Compound 1 - 10 mg/kg po | 10 | 9.35 | 1.03 | 63.8 | 0.009** |

120 minutes

| | | | | | |
|---|---|---|---|---|---|
| Vehicle po | 10 | 9.97 | 0.38 | | |
| Exendin-4 1 µg/kg ip | 10 | 7.46 | 0.41 | 74.8 | 0.003** |
| Compound 1 - 0.3 mg/kg po | 10 | 9.76 | 0.86 | 97.9 | >0.999 |
| Compound 1 - 1 mg/kg po | 10 | 10.63 | 0.47 | 106.6 | 0.589 |
| Compound 1 - 3 mg/kg po | 9 | 12.94 | 0.78 | 129.7 | 0.010** |
| Compound 1 - 10 mg/kg po | 10 | 15.29 | 1.80 | 153.3 | <0.001*** |

Means were back-transformed and adjusted for differences between the treatment groups in bleeding order, baseline body weight and pre-treatment baseline (B1) glucose. Standard errors of the mean (SEM) were calculated from the residuals of the statistical model. Analysis was by robust regression of log transformed data. Comparisons to vehicle were by multiple t test for Exendin-4 and by Williams’ test for Compound 1. *p<0.05, **p<0.01, ***p<0.001.

TABLE 1.1.2

Plasma glucose AUC (mM.hr)

| Treatment | n | Mean | SEM | % of vehicle | p |
|---|---|---|---|---|---|
| Area under curve (0-60 minutes) | | | | | |
| Vehicle po | 10 | 19.73 | 0.93 | | |
| Exendin-4 1 µg/kg ip | 10 | 12.14 | 0.62 | 61.5 | <0.001*** |
| Compound 1 - 0.3 mg/kg po | 10 | 16.95 | 0.62 | 85.9 | 0.009** |
| Compound 1 - 1 mg/kg po | 10 | 15.74 | 0.67 | 79.8 | <0.001*** |
| Compound 1 - 3 mg/kg po | 10 | 14.64 | 0.66 | 74.2 | <0.001*** |
| Compound 1 - 10 mg/kg po | 10 | 12.51 | 0.48 | 63.4 | <0.001*** |

Area under curve (0-120 minutes)

| | | | | | |
|---|---|---|---|---|---|
| Vehicle po | 10 | 32.14 | 1.84 | | |
| Exendin-4 1 g/kg ip | 10 | 20.44 | 1.03 | 63.6 | <0.001*** |
| Compound 1 - 0.3 mg/kg po | 10 | 28.47 | 1.20 | 88.6 | 0.185 |
| Compound 1 - 1 mg/kg po | 10 | 28.24 | 1.39 | 87.9 | 0.185 |
| Compound 1 - 3 mg/kg po | 9 | 28.37 | 1.60 | 88.3 | 0.185 |
| Compound 1 - 10 mg/kg po | 10 | 24.86 | 1.60 | 77.4 | 0.004** |

Means were back-transformed and adjusted for differences between the treatment groups in bleeding order, baseline day 1 body weight and day 1 pre-treatment baseline (B1) glucose. Standard errors of the mean (SEM) were calculated from the residuals of the statistical model. Analysis was by robust regression of log transformed data. Comparisons to vehicle were by multiple t test for Exendin-4 and by Williams’ test for Compound 1 -. **p<0.01, ***p<0.001.

TABLE 1.1.3

Plasma glucose AUCB2 (mM.hr)

| Treatment | n | Mean | SEM | Difference from vehicle | p |
|---|---|---|---|---|---|
| AUC above baseline (0-60 minutes) | | | | | |
| Vehicle po | 10 | 9.81 | 1.16 | | |
| Exendin-4 1 µg/kg ip | 10 | 1.49 | 0.77 | -8.32 | <0.001*** |
| Compound 1 - 0.3 mg/kg po | 10 | 10.12 | 0.93 | 0.31 | >0.999 |
| Compound 1 - 1 mg/kg po | 10 | 6.95 | 1.10 | -2.86 | 0.011* |
| Compound 1 - 3 mg/kg po | 10 | 6.32 | 0.54 | -3.49 | 0.002** |
| Compound 1 - 10 mg/kg po | 10 | 3.89 | 0.62 | -5.93 | <0.001*** |

AUC above baseline (0-120 minutes)

| | | | | | |
|---|---|---|---|---|---|
| Vehicle po | 10 | 12.41 | 2.27 | | |
| Exendin-4 1 µg/kg ip | 10 | -0.58 | 1.28 | -12.99 | <0.001*** |
| Compound 1 - 0.3 mg/kg po | 10 | 14.52 | 1.70 | 2.11 | >0.999 |
| Compound 1 - 1 mg/kg po | 10 | 11.12 | 1.99 | -1.29 | 0.841 |
| Compound 1 - 3 mg/kg po | 9 | 11.39 | 1.38 | -1.02 | 0.841 |
| Compound 1 - 10 mg/kg po | 10 | 7.79 | 1.78 | -4.62 | 0.097 |

Means were adjusted for differences between the treatment groups in bleeding order, baseline body weight and pre-treatment baseline (B1) glucose. Standard errors of the mean (SEM) were calculated from the residuals of the statistical model. Analysis was by robust regression of untransformed data. Comparisons to vehicle were by multiple t test for Exendin-4 and by Williams’ test for Compound 1 -. *p<0.05, **p<0.01, ***p<0.001.

TABLE 1.1.4

Plasma insulin (ng/mL)

| Treatment | n | Mean | SEM | % of vehicle | p |
|---|---|---|---|---|---|
| Pre-treatment baseline Day 1 (B1) | | | | | |
| All treatments | 60 | 0.13 | 0.00 | | |
| Post-treatment baseline (B2) | | | | | |
| Vehicle po | 10 | 0.15 | 0.04 | | |
| Exendin-4 1 µg/kg ip | 10 | 0.34 | 0.04 | 221.2 | 0.004** |
| Compound 1 - 0.3 mg/kg po | 10 | 0.15 | 0.04 | 99.4 | >0.999 |
| Compound 1 - 1 mg/kg po | 10 | 0.15 | 0.04 | 97.6 | >0.999 |
| Compound 1 - 3 mg/kg po | 10 | 0.18 | 0.05 | 119.9 | 0.630 |
| Compound 1 - 10 mg/kg po | 10 | 0.19 | 0.03 | 125.7 | 0.513 |
| 15 minutes | | | | | |
| Vehicle po | 10 | 1.25 | 0.12 | | |
| Exendin-4 1 µg/kg ip | 10 | 1.92 | 0.40 | 154.4 | 0.010* |
| Compound 1 - 0.3 mg/kg po | 10 | 0.86 | 0.05 | 68.7 | 0.025* |
| Compound 1 - 1 mg/kg po | 10 | 0.65 | 0.06 | 52.0 | <0.001*** |
| Compound 1 - 3 mg/kg po | 10 | 0.60 | 0.10 | 48.2 | <0.001*** |
| Compound 1 - 10 mg/kg po | 10 | 0.60 | 0.05 | 48.5 | <0.001*** |
| 30 minutes | | | | | |
| Vehicle po | 10 | 1.00 | 0.05 | | |
| Exendin-4 1 µg/kg ip | 10 | 1.10 | 0.13 | 110.3 | 0.520 |
| Compound 1 - 0.3 mg/kg po | 10 | 1.04 | 0.12 | 104.4 | >0.999 |
| Compound 1 - 1 mg/kg po | 10 | 0.81 | 0.06 | 81.0 | 0.198 |
| Compound 1 - 3 mg/kg po | 10 | 0.74 | 0.10 | 74.3 | 0.066 |
| Compound 1 - 10 mg/kg po | 10 | 0.52 | 0.05 | 52.1 | <0.001*** |
| 60 minutes | | | | | |
| Vehicle po | 10 | 0.51 | 0.03 | | |
| Exendin-4 1 µg/kg ip | 10 | 0.37 | 0.06 | 73.1 | 0.010** |
| Compound 1 - 0.3 mg/kg po | 10 | 0.59 | 0.05 | 116.1 | >0.999 |
| Compound 1 - 1 mg/kg po | 10 | 0.50 | 0.04 | 98.2 | >0.999 |
| Compound 1 - 3 mg/kg po | 10 | 0.47 | 0.04 | 91.2 | 0.549 |
| Compound 1 - 10 mg/kg po | 10 | 0.38 | 0.02 | 75.0 | 0.019* |
| 120 minutes | | | | | |
| Vehicle po | 10 | 0.42 | 0.06 | | |
| Exendin-4 1 µg/kg ip | 10 | 0.27 | 0.04 | 63.5 | 0.026* |
| Compound 1 - 0.3 mg/kg po | 10 | 0.40 | 0.05 | 95.4 | >0.999 |
| Compound 1 - 1 mg/kg po | 10 | 0.43 | 0.06 | 102.0 | >0.999 |
| Compound 1 - 3 mg/kg po | 9 | 0.52 | 0.06 | 121.9 | 0.428 |
| Compound 1 - 10 mg/kg po | 10 | 0.61 | 0.06 | 143.6 | 0.090 |

Means were back-transformed and adjusted for differences between the treatment groups in bleeding order, baseline body weight and pre-treatment baseline (B1) insulin. Standard errors of the mean (SEM) were calculated from the residuals of the statistical model. Analysis was by robust regression of log transformed data. Comparisons to vehicle were by multiple t test for Exendin-4 and by Williams’ test for Compound 1 -. *p<0.05, **p<0.01, ***p<0.001.

TABLE 1.1.5

Plasma insulin (ng/mL.hr) - Area Under Curve (AUC)

| Treatment | n | Mean | SEM | % of vehicle | p |
|---|---|---|---|---|---|
| Area under curve (0-60 minutes) | | | | | |
| Vehicle po | 10 | 0.85 | 0.03 | | |
| Exendin-4 1 µg/kg ip | 10 | 1.06 | 0.07 | 125.7 | 0.053 |
| Compound 1 - 0.3 mg/kg po | 10 | 0.79 | 0.06 | 93.2 | 0.542 |
| Compound 1 - 1 mg/kg po | 10 | 0.63 | 0.04 | 73.8 | 0.012* |
| Compound 1 - 3 mg/kg po | 10 | 0.61 | 0.07 | 71.5 | 0.006** |
| Compound 1 - 10 mg/kg po | 10 | 0.48 | 0.03 | 56.2 | <0.001*** |

Area under curve (0-120 minutes)

| | | | | | |
|---|---|---|---|---|---|
| Vehicle po | 10 | 1.33 | 0.05 | | |
| Exendin-4 1 µg/kg ip | 10 | 1.48 | 0.11 | 110.9 | 0.342 |
| Compound 1 - 0.3 mg/kg po | 10 | 1.30 | 0.09 | 97.4 | 0.805 |
| Compound 1 - 1 mg/kg po | 10 | 1.11 | 0.09 | 83.3 | 0.110 |
| Compound 1 - 3 mg/kg po | 9 | 1.06 | 0.11 | 79.4 | 0.051 |
| Compound 1 - 10 mg/kg po | 10 | 0.98 | 0.06 | 73.6 | 0.007** |

Means were back-transformed and adjusted for differences between the treatment groups in bleeding order, baseline body weight and pre-treatment baseline (B1) insulin. Standard errors of the mean (SEM) were calculated from the residuals of the statistical model. Analysis was by robust regression of log transformed data. Comparisons to vehicle were by multiple t test for Exendin-4 and by Williams’ test for Compound 1 -. *p<0.05, **p<0.01, ***p<0.001.

TABLE 1.1.6

Plasma insulin (ng/mL.hr) - AUC above baseline

| Treatment | n | Mean | SEM | Difference from vehicle | p |
|---|---|---|---|---|---|
| AUC above baseline (0-60 minutes) | | | | | |
| Vehicle po | 10 | 0.70 | 0.04 | | |
| Exendin-4 1 µg/kg ip | 10 | 0.72 | 0.04 | 0.02 | 0.772 |
| Compound 1 - 0.3 mg/kg po | 10 | 0.62 | 0.05 | -0.08 | 0.234 |
| Compound 1 - 1 mg/kg po | 10 | 0.46 | 0.04 | -0.24 | <0.001*** |
| Compound 1 - 3 mg/kg po | 10 | 0.43 | 0.05 | -0.27 | <0.001*** |
| Compound 1 - 10 mg/kg po | 10 | 0.28 | 0.02 | -0.42 | <0.001*** |

AUC above baseline (0-120 minutes)

| | | | | | |
|---|---|---|---|---|---|
| Vehicle po | 10 | 1.04 | 0.06 | | |
| Exendin-4 1 µg/kg ip | 10 | 0.76 | 0.08 | -0.28 | 0.010* |
| Compound 1 - 0.3 mg/kg po | 10 | 0.99 | 0.08 | -0.05 | 0.635 |
| Compound 1 - 1 mg/kg po | 10 | 0.77 | 0.07 | -0.26 | 0.016* |
| Compound 1 - 3 mg/kg po | 9 | 0.75 | 0.07 | -0.28 | 0.012* |
| Compound 1 - 10 mg/kg po | 10 | 0.59 | 0.06 | -0.44 | <0.001*** |

Means were adjusted for differences between the treatment groups in bleeding order, baseline body weight and pre-treatment baseline (B1) insulin. Standard errors of the mean (SEM) were calculated from the residuals of the statistical model. Analysis was by robust regression of untransformed data. Comparisons to vehicle were by multiple t test for Exendin-4 and by Williams’ test for Compound 1 -. *p<0.05, ***p<0.001.

### Example 1.2: Effect of Compound 1 on Glucose Tolerance in Diabetic Db/Db Mice

Compound 1 was investigated to explore the effects of administration of Compound 1 on glucose control in male db/db mice. An acute oral glucose tolerance test (OGTT) was performed following an overnight fast with Compound 1 being administered at four doses (0.3, 1, 3 and 10 mg/kg po) 30 minutes prior to the glucose load. A vehicle control group was included in the study design. In addition, the GLP 1 receptor agonist, exendin-4 (300 µg/kg ip) was utilised as a positive control, with this group being dosed 10 minutes prior to the glucose load. Blood samples were taken at various timepoints during the test and subsequently assessed for plasma glucose and insulin content.

### Materials and Methods

Animals and Animal Husbandry: Sixty-two (62) male db/db mice (2 spares; 6-7 weeks of age) were purchased from Charles River, Italy, Limited. Mice were weighed on the day of arrival (Thursday) and group housed in polypropylene cages on a normal phase 12 h light-dark cycle (lights on 07:00). Cages contained sawdust, red house, red tunnel, Sizzlenest and nestlet at a temperature of 24±2° C. Upon arrival, mice were weighed and given wet mash to aid recovery from transport. Mice were weighed the following day and on the Monday of the following week. Relative humidity (RH) was 55±15%. Animals had free access to standard maintenance diet (Envigo Teklad 2018) and tap water ad libitum for the duration of the study unless otherwise stated.

Experimental Procedures: Animals underwent a three-day handling phase, which commenced five days after arrival. On the final day of handling the mice were dosed orally once with vehicle. At the start of the handling phase, animals were weighed (to the nearest 0.1 g using an electronic top-pan balance) and allocated into 6, weight matched treatment groups by a Statistician. On the day of baseline dosing, mice were fasted for approximately 16 hours to a timed schedule, beginning at 16:45. The mice were placed into individual clean cages with access to water, but not food. The following morning the mice were moved to a separate room which was maintained with the same environmental conditions and underwent an OGTT. The groups for the study were: Group A - Vehicle (n = 10); Group B - Exendin-4 (1 µg/kg ip) (n = 10); Group C - Compound 1 (0.3 mg/kg po) (n = 10); Group D - Compound 1 (1 mg/kg po) (n = 10); Group E - Compound 1 (3 mg/kg po) (n = 10); Group F -Compound 1 (10 mg/kg po) (n = 10).

Following the overnight fast, a baseline blood sample (B1; 30 µl) was taken from all animals prior to vehicle or drug treatment. Five minutes later, the mice in groups A, C, D, E and F were dosed by the oral route (30 minutes pre-glucose). Twenty minutes later (10 minutes prior to the glucose load), mice in group B were dosed by the intraperitoneal route with exendin-4. All mice were given an oral glucose load of D-glucose at a dose of 2.0 g/kg (400 mg/mL solution; dose volume 5 mL/kg). Further blood samples (30 µl) were taken 3 minutes before glucose administration (B2) and at 15 (20 µl), 30 (20 µl), 60 (25 µl) and 120 (25 µl) minutes post glucose administration. All blood samples were collected into lithium heparinised tubes (Sarstedt Microvette CB300LH) and plasma separated by centrifugation to produce a single aliquot of plasma which was frozen (-80° C.) and subsequently assayed for glucose (Thermoelectron infinity glucose reagent, TR15421) and insulin (Alpco mouse ultrasensitive insulin kit 80 INSMSU-E10).

Upon completion of the OGTT, all animals were humanely killed by a Schedule 1 method (exposure to a rising concentration of CO₂ with confirmation of death by cervical dislocation). A terminal blood sample was taken from groups A, C, D, E and F only by cardiac puncture into a lithium heparin-coated tube (Sarstedt CB300LH) and the plasma separated by centrifugation. A single plasma aliquot (approximately 100 µl) was stored frozen prior to shipment of the compound to Charles River Laboratories, USA.

### Results

All animals were in good condition throughout the test and no adverse effects were evident at any stage of the protocol. Plasma glucose peaked from a baseline (B1) concentration of 9.42 mM to 54.93 mM at 30 minutes post-glucose load, subsequently reducing to 20.44 mM at 120 minutes post-glucose in the vehicle-control animals (Table 1.2.1). Exendin-4 (300 µg/kg ip) treatment 10 minutes before the glucose load produced a statistically significant (p<0.001) reduction in plasma glucose at all timepoints post-glucose and similarly reduced overall AUC and AUCB2 (0-60 and 0-120 minutes) compared to vehicle-treated control animals (Table 1.2.1, Table 1.2.2, Table 1.2.3).

The three doses of Compound 1 (1, 3 and 10 mg/kg po) administered 30 minutes before the glucose load significantly (p<0.001, cf. vehicle controls) decreased plasma glucose at 15 minutes and the four doses of Compound 1 (0.3, 1, 3 and 10 mg/kg po) decreased plasma glucose at 30 minutes post the glucose dose (p<0.01, cf. vehicle controls) (Table 1.2.1). A later rebound increase in plasma glucose was observed at the 120 minute time point at 1, 3 and 10 mg/kg po dose (p<0.001) in comparison to the vehicle-treated animals (Table 1.2.1). These reductions in plasma glucose induced by Compound 1 resulted in a statistically significant (p<0.05) decrease in AUC and AUCB2 (both 0-60; Table 1.2.2, Table 1.2.3).

Compound 1 had a similar profile on plasma insulin as it did on plasma glucose. Specifically, at 1, 3 and 10 mg/kg po, plasma insulin was significantly (p<0.01) and dose-dependently reduced compared to vehicle-treated control counterparts both 15 and 60 minutes after the glucose load and by 10 mg/kg po (p<0.05) at 30 minutes post glucose load. In the AUC calculations, doses of 3 and 10 mg/kg po were significantly (p<0.01) and dose-dependently reduced (Table 1.2.4, Table 1.2.5). In the AUCB2 at the 1, 3 and 10 mg/kg po dose, plasma insulin was significantly (p<0.05) reduced compared to vehicle treated control counterparts at 0-60 minutes and, in the case of the 3 and 10 mg/kg po doses, at 60-120 minutes post glucose load (Table 1.2.6).

TABLE 1.2.1

Plasma glucose (mM)

| Treatment | n | Mean | SEM | % of vehicle | p |
|---|---|---|---|---|---|
| Pre-treatment baseline (B1) | | | | | |
| All treatments | 60 | 9.42 | 0.00 | | |

Post-treatment baseline (B2)

| | | | | | |
|---|---|---|---|---|---|
| Vehicle po | 10 | 17.80 | 0.62 | | |
| Exendin-4 300 µg/kg ip | 10 | 17.81 | 0.78 | 100.0 | 0.995 |
| Compound 1 - 0.3 mg/kg po | 10 | 17.18 | 0.86 | 96.5 | >0.999 |
| Compound 1 - 1 mg/kg po | 10 | 18.32 | 0.85 | 102.9 | 0.868 |
| Compound 1 - 3 mg/kg po | 10 | 18.25 | 0.91 | 102.5 | 0.868 |
| Compound 1 - 10 mg/kg po | 10 | 19.96 | 1.19 | 112.1 | 0.121 |
| Vehicle po | 10 | 51.61 | 2.32 | | |
| Exendin-4 300 µg/kg ip | 10 | 20.37 | 2.06 | 39.5 | <0.001*** |
| Compound 1 - 0.3 mg/kg po | 10 | 45.05 | 2.08 | 87.3 | 0.051 |
| Compound 1 - 1 mg/kg po | 10 | 40.33 | 1.66 | 78.1 | <0.001*** |
| Compound 1 - 3 mg/kg po | 10 | 32.62 | 1.59 | 63.2 | <0.001*** |
| Compound 1 - 10 mg/kg po | 10 | 35.94 | 1.26 | 69.6 | <0.001*** |
| Vehicle po | 10 | 54.93 | 2.61 | | |
| Exendin-4 300 µg/kg ip | 10 | 19.07 | 2.21 | 34.7 | <0.001*** |
| Compound 1 - 0.3 mg/kg po | 10 | 46.08 | 1.32 | 83.9 | 0.004** |
| Compound 1 - 1 mg/kg po | 10 | 45.78 | 0.90 | 83.3 | 0.003** |
| Compound 1 - 3 mg/kg po | 10 | 38.07 | 1.21 | 69.3 | <0.001*** |
| Compound 1 - 10 mg/kg po | 10 | 40.39 | 1.22 | 73.5 | <0.001*** |
| Vehicle po | 10 | 39.22 | 3.66 | | |
| Exendin-4 300 µg/kg ip | 10 | 12.91 | 1.13 | 32.9 | <0.001*** |
| Compound 1 - 0.3 mg/kg po | 10 | 36.82 | 1.36 | 93.9 | >0.999 |
| Compound 1 - 1 mg/kg po | 10 | 38.69 | 1.25 | 98.6 | >0.999 |
| Compound 1 - 3 mg/kg po | 10 | 35.07 | 1.35 | 89.4 | >0.999 |
| Compound 1 - 10 mg/kg po | 10 | 41.26 | 1.07 | 105.2 | 0.638 |
| Vehicle po | 10 | 20.44 | 2.15 | | |
| Exendin-4 300 µg/kg ip | 10 | 10.71 | 0.58 | 52.4 | <0.001*** |
| Compound 1 - 0.3 mg/kg po | 10 | 21.59 | 1.33 | 105.6 | 0.499 |
| Compound 1 - 1 mg/kg po | 10 | 27.20 | 0.96 | 133.1 | <0.001*** |
| Compound 1 - 3 mg/kg po | 10 | 27.59 | 1.02 | 135.0 | <0.001*** |
| Compound 1 - 10 mg/kg po | 10 | 37.75 | 1.05 | 184.7 | <0.001*** |

Means were back-transformed and adjusted for differences between the treatment groups in bleeding order, baseline body weight and pre-treatment baseline (B1) glucose. Standard errors of the mean (SEM) are calculated from the residuals of the statistical model. Analysis was by robust regression of log transformed data. Comparisons to vehicle were by multiple t test for Exendin-4 and by Williams’ test for Compound 1 -. **p<0.01, ***p<0.001.

TABLE 1.2.2

Plasma glucose AUC (mM.hr)

| Treatment | n | Mean | SEM % of vehicle | p | |
|---|---|---|---|---|---|
| Area under curve (0-60 minutes) | | | | | |
| Vehicle po | 10 | 45.79 | 2.16 | | |
| Exendin-4 300 µpg/kg ip | 10 | 17.77 | 1.61 | 38.8 | <0.001*** |
| Compound 1 - 0.3 mg/kg po | 10 | 39.78 | 1.22 | 86.9 | 0.033* |
| Compound 1 - 1 mg/kg po | 10 | 39.13 | 1.03 | 85.5 | 0.020* |
| Compound 1 - 3 mg/kg po | 10 | 33.73 | 1.10 | 73.7 | <0.001*** |
| Compound 1 - 10 mg/kg po | 10 | 37.05 | 0.98 | 80.9 | <0.001*** |
| Area under curve (0–120 minutes) | | | | | |
| Vehicle po | 10 | 76.29 | 4.88 | | |
| Exendin-4 300 µg/kg ip | 10 | 30.22 | 2.45 | 39.6 | <0.001*** |
| Compound 1 - 0.3 mg/kg po | 10 | 69.62 | 2.40 | 91.3 | 0.332 |
| Compound 1 - 1 mg/kg po | 10 | 71.87 | 1.89 | 94.2 | 0.332 |
| Compound 1 - 3 mg/kg po | 10 | 65.29 | 2.15 | 85.6 | 0.332 |
| Compound 1 - 10 mg/kg po | 10 | 76.47 | 1.87 | 100.2 | 0.332 |

Means were back-transformed and adjusted for differences between the treatment groups in bleeding order, baseline body weight and day 1 pre-treatment baseline (B1) glucose. Standard errors of the mean (SEM) were calculated from the residuals of the statistical model. Analysis was by robust regression of log transformed data. Comparisons to vehicle were by multiple t test for Exendin-4 and by Williams’ test for Compound 1 -. *p<0.05, ***p<0.001.

TABLE 1.2.3

Plasma glucose (mM.hr) - AUC above baseline

| Treatment | n | Mean | SEM | Difference from vehicle | p |
|---|---|---|---|---|---|
| AUC above baseline (0-60 minutes) | | | | | |
| Vehicle po | 10 | 27.40 | 1.73 | | |
| Exendin-4 300 µg/kg ip | 10 | -1.09 | 1.16 | -28.49 | <0.001*** |
| Compound 1 - 0.3 mg/kg po | 10 | 22.68 | 1.12 | -4.73 | 0.012* |
| Compound 1 - 1 mg/kg po | 10 | 20.57 | 0.68 | -6.84 | <0.001*** |
| Compound 1 - 3 mg/kg po | 10 | 15.51 | 1.24 | -11.89 | <0.001*** |
| Compound 1 - 10 mg/kg po | 10 | 16.84 | 0.95 | -10.56 | <0.001*** |

AUC above baseline (0-120 minutes)

| | | | | | |
|---|---|---|---|---|---|
| Vehicle po | 10 | 39.96 | 4.66 | | |
| Exendin-4 300 µg/kg ip | 10 | -8.04 | 2.07 | -47.99 | <0.001*** |
| Compound 1 - 0.3 mg/kg po | 10 | 34.90 | 2.03 | -5.06 | 0.181 |
| Compound 1 - 1 mg/kg po | 10 | 34.65 | 1.64 | -5.31 | 0.181 |
| Compound 1 - 3 mg/kg po | 10 | 28.44 | 2.29 | -11.51 | 0.052 |
| Compound 1 - 10 mg/kg po | 10 | 35.89 | 2.53 | -4.07 | 0.052 |

Means were adjusted for differences between the treatment groups in bleeding order, baseline body weight and pre-treatment baseline (B1) glucose. Standard errors of the mean (SEM) were calculated from the residuals of the statistical model. Analysis was by robust regression of untransformed data. Comparisons to vehicle were by multiple t test for Exendin-4 and by Williams’ test for Compound 1 -. *p<0.05, ***p<0.001.

TABLE 1.2.4

Plasma insulin (ng/mL)

| Treatment | n | Mean | SEM | % of vehicle | p |
|---|---|---|---|---|---|
| Pre-treatment baseline Day 1 (B1) | | | | | |
| All treatments | 60 | 3.61 | 0.00 | | |

Post-treatment baseline (B2)

| Vehicle po | 10 | 3.96 | 0.27 | | |
|---|---|---|---|---|---|
| Exendin-4 300 µg/kg ip | 10 | 8.99 | 0.47 | 227.2 | <0.001*** |
| Compound 1 - 0.3 mg/kg po | 10 | 3.63 | 0.32 | 91.8 | 0.710 |
| Compound 1 - 1 mg/kg po | 10 | 3.62 | 0.28 | 91.5 | 0.710 |
| Compound 1 - 3 mg/kg po | 10 | 3.88 | 0.17 | 98.1 | 0.710 |
| Compound 1 - 10 mg/kg po | 10 | 3.03 | 0.27 | 76.5 | 0.021* |
| 15 minutes | | | | | |
| Vehicle po | 10 | 6.43 | 0.80 | | |
| Exendin-4 300 µg/kg ip | 10 | 11.33 | 2.21 | 176.4 | 0.001** |
| Compound 1 - 0.3 mg/kg po | 10 | 5.75 | 0.47 | 89.5 | 0.503 |
| Compound 1 - 1 mg/kg po | 10 | 4.11 | 0.33 | 63.9 | 0.010* |
| Compound 1 - 3 mg/kg po | 10 | 3.38 | 0.27 | 52.6 | <0.001*** |
| Compound 1 - 10 mg/kg po | 10 | 2.81 | 0.19 | 43.7 | <0.001*** |
| 30 minutes | | | | | |
| Vehicle po | 10 | 4.12 | 0.46 | | |
| Exendin-4 300 µg/kg ip | 10 | 9.42 | 0.96 | 228.5 | <0.001*** |
| Compound 1 - 0.3 mg/kg po | 10 | 3.96 | 0.41 | 96.2 | 0.804 |
| Compound 1 - 1 mg/kg po | 10 | 3.61 | 0.29 | 87.6 | 0.484 |
| Compound 1 - 3 mg/kg po | 10 | 3.24 | 0.26 | 78.6 | 0.162 |
| Compound 1 - 10 mg/kg po | 10 | 2.73 | 0.28 | 66.3 | 0.013* |
| 60 minutes | | | | | |
| Vehicle po | 10 | 4.25 | 0.39 | | |
| Exendin-4 300 µg/kg ip | 10 | 8.95 | 0.47 | 210.5 | <0.001*** |
| Compound 1 - 0.3 mg/kg po | 10 | 3.76 | 0.33 | 88.4 | 0.310 |
| Compound 1 - 1 mg/kg po | 10 | 2.96 | 0.27 | 69.7 | 0.005** |
| Compound 1 - 3 mg/kg po | 10 | 2.94 | 0.26 | 69.2 | 0.004** |
| Compound 1 - 10 mg/kg po | 10 | 2.76 | 0.19 | 64.9 | <0.001*** |
| 120 minutes | | | | | |
| Vehicle po | 10 | 4.14 | 0.40 | | |
| Exendin-4 300 µg/kg ip | 10 | 9.87 | 0.65 | 238.7 | <0.001*** |
| Compound 1 - 0.3 mg/kg po | 10 | 3.87 | 0.26 | 93.7 | 0.566 |
| Compound 1 - 1 mg/kg po | 10 | 3.54 | 0.20 | 85.6 | 0.465 |
| Compound 1 - 3 mg/kg po | 10 | 3.55 | 0.15 | 85.9 | 0.465 |
| Compound 1 - 10 mg/kg po | 10 | 4.09 | 0.28 | 98.9 | 0.465 |

Means were back-transformed and adjusted for differences between the treatment groups in bleeding order, baseline body weight and pre-treatment baseline (B1) insulin. Standard errors of the mean (SEM) were calculated from the residuals of the statistical model. Analysis was by robust regression of log transformed data. Comparisons to vehicle were by multiple t test for Exendin-4 and by Williams’ test for Compound 1 -. *p<0.05, **p<0.01, ***p<0.001.

TABLE 1.2.5

Plasma insulin (ng/mL.hr) - Area Under Curve (AUC)

| Treatment | n | Mean | SEM | % of vehicle | p |
|---|---|---|---|---|---|
| Area under curve (0-60 minutes) | | | | | |
| Vehicle po | 10 | 4.66 | 0.43 | | |
| Exendin-4 300 µg/kg ip | 10 | 9.93 | 0.87 | 213.0 | <0.001*** |
| Compound 1 - 0.3 mg/kg po | 10 | 4.34 | 0.38 | 93.1 | 0.598 |
| Compound 1 - 1 mg/kg po | 10 | 3.62 | 0.25 | 77.7 | 0.080 |
| Compound 1 - 3 mg/kg po | 10 | 3.28 | 0.23 | 70.3 | 0.013* |
| Compound 1 - 10 mg/kg po | 10 | 2.79 | 0.21 | 59.9 | <0.001*** |

Area under curve (0-120 minutes)

| | | | | | |
|---|---|---|---|---|---|
| Vehicle po | 10 | 8.67 | 0.75 | | |
| Exendin-4 300 µg/kg ip | 10 | 19.34 | 1.13 | 223.1 | <0.001*** |
| Compound 1 - 0.3 mg/kg po | 10 | 8.10 | 0.66 | 93.4 | 0.543 |
| Compound 1 - 1 mg/kg po | 10 | 6.94 | 0.46 | 80.0 | 0.061 |
| Compound 1 - 3 mg/kg po | 10 | 6.54 | 0.39 | 75.4 | 0.017* |
| Compound 1 - 10 mg/kg po | 10 | 6.24 | 0.42 | 72.0 | 0.006** |

Means were back-transformed and adjusted for differences between the treatment groups in bleeding order, baseline body weight and pre-treatment baseline (B1) insulin. Standard errors of the mean (SEM) were calculated from the residuals of the statistical model. Analysis was by robust regression of log transformed data. Comparisons to vehicle were by multiple t test for Exendin-4 and by Williams’ test for Compound 1 -. *p<0.05, **p<0.01, ***p<0.001.

TABLE 1.2.6

Plasma insulin (ng/mL.hr) - AUC above baseline

| Treatment | n | Mean | SEM | Difference from vehicle | p |
|---|---|---|---|---|---|
| AUC above baseline (0-60 minutes) | | | | | |
| Vehicle po | 10 | 0.97 | 0.38 | | |
| Exendin-4 300 µg/kg ip | 10 | 0.89 | 0.83 | -0.08 | 0.844 |
| Compound 1 - 0.3 mg/kg po | 10 | 0.67 | 0.12 | -0.30 | 0.462 |
| Compound 1 - 1 mg/kg po | 10 | 0.02 | 0.21 | -0.95 | 0.027* |
| Compound 1 - 3 mg/kg po | 10 | -0.53 | 0.21 | -1.50 | 0.001** |
| Compound 1 - 10 mg/kg po | 10 | -0.31 | 0.18 | -1.28 | 0.001** |

AUC above baseline (0-120 minutes)

| | | | | | |
|---|---|---|---|---|---|
| Vehicle po | 10 | 1.33 | 0.73 | | |
| Exendin-4 300 µg/kg ip | 10 | 0.98 | 1.08 | -0.35 | 0.680 |
| Compound 1 - 0.3 mg/kg po | 10 | 0.66 | 0.26 | -0.67 | 0.428 |
| Compound 1 - 1 mg/kg po | 10 | -0.23 | 0.44 | -1.56 | 0.083 |
| Compound 1 - 3 mg/kg po | 10 | -1.10 | 0.37 | -2.43 | 0.037* |
| Compound 1 - 10 mg/kg po | 10 | 0.01 | 0.41 | -1.32 | 0.037* |

Means were adjusted for differences between the treatment groups in bleeding order, baseline body weight and pre-treatment baseline (B1) insulin. Standard errors of the mean (SEM) were calculated from the residuals of the statistical model. Analysis was by robust regression of untransformed data. Comparisons to vehicle were by multiple t test for Exendin-4 and by Williams’ test for Compound 1 -. *p<0.05,**p<0.01.

### Example 2: Olanzapine-Induced Model of Body Weight Gain

### Example 2.1: Effect of Compound 1 and Olanzapine on Body Weight in Female Sprague Dawley Rats

Compound 1 was investigated to explore the effects of administration of Compound 1 once daily for a maximum of 15 days in female Sprague Dawley rats fed on a high-fat powdered diet. Body weight and food and water intake were assessed daily. Olanzapine was included as a reference comparator. This model is sensitive to the effects of olanzapine on body weight.

### Materials and Methods

Animals: Seventy-four (2 spares) female Sprague Dawley rats (weight range 200-300 g) were ordered from Charles River, Margate, Kent. Rats were singly housed in cages with sawdust bedding and enrichment at an ambient temperature of 21 ± 2° C. Upon arrival (Thursday), rats were weighed and given wet mash to aid recovery from transport. Rats were weighed the following day and on the Monday of the following week. Animals were maintained on a reverse-phase light-dark cycle (lights off for 8 hours from 10:00-18 :00 h) during which time the room was illuminated by red light. Rats were weighed on the Friday and Monday of the following week. Relative humidity was typically 55 ± 15% with prolonged periods below 40%RH or above 70%RH avoided as detailed in the UK Code of Practice. Except for 12 animals (Group F), all animals had free access to a high-fat powdered diet (VRF1 plus 20% lard). The 12 animals in Group F received Envigo Teklad 2018 maintenance diet in a powdered form. All rats had access to filtered water at all times. The diets were contained in glass feeding jars with aluminum lids (Solmedia Laboratory Suppliers. Romford. Essex). Each lid had a 3-4 cm hole cut in it to allow access to the food. Animals were accustomed to these conditions for at least two weeks before experimentation.

Experimental Procedure: Animals underwent a 3-day baseline run-in period during which time all rats were dosed once a day with vehicle. Dosing was timed to start at approximately 08:45 so that the mid-point was approximately the time of lights out. Animals, food and water were weighed at the time of dosing. Towards the end of the baseline period, animals were weighed (to the nearest 0.1 g using an electronic top-pan balance) and allocated by a statistician. The spare animals were humanely withdrawn from the study. Subsequently, rats were dosed once daily as follows: Group A - Vehicle (3 mL/kg po (n = 12); Group B - Olanzapine (3 mg/kg po) (n = 12); Group C - Compound 1 (1 mg/kg po) (n = 12); Group D - Compound 1 (3 mg/kg po) (n = 12); Group E - Compound 1 (10 mg/kg po) (n = 12); Group F - Vehicle (maintenance diet) (n = 12).

Dosing (in a randomized order) began at approximately 08:45 h each day (0 h), i.e., the mid-point of dosing is approximately at the time of lights out. Body weight, food and water intake was recorded daily at the time of dosing. All rats were observed before and after dosing and comments on condition were noted as appropriate. On the afternoon of Day 14 (approx. 16:00) animals were fasted to a timed schedule. The experiment ended the following morning on Day 15. On the morning of Day 15, final readings were undertaken 16 hour post-fast (i.e., body weight measurements). Animals were then dosed (as described). Four hours later, a blood sample (1 mL) was taken from the lateral tail vein into tubes containing lithium heparin. The blood was centrifuged at 2,400 g for 5 min at 4° C. to produce five aliquots of plasma which was stored frozen for option plasma analysis at Sygnature Discovery Ltd (glucose, insulin, triglycerides, cholesterol (HDL, LDL, total) and NEFA). Animals were then terminated by a Schedule 1 method (exposure to an increasing concentration of C02 with death confirmed by cervical dislocation). A terminal blood sample (circa 4 mL) was taken into an EDTA-coated tube and a single plasma aliquot (approximately 1 mL) was stored frozen (approx. -80° C.) in a clean aliquot tube. The liver was dissected and the caudate lobe removed. Both the caudate lobe and the remaining liver were weighed and stored frozen separately (on dry ice and then transferred to a freezer at approx. -80° C.). The caudate lobe underwent option further analysis of liver triglycerides.

### Results

Olanzapine (3 mg/kg/day, po) significantly increased body weight (Table 2.1.1) and food intake (Table 2.1.2) compared to vehicle treated rats. Initial effects were observed as early as 1-2 days following treatment. In contrast, Compound 1 (1, 3 and 10 mg/kg/day, po) significantly and dose-dependently reduced body weights and food intake compared to vehicle treatment. Importantly, following an initial decrease (days 1-4), body weights and food intake were maintained throughout the remainder of the treatment duration. In addition, body composition analysis revealed a significant and dose-dependent reduction in percent fat in animals treated with Compound 1 (Table 2.1.3). These preliminary results suggest a differentiated weight gain profile of Compound 1 compared to atypical antipsychotics. Potentially as a consequence of the body weight reduction, liver triglycerides levels were improved with Compound 1 (i.e., dose-dependent reduction compared to vehicle, Table 2.1.4). No statistically significant effects on liver triglycerides were observed with olanzapine.

TABLE 2.1.1

Changes in body weights (g)

| Time | Treatment | n | Mean | SEM | vs vehicle | |
|---|---|---|---|---|---|---|
| | | | | | Difference | p |
| Overall | Vehicle - 3 mL/kg po | 12 | 11.7 | 2.7 | | |
| (Day 1-14) | Olanzapine - 3 mg/kg po | 12 | 33.2 | 3.6 | 21.5 | <0.001*** |
| | Compound 1 - 1 mg/kg po | 12 | 4.0 | 1.7 | -7.7 | 0.033* |
| | Compound 1 - 3 mg/kg po | 12 | -1.9 | 1.4 | -13.6 | <0.001*** |
| | Compound 1 - 10 mg/kg po | 12 | -5.8 | 2.4 | -17.5 | <0.001*** |

Means are adjusted for differences between the treatment groups at baseline (Day 1). Standard errors of the mean (SEM) are calculated from the residuals of the statistical model. Multiple comparisons are by Williams’ test to compare Compound 1 - to vehicle and multiple t test to compare Olanzapine to vehicle. *p<0.05, **p<0.01, ***p<0.001.

TABLE 2.1.2

Daily food intake (g/day)

| Time | Treatment | n | Mean | SEM | Differenc e | % change | p |
|---|---|---|---|---|---|---|---|
| Overall | Vehicle - 3 mL/kg po | 12 | 14.5 | 0.5 | | | |
| (Day 1-13) | Olanzapine - 3 mg/kg po Compound 1 - 1 mg/kg po | 12 12 | 16.4 13.4 | 0.4 0.5 | 2.0 -1.1 | 13.6 -7.3 | 0.001** 0.073 |
| | Compound 1 - 3 mg/kg po | 12 | 12.4 | 0.2 | -2.1 | -14.4 | <0.001** |
| | | | | | | | * |
| | Compound 1 - 10 mg/kg po | 12 | 11.5 | 0.3 | -3.0 | -20.5 | <0.001** * |

Means are adjusted for differences between the treatment groups at baseline (average of Days -2 to 0).
Standard errors of the mean (SEM) are calculated from the residuals of the statistical model. Multiple comparisons are by Williams’ test to compare Compound 1 - to vehicle and multiple t test to compare Olanzapine to vehicle. *p<0.05, **p<0.01, ***p<0.001.

TABLE 2.1.3

Body composition results

| Treatment | n | Mean | SEM | vs vehicle | | |
|---|---|---|---|---|---|---|
| | | | | Difference | % change | p |
| Water in carcass (%) | | | | | | |
| Vehicle - 3 mL/kg po | 12 | 60.7 | 1.0 | | | |
| Olanzapine - 3 mg/kg po | 12 | 59.9 | 0.9 | -0.8 | -1.4 | 0.446 |
| Compound 1 - 1 mg/kg po | 12 | 62.2 | 0.6 | 1.5 | 2.5 | 0.166 |
| Compound 1 - 3 mg/kg po | 12 | 62.8 | 0.7 | 2.1 | 3.5 | 0.061 |
| Compound 1 - 10 mg/kg po | 12 | 64.4 | 0.5 | 3.6 | 6.0 | 0.001** |

Water per rat (g)

| | | | | | | |
|---|---|---|---|---|---|---|
| Vehicle - 3 mL/kg po | 12 | 160.7 | 2.7 | | | |
| Olanzapine - 3 mg/kg po | 12 | 166.5 | 1.9 | 5.8 | 3.6 | 0.083 |
| Compound 1 - 1 mg/kg po | 12 | 160.4 | 2.2 | -0.3 | -0.2 | 0.918 |
| Compound 1 - 3 mg/kg po | 12 | 159.0 | 2.5 | -1.7 | -1.0 | 0.818 |

TABLE 2.1.3

Body composition results

| Treatment | n | Mean | SEM | vs vehicle | | |
|---|---|---|---|---|---|---|
| | | | | Difference | % change | p |
| Compound 1 - 10 mg/kg po | 12 | 159.3 | 1.9 | -1.4 | -0.9 | 0.818 |
| Fat in carcass (%) | | | | | | |
| Vehicle - 3 mL/kg po | 12 | 15.6 | 1.4 | | | |
| Olanzapine - 3 mg/kg po | 12 | 16.9 | 1.1 | 1.4 | 8.8 | 0.308 |
| Compound 1 - 1 mg/kg po | 12 | 13.6 | 0.7 | -2.0 | -13.0 | 0.135 |
| Compound 1 - 3 mg/kg po | 12 | 12.6 | 0.9 | -2.9 | -18.9 | 0.035* |
| Compound 1 - 10 mg/kg po | 12 | 11.0 | 0.6 | -4.5 | -29.1 | 0.001** |
| Fat per rat (g) | | | | | | |
| Vehicle - 3 mL/kg po | 12 | 41.9 | 4.0 | | | |
| Olanzapine - 3 mg/kg po | 12 | 47.0 | 3.6 | 5.2 | 12.4 | 0.142 |
| Compound 1 - 1 mg/kg po | 12 | 35.4 | 1.8 | -6.4 | -15.4 | 0.069 |
| Compound 1 - 3 mg/kg po | 12 | 32.6 | 2.2 | -9.3 | -22.2 | 0.011* |
| Compound 1 - 10 mg/kg po | 12 | 27.8 | 1.5 | -14.1 | -33.7 | <0.001*** |
| Protein in carcass (%) | | | | | | |
| Vehicle - 3 mL/kg po | 12 | 20.84 | 0.37 | | | |
| Olanzapine - 3 mg/kg po | 12 | 20.46 | 0.36 | -0.38 | -1.8 | 0.278 |
| Compound 1 - 1 mg/kg po | 12 | 21.34 | 0.15 | 0.50 | 2.4 | 0.157 |
| Compound 1 - 3 mg/kg po | 12 | 21.63 | 0.27 | 0.78 | 3.8 | 0.031* |
| Compound 1 - 10 mg/kg po | 12 | 21.89 | 0.19 | 1.05 | 5.0 | 0.004** |
| Protein per rat (g) | | | | | | |
| Vehicle - 3 mL/kg po | 12 | 55.0 | 0.8 | | | |
| Olanzapine - 3 mg/kg po | 12 | 56.9 | 0.8 | 1.9 | 3.5 | 0.058 |
| Compound 1 - 1 mg/kg po | 12 | 54.9 | 0.5 | -0.2 | -0.3 | 0.889 |
| Compound 1 - 3 mg/kg po | 12 | 54.7 | 0.8 | -0.3 | -0.6 | 0.889 |
| Compound 1 - 10 mg/kg po | 12 | 54.1 | 0.6 | -0.9 | -1.7 | 0.443 |

Means are adjusted for Day 1 body weight differences between the treatment groups. Standard errors of the mean (SEM) are calculated from the residuals of the statistical model. Multiple comparisons are by Williams’ test to compare Compound 1 - to vehicle and multiple t test to compare Olanzapine to vehicle. *p<0.05, **p<0.01, ***p<0.001.

TABLE 2.1.4

Liver triglycerides

| Treatment | n | Mean | SEM | vs vehicle | |
|---|---|---|---|---|---|
| | | | | % | p |
| Tri glycerides (µmoles/g) | | | | | |
| Vehicle - 3 mL/kg po | 12 | 32.51 | 4.81 | | |
| Olanzapine - 3 mg/kg po | 12 | 25.07 | 5.06 | 77.1 | 0.244 |
| Compound 1 - 1 mg/kg po | 12 | 20.79 | 3.56 | 64.0 | 0.048* |
| Compound 1 - 3 mg/kg po | 12 | 18.01 | 2.19 | 55.4 | 0.011* |
| Compound 1 - 10 mg/kg po | 12 | 14.19 | 1.29 | 43.6 | <0.001*** |

TABLE 2.1.4

Liver triglycerides

| Treatment | n | Mean | SEM | vs vehicle | |
|---|---|---|---|---|---|
| Total triglycerides (µmoles) | | | | | |
| Vehicle (3 mL/kg po) | 12 | 265.60 | 36.29 | | |
| Olanzapine - 3 mg/kg po | 12 | 227.46 | 46.94 | 85.6 | 0.480 |
| Compound 1 - 1 mg/kg po | 12 | 170.40 | 30.19 | 64.2 | 0.046* |
| Compound 1 - 3 mg/kg po | 12 | 143.97 | 15.36 | 54.2 | 0.008** |
| Compound 1 - 10 mg/kg po | 12 | 117.77 | 10.41 | 44.3 | <0.001*** |

Means are back-transformed and adjusted for bleeding order and Day 1 body weight differences between the treatment groups. Standard errors of the mean (SEM) are calculated from the residuals of the statistical model. Multiple comparisons are by Williams’ test to compare Compound 1 - to vehicle and multiple t test to compare Olanzapine to vehicle. *p<0.05, **p<0.01, ***p<0.001.

### Example 2.2: Effect of Compound 1 on the Body Weight of Female Sprague Dawley Rats Previously Treated With Olanzapine

Compound 1 was investigated to explore the effects of administration of Compound 1 on the body weight and metabolic parameters of rats previously treated with olanzapine, including exploring whether administration of the test compound restores body weight to or beyond control levels. Animals received either vehicle or olanzapine once daily for a period of 7 days. An increase in body weight of the animals receiving olanzapine compared to vehicle-treated animals was shown. Subsequently, from Day 8 onwards, the olanzapine-treated animals either continued to receive olanzapine, were switched to vehicle, or were switched to Compound 1 (0.3, 1 and 3 mg/kg, po).

### Materials and Methods

Animals: Seventy-four (2 spares) female Sprague Dawley rats (weight range 200-250 g) were ordered from Charles River, Margate, Kent. Rats were singly housed in cages with sawdust bedding and enrichment at an ambient temperature of 21±2° C. Upon arrival (Thursday), rats were weighed and given wet mash to aid recovery from transport. Rats were weighed the following day and the Monday of the following week. Animals were maintained on a reverse-phase light-dark cycle (lights off for 8 h from 10:00-18:00 h) during which time the room was illuminated by red light. Rats were weighed on the Friday and Monday of the following week. Relative humidity was typically 55 ± 15% with prolonged periods below 40%RH or above 70%RH avoided as detailed in the UK Code of Practice. All animals had free access to a high-fat powdered diet (VRF1 plus 20% lard) and all rats had access to filtered water at all times. The diets were contained in glass feeding jars with aluminum lids (Solmedia Laboratory Suppliers, Romford, Essex). Each lid had a 3-4 cm hole cut in it to allow access to the food. Animals were accustomed to these conditions for at least two weeks before experimentation.

Experimental Procedures: Animals had a 3-day baseline run-in period during which time all rats were dosed once a day with vehicle. Dosing was timed to start at approximately 08:45 so that the mid-point was approximately the time of lights out. Animals, food and water were weighed at the time of dosing. Towards the end of this baseline period, animals were weighed (to the nearest 0.1 g using an electronic top-pan balance) and allocated into 2 weight-matched treatment groups by a Statistician. Subsequently, rats were dosed once daily as follows: Treatment 1 (po) Days 1-4 or 1-7: Group A - Vehicle (3 mL/kg) (n = 12); Groups B-F -Olanzapine (3 mg/kg) (n = 62).

Olanzapine (3 mg/kg po) has previously been shown in-house to increase body weight by 4-6% in this model. Dosing began at approximately 08:45 h each day (0 h), i.e., so the mid-point of dosing was approximately at the time of lights out. This strategy was taken to maximize the impact of the drugs on food intake. Dosing continued for 7 consecutive days and rats were weighed (to the nearest 0.1 g) every day at 0 h. Prior to and at the completion of each dosing session animals were examined and any overt behavioral/physiological effects or other relevant observations regarding the condition of the animals was recorded manually. From Day 4 onwards, body weight data was analyzed to assess whether a statistically significant increase in weight was evident with olanzapine compared to vehicle-treated controls fed the high-fat diet. When this reached statistical significance, rats in groups B-F were allocated further by a Statistician on the basis of available body weight as follows: Treatment 2: Group A - Vehicle (aqueous methyl cellulose, 1%, 3 mL/kg po) (n = 12); Group B - Olanzapine (3 mg/kg po) (n = 12); Group C -Compound 1 (0.3 mg/kg po) (n = 12); Group D - Compound 1 (1 mg/kg po) (n = 12); Group E -Compound 1 (3 mg/kg po) (n = 12); Group F - Vehicle (water, 3 mL/kg) (n = 12). Subsequently, from Day 8 onwards, animals either continued to receive olanzapine, or were switched to vehicle or Compound 1 (0.3, 1 and 3 mg/kg, po). Body intake and food intake were assessed daily.

Dosing began at approximately 08:45 h each day (0h), i.e., the mid-point of dosing was approximately at the time of lights out. Body weight, food and water intake was recorded daily at the time of dosing. All rats were observed before and after dosing and comments on condition were noted as appropriate. On the afternoon of Day 14 (approx. 16:00) animals were fasted to a timed schedule. The experiment ended the following morning on Day 15. On the morning of Day 15, final readings were undertaken 16 h post-fast (i.e., body weight measurements). Animals were then dosed (as described). Four hours later, two blood samples (20 µL and 1 mL) were taken from the lateral tail vein into tubes containing lithium heparin. The 20 µL sample was stored frozen (whole blood) prior to determination of HbAlc (Roche Tina-quant A2c Gen 3, Cobas C111). The larger blood sample were centrifuged at 2,400 g for 5 min at 4° C. to produce five aliquots of plasma which were stored frozen for optional plasma analysis at Sygnature Discovery Ltd (glucose, insulin, triglycerides, cholesterol (HDL, LDL, total) and NEFA). Animals were then terminated by a Schedule 1 method (exposure to an increasing concentration of CO₂ with death confirmed by cervical dislocation). A terminal blood sample (circa 4 mL) was taken into EDTA-coated tube and a single plasma aliquot (approximately 1 mL) was stored frozen (approx. -80° C.) in a clean aliquot tube. The liver was dissected and the caudate lobe removed. Both the caudate lobe and the remaining liver was weighed and stored frozen separately (on dry ice and then transferred to a freezer at approx. -80° C.). The caudate lobe underwent further analysis of liver triglycerides. Carcasses were stored frozen (approx. -20° C.) for potential body composition analysis. Metabolic parameters including fasting glucose and insulin levels, liver triglycerides and body fat composition were determined at the end of the study.

### Results

In line with previous publications, olanzapine (3 mg/kg/day, po) significantly increased body weight (Table 2.2.1) and food intake (Table 2.2.2) compared to vehicle treated rats. Initial effects were observed as early as 2-3 days following dosing. On day 8, animals either continued to receive olanzapine or were switched to vehicle or Compound 1 treatment. The switch to Compound 1 treatment reversed olanzapine-induced weight gain and food intake at all doses tested (0.3, 1 and 3 mg/kg, po). Importantly, a more rapid reversal of olanzapine-induced weight gain was seen in rats switched to Compound 1 treatment compared to vehicle. In addition, body composition analysis revealed a significant reduction in percent fat in animals switched to Compound 1 treatment compared to rats that continuously received olanzapine (Table 2.2.3). Liver triglycerides levels were decreased with Compound 1 but the reduction did not reach statistical significance (Table 2.2.4). These preliminary results suggest that Compound 1 reduces weight gain associated with prior antipsychotic drug treatment.

TABLE 2.2.1

Changes in body weights (g)

| Time | Treatment | n | Mean | SEM | vs Vehicle (A) | | vs Vehicle (F) | | vs Olanzapine (B) | |
|---|---|---|---|---|---|---|---|---|---|---|
| | | | | | Difference | p | Difference | p | Difference | p |
| Week 1 | Vehicle po | 12 | 12.41 | 2.04 | | | | | | |
| (Day 1-8) | Olanzapine 3 mg/kg po | 61 | 25.48 | 0.93 | 13.07 | <0.001*** | | | | |
| Week 2 | Vehicle (aqueous methyl cellulose) po | 12 | 9.15 | 2.00 | | | | | | |
| (Day 8-14) | Olanzapine - 3 mg/kg po | 12 | 16.70 | 1.91 | 7.55 | 0.006* * | | | | |
| | Compound 1 -- 0.3 mg/kg po | 12 | -1.19 | 1.91 | -10.34 | <0.001*** | -5.85 | 0.025* | -17.90 | <0.001*** |
| | Compound 1 -- 1 mg/kg po | 12 | -6.37 | 1.91 | -15.52 | <0.001*** | -11.03 | <0.001*** | -23.08 | <0.001*** |
| | Compound 1 - 3 mg/kg po | 12 | -5.31 | 1.91 | -14.46 | <0.001*** | -9.97 | <0.001*** | -22.02 | <0.001*** |
| | Vehicle (water) po | 13 | 4.65 | 1.83 | -4.50 | 0.098 | | | -12.05 | <0.001 *** |

Means are estimated from the mixed offsets model. Standard errors of the mean (SEM) are calculated from the residuals of tire statistical model. Compound 1 - was compared to Vehicle (A) and Olanzapine (B) by separate Dunnett’s tests and Vehicle (F) by Williams’ test. Other comparisons by multiple t test. *p<0.05, * *p<0.01, ***p<0.001.

TABLE 2.2.2

Averages of daily food intakes (g/day)

| Time | Treatment | n | Mean | SEM | vs Vehicle (A) | | vs Vehicle (F) | | vs Olanzapine (B) | |
|---|---|---|---|---|---|---|---|---|---|---|
| | | | | | Difference | p | Difference | p | Difference | p |
| Week 1 | Vehicle (aqueous methyl cellulose) po | 12 | 15.31 | 0.35 | | | | | | |
| (Day 1-7) | Olanzapine 3 mg/kg po | 61 | 17.21 | 0.18 | 1.90 | <0.001*** | | | | |
| Week 2 | Vehicle (aqueous methyl cellulose) po | 12 | 15.13 | 0.36 | | | | | | |
| (Day 8-13) | Olanzapine 3 mg/kg po | 12 | 16.73 | 0.32 | 1.60 | <0.001*** | | | | |
| | Compound 1 - 0.3 mg/kg po | 12 | 12.98 | 0.32 | -2.14 | <0.001*** | -1.89 | <0.001*** | -3.75 | <0.001*** |
| | Compound 1 - 1 mgkg po | 12 | 12.17 | 0.32 | -2.95 | <0.001*** | -2.70 | <0.001*** | -4.56 | <0.001*** |
| | Compound 1 - 3 mg/kg po | 12 | 12.41 | 0.32 | -2.71 | <0.001 *** | -2.46 | <0.001*** | -4.31 | <0.001*** |
| | Vehicle (water) po | 13 | 14.87 | 0.31 | -0.26 | 0.573 | | | -1.86 | <0.001*** |

Means are estimated from the mixed effects model. Standard errors of the mean (SEM) are calculated from the residuals of the statistical model. Compound 1 - was compared to Vehicle (A) and Olanzapine (B) by separate Dunnett’s tests and Vehicle (F) by Williams’ test. Other comparisons by multiple t test **p<0.01,***p<0.001.

TABLE 2.2.3

Body composition data

| Treatment | n | Mean | SEM | vs Vehicle (A) | | | vs Vehicle (F) | | vs Olanzapine (B) | |
|---|---|---|---|---|---|---|---|---|---|---|
| | | | | Difference | % change | p | Difference | p | Difference | p |
| Water in carcass (%) | | | | | | | | | | |
| Vehicle (aqueous methyl cellulose) po | 12 | 62.6 | 0.5 | | | | | | | |
| Olanzapine - 3 mg/kg po | 12 | 60.4 | 0.4 | -2.2 | -3.6 | 0.006** | | | | |
| Compound 1 - 0.3 mg/kg po | 12 | 63.1 | 0.5 | 0.6 | 0.9 | 0.816 | 0.6 | 0.585 | 2.8 | 0.002** |
| Compound 1 - 1 mg/kg po | 12 | 63.0 | 0.5 | 0.4 | 0.6 | 0.934 | 0.5 | 0.585 | 2.6 | 0.004** |
| Compound 1 - 3 mg/kg po | 12 | 63.1 | 0.7 | 0.5 | 0.8 | 0.866 | 0.6 | 0.585 | 2.7 | 0.003** |
| Vehicle (water) po | 13 | 62.5 | 0.6 | -0.1 | -0.1 | 0.913 | | | 2.1 | 0.007** |
| Water per rat (g) | | | | | | | | | | |
| Vehicle (aqueous methyl cellulose) po | 12 | 163.4 | 1.4 | | | | | | | |
| Olanzapine - 3 mg/kg po | 12 | 163.9 | 1.6 | 0.6 | 0.3 | 0.835 | | | | |
| Compound 1 - 0.3 mg/kg po | 12 | 165.2 | 2.2 | 1.9 | 1.1 | 0.835, | -2.0 | 0.469 | 1.3 | 0.934 |
| Compound 1 - 1 mg/kg po | 12 | 160.8 | 1.7 | -2.6 | -1.6 | 0.668 | -6.4 | 0.023* | -3.2 | 0.524 |
| Compound 1 - 3 mg/kg po | 12 | 160.5 | 2.3 | -2.9 | -1.8 | 0.595 | -6.7 | 0.018* | -3.4 | 0.456 |
| Vehicle (water) po | 13 | 167.2 | 2.1 | 3.8 | 2.3 | 0.159 | | | 3.3 | 0.229 |

Fat in carcass (%)

| | | | | | | | | | | |
|---|---|---|---|---|---|---|---|---|---|---|
| Vehicle (aqueous methyl cellulose) po | 12 | 13.4 | 0.6 | | | | | | | |
| Olanzapine - 3 mg/kg po | 12 | 15.8 | 0.6 | 2.4 | 18.3 | 0.016* | | | | |
| Compound 1 - 0.3 mg/kg po | 12 | 12.5 | 0.6 | -0.9 | -6.5 | 0.709 | -1.0 | 0.399 | -3.3 | |
| Compound 1 - 1 mg/kg po | 12 | 12.6 | 0.6 | -0.8 | -5.7 | 0.784 | -0.9 | 0.399 | -3.2 | 0.005** |
| Compound 1 - 3 mg/kg po | 12 | 12.4 | 0.7 | -0.9 | -7.0 | 0.663 | -1.1 | 0.350 | -3.4 | 0.003** |
| Vehicle (water) po | 13 | 13.5 | 0.7 | 0.1 | 1.0 | 0.893 | | | -2.3 | 0.020* |

Means are adjusted for Day 1 body weight differences between the treatment groups. Standard errors of the mean (SEM) are calculated from the residuals of the statistical model. Compound 1 - was compared to Vehicle and Olanzapine by separate Dunnett’s tests and to Olanzapine/Vehicle by Williams’ test. Other comparisons by multiple t test. *p<0.05, **p<0.01, ***p<0.001.

| Treatment | n | Mean | SEM | vs Vehicle (A) | | | vs Vehicle (F) | | vs Olanzapine (B) | |
|---|---|---|---|---|---|---|---|---|---|---|
| | | | | Difference | % change | p | Difference | p | Difference | p |
| Fat per rat (g) | | | | | | | | | | |
| Vehicle (aqueous methyl cellulose) po | 12 | 35.3 | 1.6 | | | | | | | |
| Olanzapine - 3 mg/kgpo | 12 | 43.3 | 1.7 | 8.0 | 22.7 | 0.007** | | | | |
| Compound 1 - 0.3 mg/kg po | 12 | 32.7 | 2.1 | -2.6 | -7.4 | 0.698 | -3.7 | 0.201 | -10.6 | 0.001** |
| Compound 1 -- 1 mg/kg po | 12 | 32.3 | 1.6 | -2.9 | -8.3 | 0.623 | -4.0 | 0.195 | -10.9 | 0.001** |
| Compound1 - 3 mg/kg po | 12 | 32.0 | 1.9 | -3.3 | -9.4 | 0.535 | -4.4 | 0.161 | -11.3 | <0.001*** |
| Vehicle (water) po | 13 | 36.3 | 1.9 | 1.1 | 3.1 | 0.706 | | | -6.9 | 0.018* |
| Protein in carcass (%) | | | | | | | | | | |
| Vehicle (aqueous methyl cellulose) po | 12 | 20.73 | 0.20 | | | | | | | |
| Olanzapine - 3 mg/kg po | 12 | 20.24 | 0.17 | -0.49 | -2.4 | 0.150 | | | | |
| Compound 1 - 0.3 mg/kg po | 12 | 20.90 | 0.23 | 0.17 | 0.8 | 0.924 | 0.36 | 0.273 | 0.66 | 0.135 |
| Compound 1 - 1 mg/kg po | 12 | 21.00 | 0.17 | 0.27 | 1.3 | 0.756 | 0.47 | 0.189 | 0.76 | 0.069 |
| Compound 1 -- 3 mg/kg po | 12 | 21.18 | 0.22 | 0.45 | 2.2 | 0.403 | 0.65 | 0.065 | 0.94 | 0.019* |
| Vehicle (water) po | 13 | 20.53 | 0.20 | -0.20 | -0.9 | 0.554 | | | 0.29 | 0.377 |

Protein per rat (g)

| | | | | | | | | | | |
|---|---|---|---|---|---|---|---|---|---|---|
| Vehicle (aqueous methyl cellulose) po | 12 | 54.1 | 0.6 | | | | | | | |
| Olanzapine - 3 mg/kg po | 12 | 55.0 | 0.4 | 0.9 | 1.6 | 0.321 | | | | |
| Compound 1 -- 0.3 mg/kg po | 12 | 54.7 | 0.7 | 0.6 | 1.1 | 0.841 | -0.4 | 0.609 | -0.3 | 0.976 |
| Compound 1 - 1 mg/kg po | 12 | 53.7 | 0.5 | -0.4 | -0.8 | 0.921 | -1.5 | 0.117 | -1.3 | 0.309 |
| Compound 1 - 3 mg/kg po | 12 | 53.7 | 0.7 | -0.4 | -0.7 | 0.938 | -1.4 | 0.117 | -1.3 | 0.333 |
| Vehicle (water) po | 13 | 55.1 | 0.6 | 1.0 | 1.9 | 0.235 | | | 0.2 | 0.858 |

Final carcass weight (g)

| | | | | | | | | | | |
|---|---|---|---|---|---|---|---|---|---|---|
| Vehicle (aqueous methyl cellulose) po | 12 | 261.3 | 1.3 | | | | | | | |
| Olanzapine - 3 mg/kg po | 12 | 271.9 | 2.3 | 10.6 | 4.1 | 0.006** | | | | |
| Compound 1 - 0.3 mg/kg po | 12 | 261.4 | 4.1 | 0.1 | 0.0 | >0.999 | -6.7 | 0.073 | -10.5 | 0.017* |
| Compound 1 - 1 mg/kg po | 12 | 255.4 | 2.0 | -5.9 | -2.2 | 0.280 | -12.7 | 0.001** | -16.5 | <0.001*** |
| Compound 1 - 3 mg/kg po | 12 | 255.3 | 2.1 | -6.0 | -2.3 | 0.264 | -12.8 | <0.001*** | -16.6 | <0.001*** |
| Vehicle (water) po | 13 | 268.1 | 2.8 | 6.8 | 2.6 | 0.068 | | | -3.8 | 0.198 |

Means are adjusted for Day 1 body weight differences between the treatment groups. Standard errors of the mean (SEM) are calculated from the residuals of the statistical model. Compound 1 - was compared to Vehicle and Olanzapine by separate Dunnett’s tests and to Olanzapine/Vehicle by Williams’ test. Other comparisons by multiple t test. *p<0.05, **p<0.01, ***p<0.001.

TABLE 2.2.4

Liver triglycerides data

| Treatment | n | Mean | SEM | vs Vehicle (A) | | vs Vehicle (F) | | vs Olanzapine (B) | |
|---|---|---|---|---|---|---|---|---|---|
| | | | | % | p | % | p | % | p |
| Triglycerides (µmol/g) | | | | | | | | | |
| Vehicle (aqueous methyl cellulose) po | 12 | 37.1 | 8.2 | | | | | | |
| Olanzapine - 3 mg/kg po | 12 | 37.1 | 8.2 | 100.0 | >0.999 | | | | |
| Compound 1 -0.3 mg/kg po | 12 | 26.5 | 6.8 | 71.3 | 0.569 | 84.0 | 0.572 | 71.3 | 0.569 |
| Compound 1 - 1 mg/ka po | 12 | 20.3 | 3.8 | 54.7 | 0.143 | 64.5 | 0.538 | 54.7 | 0.143 |
| Compound 1 - 3 mg/kg po | 12 | 29.7 | 6.9 | 80.0 | 0.816 | 94.3 | 0.538 | 80.0 | 0.816 |
| Vehicle (water) po | 13 | 31.5 | 5.4 | 84.8 | 0.593 | | | 84.8 | 0.593 |

Triglycerides (µmol total)

| | | | | | | | | | |
|---|---|---|---|---|---|---|---|---|---|
| Vehicle (aqueous methyl cellulose) | 12 | 346 | 76 | | | | | | |
| po | | | | | | | | | |
| Olanzapine - 3 mg/kg po | 12 | 348 | 79 | 100.4 | 0.989 | | | | |
| Compound 1 - 0.3 mg/kg po | 12 | 242 | 64 | 70.0 | 0.538 | 82.8 | 0.547 | 69.7 | 0.529 |
| Compound 1 - 1 mg/kg po | 12 | 175 | 34 | 50.5 | 0.088 | 59.8 | 0.404 | 50.3 | 0.086 |
| Compound 1 - 3 mg/kg po | 12 | 261 | 59 | 75.4 | 0.702 | 89.3 | 0.404 | 75.1 | 0.693 |
| Vehicle (water) po | 13 | 293 | 52 | 84.5 | 0.588 | | | 84.1 | 0.579 |

Means are back transformed and adjusted for termination order and body weight differences between the treatment groups on Day 1. Standard errors of the mean (SEM) are calculated from the residuals of the statistical model. Compound 1 - was compared to Vehicle (A) and Olanzapine (B) by separate Dunnett’s tests and Vehicle (F) by Williams test. Other comparisons by the multiple t test.

### Example 2.3: Effect of Compound 1 on Olanzapine-Induced Weight Gain in Female Sprague Dawley Rats

Compound 1 is being investigated to explore the effects of administration of Compound 1 once daily for a maximum of 15 days in female Sprague Dawley rats fed a high-fat powdered diet. The effect of the compound is assessed both alone and in the presence of olanzapine. Sibutramine is included as a reference comparator. This model is sensitive to the effects of olanzapine on body weight. The aim of the study is to investigate whether administration of the test compound affects olanzapine-induced weight gain and whether, at the doses tested, the compound has any effect on body weight and food and water intake when administered alone.

### Materials and Methods

Animals: Seventy-four (2 spares) female Sprague Dawley rats (weight range 200-300 g) are ordered from Charles River, Margate, Kent. Rats are singly housed in cages with sawdust bedding and enrichment at an ambient temperature of 21 ± 2° C. Upon arrival (Thursday), rats are weighed and given wet mash to aid recovery from transport. Rats are weighed the following day and the Monday of the following week. Animals are maintained on a reverse-phase light-dark cycle (lights off for 8 h from 10:00-18 :00 h) during which time the room will be illuminated by red light. Rats are weighed on the Friday and Monday of the following week. Relative humidity is typically 55 ± 15% with prolonged periods below 40%RH or above 70%RH avoided as detailed in the UK Code of Practice. All animals have free access to a high-fat powdered diet (VRF1 plus 20% lard) and all rats have access to filtered water at all times. The diets are contained in glass feeding jars with aluminum lids (Solmedia Laboratory Suppliers, Romford, Essex). Each lid has a 3-4 cm hole cut in it to allow access to the food. Animals are accustomed to these conditions for at least two weeks before experimentation. Sibutramine is used as a reference agent.

Experimental Procedures: Animals undergo a 3-day baseline run-in period during which time all rats are dosed once a day with vehicle. Dosing is timed to start at approximately 08:45 so that the mid-point is approximately the time of lights out. Animals, food and water are weighed at the time of dosing. Towards the end of the baseline period, animals are weighed (to the nearest 0.1 g using an electronic top-pan balance) and allocated by a statistician (see table below). The spare animals are humanely withdrawn from the study. Subsequently, rats are dosed once daily as follows: Treatment 1: Group A - Vehicle (5 mL/kg po) (n = 12); Group B - Vehicle (5 mg/kg po) (n = 12); Group C - Compound 1 (po) (n = 12); Group D - Compound 1 (po) (n = 12); Group E - Sibutramine (po) (n = 12); Group F - Sibutramine (po) (n = 12); Treatment 2: Group A - Vehicle (5 mL/kg po) (n = 12); Group B - Olanzapine (3 mg/kg po) (n = 12); Group C - Vehicle (3 mg/kg po) (n = 12); Group D - Olanzapine (3 mg/kg po) (n = 12); Group E -Vehicle (5 mL/kg po) (n = 12); Group F - Olanzapine (3 mg/kg) (n = 12).

Dosing begins at approximately 08 :45 h each day (0h), i.e., the mid-point of dosing is approximately at the time of lights out. Treatment 2 is administered as soon as possible after Treatment 1. Body weight, food and water intake are recorded daily at the time of dosing. All rats are observed before and after dosing and comments on condition are noted as appropriate.

On the afternoon of Day 14 (approx. 16:00) animals are fasted to a timed schedule. The experiment ends the following morning on Day 15. On the morning of Day 15, final readings are undertaken 16 h post-fast (i.e., body weight measurements). Animals are then dosed (as described). Four hours later, a blood sample (1 mL) is taken from the lateral tail vein into tubes containing lithium heparin. The blood is centrifuged at 2,400 g for 5 min at 4° C. to produce five aliquots of plasma which are stored frozen for optional plasma analysis at Sygnature Discovery Ltd (glucose, insulin, triglycerides, cholesterol (HDL, LDL, total) and NEFA). Animals are then terminated by a Schedule 1 method (exposure to an increasing concentration of CO₂ with death confirmed by cervical dislocation). A terminal blood sample (circa 4 mL) is taken into a lithium heparin-coated tube and a single plasma aliquot (approximately 1 mL) is stored frozen (approx. -80° C.) in a clean aliquot tube. Carcasses are stored frozen (approx. -20° C.) for potential body composition analysis.

### Example 3: Diet-Induced Obesity (DIO) Mouse Model

### Effect of Compound 1 in the Diet-Induced Obese (DIO) Mouse Model

The diet induced obese (DIO) mouse is a well characterized model of obesity which exhibits increased adiposity, insulin resistance and glucose intolerance (Kleinert et al., 2018). The aim of this study was to determine the effect of Compound 1, administered orally once daily for a 35-day period, in male mice exhibiting obesity due to long term access to a high-fat diet (45% kcal derived from fat). The GLP1 receptor agonist, liraglutide, was used as a positive reference control. The effect of once-daily oral administration of Compound 1 was assessed daily on body weight, food and water intake for 35 days. In addition, fasting plasma glucose and insulin were measured on day 15 and an oral glucose tolerance test (OGTT) was conducted on day 28. Body composition was analyzed by DEXA on Day 25.

### Materials and Methods

Animals: Seventy-five (72 main study; 3 spares) male C57B1/6J mice were ordered from Charles River UK (Margate, Kent UK) at 7-8 weeks of age. Upon arrival, animals were group housed on a normal light/dark cycle (lights on: 07:00-19 :00h) with ad libitum access to a high-fat diet (D12451 diet, 45% kcals as fat; Research Diets, New Jersey, USA) and filtered tap water. After exposure to the high fat diet for at least 14 weeks, animals were singly housed on a reverse phase light/dark cycle (lights off for 8 hours from 9:30-17:30 h) and were provided with high fat diet (D12451; 20% protein, 35% carbohydrate and 45% fat; Research Diets, New Jersey, USA) and filtered water ad libitum for the study duration. Animals were weighed weekly until the start of the baseline phase; thereafter they were weighed daily.

Experimental Procedures: The handling procedure reduced the incidence of stress-related effects in studies (e.g., in a chronic study the body weight response to baseline dosing is more stable). This handling/habituation phase continued once daily for 7 days (avoiding weekends). Subsequent to the handling phase, animals underwent a 7-day baseline phase where they were dosed once daily with vehicle via the oral and subcutaneous route. All dosing began at approximately 08:30 am each day such that the mid-point of morning doing was approximately 09:30 am. During the baseline and drug treatment phases, body weight, food and water intake were recorded daily at the time of dosing. All mice were observed before and after dosing and comments on condition were noted as appropriate. The mice were allocated to 6 groups on the basis of body weight, food and water intake by a statistician as follows: Group A - Vehicle (deionized water po) (n = 12); Group B - Compound 1 (0.3 mg/kg po) (n = 12); Group C -Compound 1 (1 mg/kg po) (n = 12); Group D - Compound 1 (3 mg/kg po) (n = 12); Group E -Compound 1 (10 mg/kg po) (n = 12); Group F - Liraglutide (0.2 mg/kg subcutaneous) (n = 12). The spare animals were humanely withdrawn from the study or included in a group at the discretion of a statistician.

On Day 1 onward, all mice were dosed once daily with vehicle, Compound 1, or liraglutide. All dosing began at approximately 08:30 each day and is via the oral or subcutaneous (sc) route with a mid-point of circa 09:30. Body weight, food and water intake were recorded daily at the time of morning dosing. All mice were observed before and after each dosing session and comments on condition were noted as appropriate. For the subcutaneous injection the site was rotated daily to minimize potential irritation at the site of needle entry. Dosing continued until a blood sample is take on Day 15.

Day 15 Blood Sampling (glucose and insulin): On Day 15 animals had food removed for 4 hours to a timed schedule. Three hours post fast, each mouse was dosed (again to a timed schedule). Thirty minutes later a single blood sample (30 µl) was taken into lithium heparin-coated tubes (Sarstedt CB300LH) from the lateral tail vein and spun as soon as possible in a centrifuge. Plasma was dispensed into a clean aliquot tube and frozen immediately on dry ice. Plasma samples were stored frozen (approx. -80° C.) until determination of plasma glucose (Thermoelectron Infinity glucose reagent TR15421) and insulin (Alpco mouse ultrasensitive insulin kit 80 INSMSU-E10). Dosing continued until a DEXA is undertaken on Day 25.

Day 25 DEXA: On Day 25, body composition analysis was performed by DEXA on mice anaesthetized with isoflurane using the Lunar PIXImus Densitometer (readings were taken post dose but not to a timed schedule). Analysis of the mice was with a defined region of interest (ROI) to exclude the head. Thus, results were generated with and without the inclusion of the head. Results for sample weight (g), lean mass (g), fat mass (g), fat mass (%), BMC (bone mineral content), and BMD (bone mineral density) were reported. Mice were dosed and body weight, food and water intake are measured as on all previous days. DEXA scanning was not performed to a timed schedule relative to dosing. The study continued until performance of an oral glucose test (OGTT).

Day 29 OGTT: On Day 28, mice were fasted (food will be weighed) beginning at approximately 16:00 to a timed schedule. An OGTT was performed after an overnight fast on the following morning (approx. 16 hours post fast). The OGTT was undertaken in a room on a normal light/dark cycle. The animals were moved to a separate room which was maintained under normal lighting. Mice were dosed to a timed schedule 30 minutes prior to the administration of the glucose challenge (2.0 g/kg po). Blood samples were taken immediately prior to dosing (B1, 30 µl), immediately prior to glucose administration (B2, 30 µl) and 15 minutes (30 µl), 30 minutes (30 µl), 60 minutes (30 µl) and 120 minutes (30 µl) after glucose administration. All blood samples were taken into lithium heparin-coated tubes (Sarstedt CB300LH) and spun as soon as possible in a centrifuge. Plasma samples were stored frozen (approx. -80° C.) until determination of plasma glucose (Thermoelectron Infinity glucose reagent TR15421) and insulin (Alpco mouse ultrasensitive insulin kit 80 INSMSU-E10). At the end of the OGTT, mice have a weighed amount of food returned and they were returned to their normal procedure room. Dosing continues until Day 35.

Day 35 Termination: On Day 35 all mice were dosed and 30 minutes after dosing mice were humanely killed by a schedule 1 method (exposure to an increasing concentration of CO₂ with cervical dislocation). A post-mortem blood sample (approximately 0.6 mL) was taken by cardiac puncture. Two blood samples were collected: one of 200 µl in an EDTA-coated tube (for bioanalysis) and another in a lithium heparin-coated tube. The plasma samples were stored (approx. -80° C.) until sent for bioanalysis or assayed for triglycerides, NEFA and cholesterol. Immediately after blood sampling, the liver was removed and weighed. The right lobe was dissected and one portion (80-129 mg, weighed) was stored in a cryovial (3.6 mL) and the remaining right lobe stored in a screwcap microtube (CP5915, Alpha laboratories). Samples were frozen immediately on dry ice prior to storage at approximately -80° C. These samples may be used for the analysis of liver triglycerides (TRIGL: Cat. 04657594 190), cholesterol and NEFA.

Results: Single daily administration of Compound 1 (0.3, 1, 3 and 10 mg/kg, po) for 35 days dose-dependently reduced body weight in DIO mice (Table 3.1), with the largest decrease of 7.5% at the 10 mg/kg dose. Weight loss occurred without significant effect on food intake (Table 3.2), while there was a dose-dependent increase in water intake (Table 3.3), potentially suggesting positive effects on energy expenditure. Body composition analysis was performed by DEXA on Day 25 (Table 3.4). Total fat mass determined was slightly reduced at the 10 mg/kg dose, consistent with the modest weight loss observed on that day. On day 15, following a 4 hr fast, animals were dosed, and plasma samples collected 30 min later. Compound 1 dose-dependently reduced elevated fasting glucose and insulin levels in DIO mice suggesting improved insulin sensitivity (Table 3.5). Plasma lowering of glucose and insulin was also observed in the oGTT conducted on Day 28, following a 16 hr fast (Tables 3.6 - 3.11). This is consistent with previous results in naive and diabetic db/db mice and reflects improved insulin sensitivity and/or effects on gastric emptying following Compound 1 treatment. Compound 1 also dose-dependently decreased liver weights with a trend observed for a reduction in liver triglycerides (Tables 3.12 and 3.13). Plasma lipid parameters were not significantly changed by Compound 1. Overall, Compound 1 reduced body weight and improved glycemic control in the DIO mouse model.

TABLE 3.1

Changes in body weights (g)

| Time | Treatment | n | Mean | SEM | Difference | p |
|---|---|---|---|---|---|---|
| Weeks 1-5 | Vehicle (de-ionised water) po | 12 | -0.29 | 0.49 | | |
| (Day 1-35) | Compound 10.3 mg/kg po | 12 | -1.48 | 0.32 | -1.19 | 0.092 |
| | Compound 1 - 1 mg/kg po | 12 | -1.83 | 0.51 | -1.53 | 0.035* |
| | Compound 1 - 3 mg/kg po | 12 | -2.69 | 0.43 | -2.40 | 0.001** |
| | Compound 1 - 10 mg/kg po | 12 | -3.46 | 0.55 | -3.17 | <0.001** * |
| | Liraglutide 0.2 mg/kg sc | 11 | -8.00 | 0.62 | -7.71 | <0.001** * |

Means are adjusted for differences between the treatment groups at baseline (Day 1) Standard errors of the mean (SEM) are calculated from the residuals of the statistical model Comparisons to vehicle were by Williams’ test for Compound 1 - and the multiple t test for Liraglutide. *p<0.05, **p<0.01, ***p<0.001

TABLE 3.2

Averages of daily food intake (g/day)

| Time | Treatment | n | Mean | SEM | Difference | p |
|---|---|---|---|---|---|---|
| Weeks 1-5 | Vehicle (de-ionised water) po | 12 | 2.69 | 0.05 | | |
| (Day 1-34) | Compound 10.3 mg/kg po | 12 | 2.55 | 0.05 | -0.14 | 0.426 |
| | Compound 1 - 1 mg/kg po | 12 | 2.66 | 0.05 | -0.03 | 0.426 |
| | Compound 1 - 3 mg/kg po | 12 | 2.65 | 0.05 | -0.03 | 0.426 |
| | Compound 1 - 10 mg/kg po | 12 | 2.59 | 0.04 | -0.09 | 0.226 |
| | Liraglutide 0.2 mg/kg sc | 11 | 2.21 | 0.06 | -0.47 | <0.001*** |

Means are adjusted for differences between the treatment groups at baseline (average of Days -6 to 0) Standard errors of the mean (SEM) are calculated from the residuals of the statistical model Comparisons to vehicle were by Williams’ test for Compound 1 - and the multiple t test for Liraglutide. *p<0.05, **p<0.01, ***p<0.001

TABLE 3.3

Averages of daily water intake (g/day)

| Time | Treatment | n | Mean | SEM | Difference | p |
|---|---|---|---|---|---|---|
| Weeks 1-5 | Vehicle (de-ionised water) po | 12 | 3.21 | 0.08 | | |
| (Day 1-34) | Compound 10.3 mg/kg po | 12 | 3.49 | 0.11 | 0.28 | 0.065 |
| | Compound 1 - 1 mg/kg po | 12 | 3.66 | 0.10 | 0.45 | 0.004** |
| | Compound 1 - 3 mg/kg po | 12 | 3.68 | 0.12 | 0.46 | 0.003** |
| | Compound 1 - 10 mg/kg po | 12 | 3.86 | 0.08 | 0.65 | <0.001*** |
| | Liraglutide 0.2 mg/kg sc | 11 | 3.27 | 0.12 | 0.05 | 0.724 |

Means are adjusted for differences between the treatment groups at baseline (average of Days -6 to 0) Standard errors of the mean (SEM) are calculated from the residuals of the statistical model Comparisons to vehicle were by Williams’ test for Compound 1 - and the multiple t test for Liraglutide. *p<0.05, **p<0.01, ***p<0.00

TABLE 3.4

DEXA (Day 25)

| Treatment | n | Mean | SEM | Difference | p |
|---|---|---|---|---|---|
| TTM (g) | | | | | |
| Vehicle (de-ionised water) po | 12 | 41.56 | 0.37 | | |
| Compound 1 - 0.3 mg/kg po | 12 | 41.12 | 0.37 | -0.43 | 0.925 |
| Compound 1 - 1 mg/kg po | 12 | 41.61 | 0.39 | 0.05 | 0.925 |
| Compound 1 - 3 mg/kg po | 11 | 40.53 | 0.48 | -1.03 | 0.188 |
| Compound 1 - 10 mg/kg po | 12 | 39.40 | 0.49 | -2.16 | 0.003** |
| Liraglutide 0.2 mg/kg sc | 11 | 34.51 | 0.77 | -7.05 | <0.001*** |
| Fat mass (g) | | | | | |
| Vehicle (de-ionised water) po | 12 | 15.78 | 0.52 | | |
| Compound 1 - 0.3 mg/kg po | 12 | 16.26 | 0.41 | 0.48 | >0.999 |
| Compound 1 - 1 mg/kg po | 12 | 15.63 | 0.48 | -0.15 | >0.999 |
| Compound 1 - 3 mg/kg po | 11 | 15.53 | 0.59 | -0.25 | 0.956 |
| Compound 1 - 10 mg/kg po | 12 | 13.92 | 0.63 | -1.86 | 0.028* |
| Liraglutide 0.2 mg/kg sc | 11 | 9.75 | 0.73 | -6.03 | <0.001*** |

Fat mass (%)

| | | | | | |
|---|---|---|---|---|---|
| Vehicle (de-ionised water) po | 12 | 37.45 | 1.20 | | |
| Compound 1 - 0.3 mg/kg po | 12 | 38.93 | 1.06 | 1.48 | >0.999 |
| Compound 1 - 1 mg/kg po | 12 | 37.32 | 1.03 | -0.13 | >0.999 |
| Compound 1 - 3 mg/kg po | 11 | 37.83 | 1.31 | 0.38 | >0.999 |
| Compound 1 - 10 mg/kg po | 12 | 35.08 | 1.29 | -2.37 | 0.260 |
| Liraglutide 0.2 mg/kg sc | 11 | 27.32 | 1.81 | -10.13 | <0.001*** |

Lean mass (g)

| | | | | | |
|---|---|---|---|---|---|
| Vehicle (de-ionised water) po | 12 | 25.78 | 0.41 | | |
| Compound 1 - 0.3 mg/kg po | 12 | 24.87 | 0.52 | -0.91 | 0.662 |
| Compound 1 - 1 mg/kg po | 12 | 25.98 | 0.42 | 0.20 | 0.662 |
| Compound 1 - 3 mg/kg po | 11 | 25.00 | 0.38 | -0.78 | 0.502 |
| Compound 1 - 10 mg/kg po | 12 | 25.48 | 0.38 | -0.30 | 0.502 |
| Liraglutide 0.2 mg/kg sc | 11 | 24.76 | 0.38 | -1.02 | 0.101 |
| Vehicle (de-ionised water) po | 12 | 62.55 | 1.20 | | |
| Compound 1 - 0.3 mg/kg po | 12 | 61.07 | 1.06 | -1.48 | >0.999 |
| Compound 1 - 1 mg/kg po | 12 | 62.68 | 1.03 | 0.13 | >0.999 |
| Compound 1 - 3 mg/kg po | 11 | 62.17 | 1.31 | -0.38 | >0.999 |
| Compound 1 - 10 mg/kg po | 12 | 64.92 | 1.29 | 2.37 | 0.260 |
| Liraglutide 0.2 mg/kg sc | 11 | 72.68 | 1.81 | 10.13 | <0.001*** |

Means are adjusted for differences between the treatment groups in body weight on Day 1 and scan order. TTM=Total mass. Standard errors of the mean (SEM) are calculated from the residuals of the statistical model. Comparisons to vehicle were by Williams’ test for Compound 1 - and the multiple t test for Liraglutide. *p<0.05, **p<0.01, ***p<0.001

TABLE 3.5

Day 15 Plasma glucose (mM) and insulin (ng/mL)

| Treatment | n | Mean | SEM | Difference | p |
|---|---|---|---|---|---|
| Plasma glucose (mM) | | | | | |
| Vehicle (de-ionised water) po | 12 | 13.98 | 0.32 | | |
| Compound 1 - 0.3 mg/kg po | 12 | 13.31 | 0.20 | -0.68 | 0.186 |
| Compound 1 - 1 mg/kg po | 12 | 12.68 | 0.33 | -1.30 | 0.014* |
| Compound 1 - 3 mg/kg po | 12 | 12.56 | 0.31 | -1.42 | 0.007** |
| Compound 1 - 10 mg/kg po | 12 | 11.74 | 0.47 | -2.24 | <0.001*** |
| Liraglutide 0.2 mg/kg sc | 11 | 11.59 | 0.59 | -2.39 | <0.001*** |

Plasma insulin (ng/mL)

| | | | | | |
|---|---|---|---|---|---|
| Vehicle (de-ionised water) po | 12 | 2.26 | 0.18 | | |
| Compound 1 - 0.3 mg/kg po | 12 | 1.82 | 0.12 | -0.45 | 0.113 |
| Compound 1 - 1 mg/kg po | 12 | 1.85 | 0.18 | -0.42 | 0.113 |
| Compound 1 - 3 mg/kg po | 12 | 1.70 | 0.24 | -0.56 | 0.038* |
| Compound 1 - 10 mg/kg po | 12 | 1.37 | 0.21 | -0.89 | <0.001*** |
| Liraglutide 0.2 mg/kg sc | 11 | 0.57 | 0.11 | -1.69 | <0.001*** |

Means are adjusted for bleeding order and differences between the treatment groups in body weight on Day 1. Standard errors of the mean (SEM) are calculated from the residuals of the statistical model. Analysis was by robust regression of untransformed data. Comparisons to vehicle were by Williams’ test for Compound 1 - and the multiple t test for Liraglutide. *p<0.05, **p<0.01, ***p<0.001

TABLE 3.6

OGTT Plasma glucose (mM)

| Treatment | n | Mean | SEM | % of vehicle | p |
|---|---|---|---|---|---|
| Pre-treatment baseline (B1) | | | | | |
| Vehicle (de-ionised water) po | 12 | 14.64 | 0.49 | | |
| Compound 10.3 mg/kg po | 12 | 14.21 | 0.65 | -0.43 | 0.921 |
| Compound 1 - 1 mg/kg po | 12 | 14.73 | 0.54 | 0.10 | 0.921 |
| Compound 1 - 3 mg/kg po | 12 | 14.25 | 0.75 | -0.39 | 0.647 |
| Compound 1 - 10 mg/kg po | 12 | 13.68 | 0.39 | -0.96 | 0.130 |
| Liraglutide 0.2 mg/kg sc | 11 | 10.56 | 0.32 | -4.08 | <0.001*** |
| Post-treatment baseline (B2) | | | | | |
| Vehicle (de-ionised water) po | 12 | 17.55 | 0.63 | | |
| Compound 10.3 mg/kg po | 12 | 15.67 | 0.90 | -1.89 | 0.048* |
| Compound 1 - 1 mg/kg po | 12 | 14.95 | 0.73 | -2.60 | 0.027* |
| Compound 1 - 3 mg/kg po | 12 | 15.53 | 1.04 | -2.02 | 0.027* |
| Compound 1 - 10 mg/kg po | 12 | 15.62 | 0.54 | -1.93 | 0.027* |
| Liraglutide 0.2 mg/kg sc | 11 | 11.72 | 0.55 | -5.84 | <0.001*** |
| 15 minutes | | | | | |
| Vehicle (de-ionised water) po | 11 | 28.25 | 0.63 | | |
| Compound 10.3 mg/kg po | 12 | 21.53 | 1.48 | -6.72 | <0.001*** |
| Compound 1 - 1 mg/kg po | 12 | 17.27 | 0.58 | -10.98 | <0.001*** |
| Compound 1 - 3 mg/kg po | 12 | 17.42 | 0.57 | -10.83 | <0.001*** |
| Compound 1 - 10 mg/kg po | 12 | 17.60 | 0.43 | -10.65 | <0.001*** |
| Liraglutide 0.2 mg/kg sc | 11 | 25.43 | 0.40 | -2.82 | 0.004** |
| 30 minutes | | | | | |
| Vehicle (de-ionised water) po | 11 | 23.33 | 0.86 | | |
| Compound 1 - 0.3 mg/kg po | 12 | 24.60 | 1.67 | 1.27 | >0.999 |
| Compound 1 - 1 mg/kg po | 11 | 21.61 | 1.32 | -1.72 | 0.297 |
| Compound 1 - 3 mg/kg po | 12 | 20.23 | 1.19 | -3.11 | 0.251 |
| Compound 1 - 10 mg/kg po | 12 | 22.63 | 0.69 | -0.71 | 0.251 |
| Liraglutide 0.2 mg/kg sc | 11 | 19.87 | 0.71 | -3.46 | 0.023* |
| 60 minutes | | | | | |
| Vehicle (de-ionised water) po | 11 | 22.53 | 1.35 | | |
| Compound 10.3 mg/kg po | 12 | 24.57 | 1.41 | 2.03 | 0.777 |
| Compound 1 - 1 mg/kg po | 12 | 23.11 | 1.49 | 0.58 | 0.777 |
| Compound 1 - 3 mg/kg po | 12 | 22.66 | 1.54 | 0.13 | 0.777 |
| Compound 1 - 10 mg/kg po | 12 | 25.69 | 1.07 | 3.16 | 0.103 |
| Liraglutide 0.2 mg/kg sc | 11 | 14.23 | 0.82 | -8.30 | <0.001*** |

120 minutes

| | | | | | |
|---|---|---|---|---|---|
| Vehicle (de-ionised water) po | 11 | 17.86 | 1.09 | | |
| Compound 10.3 mg/kg po | 12 | 18.99 | 1.11 | 1.13 | 0.374 |
| Compound 1 - 1 mg/kg po | 12 | 21.23 | 1.14 | 3.36 | 0.036* |
| Compound 1 - 3 mg/kg po | 12 | 20.06 | 1.24 | 2.20 | 0.036* |
| Compound 1 - 10 mg/kg po | 12 | 21.98 | 0.82 | 4.12 | 0.002** |
| Liraglutide 0.2 mg/kg sc | 11 | 11.67 | 0.76 | -6.19 | <0.001*** |

Means are adjusted for bleeding order and differences between the treatment groups in body weight on Day 1. Standard errors of the mean (SEM) are calculated from the residuals of the statistical model. Analysis was by robust regression of untransformed data. Comparisons to vehicle were by Williams’ test for Compound 1 - and the multiple t test for Liraglutide. *p<0.05, **p<0.01, ***p<0.001

TABLE 3.7

OGTT Plasma glucose (mM) AUC

| Treatment | n | Mean | SEM | Difference | p |
|---|---|---|---|---|---|
| Area under curve (0-60 mins) | | | | | |
| Vehicle (de-ionised water) po | 11 | 23.72 | 0.65 | | |
| Compound 1 - 0.3 mg/kg po | 12 | 22.91 | 1.29 | -0.81 | 0.432 |
| Compound 1 - 1 mg/kg po | 11 | 19.89 | 0.94 | -3.83 | 0.001** |
| Compound 1 - 3 mg/kg po | 12 | 19.37 | 0.85 | -4.35 | <0.001*** |
| Compound 1 - 10 mg/kg po | 12 | 21.15 | 0.49 | -2.57 | <0.001*** |
| Liraglutide 0.2 mg/kg sc | 11 | 18.85 | 0.44 | -4.87 | <0.001*** |

Area under curve (0-120 mins)

| | | | | | |
|---|---|---|---|---|---|
| Vehicle (de-ionised water) po | 11 | 43.98 | 1.77 | | |
| Compound 1 - 0.3 mg/kg po | 12 | 43.95 | 2.32 | -0.02 | 0.992 |
| Compound 1 - 1 mg/kg po | 11 | 42.24 | 2.16 | -1.73 | 0.715 |
| Compound 1 - 3 mg/kg po | 12 | 40.23 | 1.92 | -3.75 | 0.715 |
| Compound 1 - 10 mg/kg po | 12 | 45.19 | 1.09 | 1.21 | 0.715 |
| Liraglutide 0.2 mg/kg sc | 11 | 31.69 | 0.86 | -12.29 | <0.001*** |

Means are adjusted for bleeding order and differences between the treatment groups in body weight on Day 1. Standard errors of the mean (SEM) are calculated from the residuals of the statistical model. Analysis was by robust regression of untransformed data. Comparisons to vehicle were by Williams’ test for Compound 1 - and the multiple t test for Liraglutide. **p<0.01, ***p<0.001

TABLE 3.8

OGTT Plasma glucose (mM) AUC above baseline

| Treatment | n | Mean | SEM | Difference | p |
|---|---|---|---|---|---|
| AUC above baseline (0-60 mins) | | | | | |
| Vehicle (de-ionised water) po | 11 | 6.40 | 0.55 | | |
| Compound 1 - 0.3 mg/kg po | 12 | 6.85 | 0.77 | 0.45 | >0.999 |
| Compound 1 - 1 mg/kg po | 11 | 4.69 | 0.60 | -1.71 | 0.199 |
| Compound 1 - 3 mg/kg po | 12 | 3.95 | 1.48 | -2.45 | 0.199 |
| Compound 1 - 10 mg/kg po | 12 | 5.51 | 0.73 | -0.89 | 0.199 |
| Liraglutide 0.2 mg/kg sc | 11 | 7.15 | 0.57 | 0.75 | 0.529 |

AUC above baseline (0-120 mins)

| | | | | | |
|---|---|---|---|---|---|
| Vehicle (de-ionised water) po | 11 | 9.54 | 1.25 | | |
| Compound 1 - 0.3 mg/kg po | 12 | 12.61 | 1.43 | 3.07 | 0.691 |
| Compound 1 - 1 mg/kg po | 11 | 11.09 | 1.64 | 1.56 | 0.691 |
| Compound 1 - 3 mg/kg po | 12 | 9.46 | 3.26 | -0.07 | 0.691 |
| Compound 1 - 10 mg/kg po | 12 | 13.98 | 1.60 | 4.45 | 0.095 |
| Liraglutide 0.2 mg/kg sc | 11 | 8.25 | 1.00 | -1.29 | 0.612 |

Means are adjusted for bleeding order and differences between the treatment groups in body weight on Day 1. Standard errors of the mean (SEM) are calculated from the residuals of the statistical model. Analysis was by robust regression of untransformed data. Comparisons to vehicle were by Williams’ test for Compound 1 - and the multiple t test for Liraglutide.

TABLE 3.9

OGTT Plasma insulin (ng/mL)

| Treatment | n | Mean | SEM | Difference | p |
|---|---|---|---|---|---|
| Pre-treatment baseline (B1) | | | | | |
| Vehicle (de-ionised water) po | 12 | 1.46 | 0.10 | | |
| Compound 1 - 0.3 mg/kg po | 12 | 1.57 | 0.14 | 0.11 | >0.999 |
| Compound 1 - 1 mg/kg po | 12 | 1.59 | 0.20 | 0.13 | >0.999 |
| Compound 1 - 3 mg/kg po | 12 | 1.23 | 0.25 | -0.23 | 0.654 |
| Compound 1 - 10 mg/kg po | 12 | 1.39 | 0.18 | -0.07 | 0.654 |
| Liraglutide 0.2 mg/kg sc | 11 | 0.46 | 0.06 | -1.00 | <0.001*** |
| Post-treatment baseline (B2) | | | | | |
| Vehicle (de-ionised water) po | 12 | 2.02 | 0.11 | | |
| Compound 1 - 0.3 mg/kg po | 12 | 1.70 | 0.08 | -0.31 | 0.163 |
| Compound 1 - 1 mg/kg po | 12 | 1.74 | 0.16 | -0.28 | 0.163 |
| Compound 1 - 3 mg/kg po | 12 | 1.43 | 0.13 | -0.58 | 0.006** |
| Compound 1 - 10 mg/kg po | 12 | 1.47 | 0.14 | -0.55 | 0.006** |
| Liraglutide 0.2 mg/kg sc | 11 | 0.59 | 0.10 | -1.42 | <0.001*** |

15 minutes

| | | | | | |
|---|---|---|---|---|---|
| Vehicle (de-ionised water) po | 11 | 10.42 | 1.37 | | |
| Compound 1 - 0.3 mg/kg po | 12 | 6.21 | 0.57 | -4.21 | <0.001*** |
| Compound 1 - 1 mg/kg po | 12 | 3.85 | 0.60 | -6.58 | <0.001*** |
| Compound 1 - 3 mg/kg po | 12 | 3.36 | 0.29 | -7.07 | <0.001*** |
| Compound 1 - 10 mg/kg po | 12 | 3.42 | 0.40 | -7.00 | <0.001*** |
| Liraglutide 0.2 mg/kg sc | 11 | 4.89 | 0.84 | -5.53 | <0.001*** |
| Vehicle (de-ionised water) po | 11 | 5.27 | 0.47 | | |
| Compound 1 - 0.3 mg/kg po | 12 | 4.29 | 0.37 | -0.98 | 0.059 |
| Compound 1 - 1 mg/kg po | 12 | 3.10 | 0.60 | -2.17 | <0.001*** |
| Compound 1 - 3 mg/kg po | 12 | 2.57 | 0.27 | -2.70 | <0.001*** |
| Compound 1 - 10 mg/kg po | 12 | 3.10 | 0.31 | -2.17 | <0.001*** |
| Liraglutide 0.2 mg/kg sc | 11 | 2.70 | 0.26 | -2.57 | <0.001*** |
| Vehicle (de-ionised water) po | 11 | 2.31 | 0.18 | | |
| Compound 1 - 0.3 mg/kg po | 12 | 2.32 | 0.13 | 0.01 | >0.999 |
| Compound 1 - 1 mg/kg po | 12 | 2.46 | 0.26 | 0.15 | >0.999 |
| Compound 1 - 3 mg/kg po | 12 | 1.69 | 0.19 | -0.62 | 0.150 |
| Compound 1 - 10 mg/kg po | 12 | 2.14 | 0.19 | -0.18 | 0.150 |
| Liraglutide 0.2 mg/kg sc | 11 | 0.82 | 0.09 | -1.49 | <0.001*** |
| Vehicle (de-ionised water) po | 11 | 2.01 | 0.10 | | |
| Compound 1 - 0.3 mg/kg po | 12 | 1.92 | 0.13 | -0.09 | >0.999 |
| Compound 1 - 1 mg/kg po | 12 | 2.38 | 0.19 | 0.37 | 0.707 |
| Compound 1 - 3 mg/kg po | 12 | 1.86 | 0.12 | -0.15 | 0.707 |
| Compound 1 - 10 mg/kg po | 12 | 2.17 | 0.16 | 0.17 | 0.486 |
| Liraglutide 0.2 mg/kg sc | 11 | 0.55 | 0.07 | -1.46 | <0.001*** |

Means are adjusted for bleeding order and differences between the treatment groups in body weight on Day 1. Standard errors of the mean (SEM) are calculated from the residuals of the statistical model. Analysis was by robust regression of log transformed data. Comparisons to vehicle were by Williams’ test for Compound 1 - and the multiple t test for Liraglutide. **p<0.01, ***p<0.001

TABLE 3.10

OGTT Plasma insulin (ng/mL) AUC

| Treatment | n | Mean | SEM | Difference | p |
|---|---|---|---|---|---|
| Area under curve (0-60 mins) | | | | | |
| Vehicle (de-ionised water) po | 11 | 5.21 | 0.51 | | |
| Compound 1 - 0.3 mg/kg po | 12 | 4.07 | 0.19 | -1.15 | 0.008** |
| Compound 1 - 1 mg/kg po | 12 | 3.01 | 0.29 | -2.21 | <0.001*** |
| Compound 1 - 3 mg/kg po | 12 | 2.39 | 0.18 | -2.82 | <0.001*** |
| Compound 1 - 10 mg/kg po | 12 | 2.70 | 0.26 | -2.51 | <0.001*** |
| Liraglutide 0.2 mg/kg sc | 11 | 2.45 | 0.30 | -2.76 | <0.001*** |

Area under curve (0-120 mins)

| | | | | | |
|---|---|---|---|---|---|
| Vehicle (de-ionised water) po | 11 | 7.31 | 0.59 | | |
| Compound 1 - 0.3 mg/kg po | 12 | 6.16 | 0.23 | -1.14 | 0.042* |
| Compound 1 - 1 mg/kg po | 12 | 5.44 | 0.46 | -1.87 | 0.001** |
| Compound 1 - 3 mg/kg po | 12 | 4.19 | 0.31 | -3.11 | <0.001*** |
| Compound 1 - 10 mg/kg po | 12 | 4.85 | 0.41 | -2.46 | <0.001*** |
| Liraglutide 0.2 mg/kg sc | 11 | 3.16 | 0.34 | -4.15 | <0.001*** |

Means are adjusted for bleeding order and differences between the treatment groups in body weight on Day 1. Standard errors of the mean (SEM) are calculated from the residuals of the statistical model. Analysis was by robust regression of log transformed data. Comparisons to vehicle were by Williams’ test for Compound 1 - and the multiple t test for Liraglutide. *p<0.05, **p<0.01, ***p<0.001

TABLE 3.11

OGTT Plasma insulin (ng/mL) AUC above baseline

| Treatment | n | Mean | SEM | Difference | p |
|---|---|---|---|---|---|
| AUC above baseline (0-60 mins) | | | | | |
| Vehicle (de-ionised water) po | 11 | 3.32 | 0.49 | | |
| Compound 1 - 0.3 mg/kg po | 12 | 2.34 | 0.14 | -0.98 | 0.003** |
| Compound 1 - 1 mg/kg po | 12 | 1.20 | 0.30 | -2.12 | <0.001*** |
| Compound 1 - 3 mg/kg po | 12 | 1.05 | 0.18 | -2.27 | <0.001*** |
| Compound 1 - 10 mg/kg po | 12 | 1.30 | 0.18 | -2.01 | <0.001*** |
| Liraglutide 0.2 mg/kg sc | 11 | 1.92 | 0.31 | -1.40 | <0.001*** |

AUC above baseline (0-120 mins)

| | | | | | |
|---|---|---|---|---|---|
| Vehicle (de-ionised water) po | 11 | 3.42 | 0.55 | | |
| Compound 1 - 0.3 mg/kg po | 12 | 2.76 | 0.16 | -0.66 | 0.170 |
| Compound 1 - 1 mg/kg po | 12 | 1.80 | 0.50 | -1.62 | 0.001** |
| Compound 1 - 3 mg/kg po | 12 | 1.50 | 0.35 | -1.92 | 0.001** |
| Compound 1 - 10 mg/kg po | 12 | 2.05 | 0.25 | -1.37 | 0.001** |
| Liraglutide 0.2 mg/kg sc | 11 | 1.99 | 0.36 | -1.43 | 0.005** |

Means are adjusted for bleeding order and differences between the treatment groups in body weight on Day 1. Standard errors of the mean (SEM) are calculated from the residuals of the statistical model. Analysis was by robust regression of untransformed data. Comparisons to vehicle were by Williams’ test for Compound 1 - and the multiple t test for Liraglutide. **p<0.01, ***p<0.001

TABLE 3.12

Liver weights (mg)

| Treatment | n | Mean | SEM | Difference | % change | p |
|---|---|---|---|---|---|---|
| Liver (mg) | | | | | | |
| Vehicle (de-ionised water) po | 12 | 1735 | 73 | | | |
| Compound 1 - 0.3 mg/kg po | 12 | 1515 | 68 | -220 | -12.7 | 0.014* |
| Compound 1 - 1 mg/kg po | 12 | 1429 | 58 | -306 | -17.6 | <0.001*** |
| Compound 1 - 3 mg/kg po | 12 | 1311 | 38 | -424 | -24.5 | <0.001*** |
| Compound 1 - 10 mg/kg po | 12 | 1333 | 72 | -402 | -23.2 | <0.001*** |
| Liraglutide 0.2 mg/kg sc | 11 | 1205 | 52 | -530 | -30.5 | <0.001*** |

Means are adjusted for differences between the treatment groups in Day 1 bodyweight. Standard errors of the mean (SEM) are calculated from the residuals of the statistical model. Comparisons to vehicle were by Williams’ test for Compound 1 - and the multiple t test for Liraglutide. *p<0.05, ***p<0.001

TABLE 3.13

Liver lipid data

| Treatment | n | Mean | SEM | % | p |
|---|---|---|---|---|---|
| Triglycerides (umoles/g) | | | | | |
| Vehicle (de-ionised water) po | 12 | 108.9 | 28.7 | | |
| Compound 1 - 0.3 mg/kg po | 12 | 96.2 | 11.7 | 88.4 | 0.898 |
| Compound 1 - 1 mg/kg po | 12 | 106.9 | 24.4 | 98.2 | 0.898 |
| Compound 1 - 3 mg/kg po | 12 | 77.7 | 16.9 | 71.3 | 0.339 |
| Compound 1 - 10 mg/kg po | 12 | 90.5 | 17.7 | 83.1 | 0.339 |
| Liraglutide 0.2 mg/kg sc | 11 | 35.1 | 5.8 | 32.3 | <0.001*** |
| Triglycerides (µmoles total) | | | | | |
| Vehicle (de-ionised water) po | 12 | 178.5 | 48.2 | | |
| Compound 1 - 0.3 mg/kg po | 12 | 139.3 | 18.1 | 78.0 | 0.504 |
| Compound 1 - 1 mg/kg po | 12 | 151.3 | 36.0 | 84.8 | 0.504 |
| Compound 1 - 3 mg/kg po | 12 | 101.1 | 21.9 | 56.6 | 0.072 |
| Compound 1 - 10 mg/kg po | 12 | 117.9 | 26.3 | 66.1 | 0.072 |
| Liraglutide 0.2 mg/kg sc | 11 | 42.6 | 6.7 | 23.8 | <0.001*** |
| Cholesterol (mg/g) | | | | | |
| Vehicle (de-ionised water) po | 12 | 3.71 | 0.26 | | |
| Compound 1 - 0.3 mg/kg po | 12 | 3.68 | 0.18 | 99.4 | >0.999 |
| Compound 1 - 1 mg/kg po | 12 | 4.12 | 0.36 | 111.2 | 0.471 |
| Compound 1 - 3 mg/kg po | 12 | 3.93 | 0.18 | 105.9 | 0.471 |
| Compound 1 - 10 mg/kg po | 12 | 3.93 | 0.41 | 106.0 | 0.471 |
| Liraglutide 0.2 mg/kg sc | 11 | 3.40 | 0.21 | 91.7 | 0.293 |

Cholesterol (mg total)

| | | | | | |
|---|---|---|---|---|---|
| Vehicle (de-ionised water) po | 12 | 6.07 | 0.31 | | |
| Compound 1 - 0.3 mg/kg po | 12 | 5.32 | 0.36 | 87.7 | 0.519 |
| Compound 1 - 1 mg/kg po | 12 | 5.94 | 0.53 | 97.9 | 0.519 |
| Compound 1 - 3 mg/kg po | 12 | 5.07 | 0.30 | 83.6 | 0.136 |
| Compound 1 - 10 mg/kg po | 12 | 5.28 | 0.57 | 87.1 | 0.136 |
| Liraglutide 0.2 mg/kg sc | 11 | 3.99 | 0.26 | 65.8 | <0.001*** |

Means are back-transformed and adjusted for differences between the treatment groups in body weight on Day 1. Standard errors of the mean (SEM) are calculated from the residuals of the statistical model. Comparisons to vehicle were by Williams’ test for Compound 1 - and the multiple t test for Liraglutide. ***p<0.001

### Example 4: Gastric Emptying in Lean C57BL/6J Mice

### Effects of Compound 1 on Gastric Emptying in Lean C57BL/6J Mice

The aim of this study was to test the effects of oral Compound 1 administration on the rate of gastric emptying using the acetaminophen absorption test in lean male C57BL6/JRj mice. This assay is a clinically established method for the assessment of gastric emptying.

### Materials and Methods

Animals: Sixty male C57BL/6JRj mice (weight range 23-25 g) were ordered from Janvier, France.

Experimental Procedure: Chow-fed male C57BL/6JRj mice (8 weeks of age) were acclimatized for 6 days prior to study start. On study day -1 animals were randomized into 6 treatment groups (n=10 per group) based on body weight and semi-fasted by offering 50% of their average food intake. The following morning (day 1, t=-30 min) animals were PO dosed with vehicle or Compound 1 (0.3, 1, 3 or 10 mg/kg). Semaglutide (10 nmol/kg) was administered SC in one group and used as positive assay control. At t=0 min, acetaminophen solution was administered (160 mg/kg, PO). Blood samples for acetaminophen determination was collected at t= 10, 30, 60 and 120 min.

### Results

Compared to vehicle, Compound 1 inhibited gastric emptying in a dose-dependent manner at 10-, 30- and 60-min post acetaminophen dosing. All four doses of Compound 1, 0.3, 1, 3 and 1 mg/kg, showed a significantly lower serum acetaminophen (AUCO-120 min) compared to vehicle. Semaglutide (10 nmol/kg) inhibited gastric emptying at 10, 30 and 60 min. post acetaminophen dosing and had a lower serum acetaminophen AUCO-120 min compared to vehicle. In conclusion, Compound 1 demonstrated inhibitory effects on gastric emptying in lean mice. This represents one of the mechanisms by which acute dosing with our TAAR1 agonist, Compound 1, reduces glucose excursion in response to an oral glucose challenge. The results are shown in Table 4.1 below:

TABLE 4.1

Serum acetaminophen concentrations - AUC (0-120 min)

| Treatment | n | Mean | SEM | P |
|---|---|---|---|---|
| Vehicle po | 10 | 36568 | 1504 | |
| Compound 1 - 0.3 mg/kg po | 10 | 27476 | 1341 | <0.01** |
| Compound 1 - 1 mg/kg po | 10 | 19847 | 1021 | <0.001*** |
| Compound 1 - 3 mg/kg po | 10 | 15167 | 1893 | <0.001*** |
| Compound 1 - 10 mg/kg po | 10 | 13316 | 1287 | <0.001*** |
| Semaglutide 10 nmol/kg, sc | 10 | 27552 | 2363 | <0.01** |

Values expressed as mean of n = 10 + SEM. Dunnett’s test one-factor linear model. **: P < 0.01, ***: P < 0.001 compared to Vehicle.

### Example 5: Effects of Morning Dose of Compound 1 on ECG Intervals and Metabolic Effects in Human Subjects With Schizophrenia

The aim of this study was to test the effects of single dose administration of oral Compound 1 on electrocardiogram (ECG) intervals in adult human subjects with schizophrenia. The primary objective was to evaluate the QT interval corrected for heart rate (QTc) effects, while the secondary objective was to evaluate the electrocardiographic effects, both of these compared to placebo and using moxifloxacin as an active control. The final objective was to assess the safety and tolerability of a single dose of Compound 1 in adults with schizophrenia and to assess the metabolic effect.

Methodology: This was a Phase 1, randomized, single-dose, active- and placebo-controlled, 3-period crossover study of the effects of Compound 1 150 mg on ECG intervals in subjects with schizophrenia. Compound 1 and matching placebo were utilized in a double-blind fashion. Moxifloxacin was utilized as an active control in an open-label fashion.

Prior to dosing in Period 1, subjects were randomly assigned on Day 1 to one of the 6 sequences per double Williams Design in which Compound 1 150 mg (blinded), placebo (blinded), and moxifloxacin 400 mg (open-label) were administered in different orders from Period 1 through Period 3. A 5-day washout period was given between any 2 doses.

Subjects: Male or female subjects, of any race, between 18 and 65 years of age (inclusive), with a body mass index (BMI) between 18.0 and 35.0 kg/m² (inclusive) were enrolled in the study. Subjects had to meet Diagnostic and Statistical Manual of Mental Disorders criteria for a primary diagnosis of schizophrenia as established by clinical interview. Sixty-eight subjects were enrolled, and sixty subjects completed the study.

Experimental Procedure: Compound 1 and matching placebo were administered in a double-blind fashion. A single dose of Compound 1 (150 mg) or matched placebo or moxifloxacin 400 mg was administered orally in the morning. Data from continuous 12-lead ECG Holter monitoring, standard digital 12-lead ECGs for safety assessment, and blood samples were collected.

### Results

Compound 1 at 150 mg had no clinically relevant effect on heart rate or cardiac conduction, i.e., PR interval, and QRS duration. There were no positive findings on the C-SSRS at Day -2, baseline, postbaseline, and follow-up visits. There were no deaths. Overall, most AEs were assessed by the Investigator as mild or moderate in severity: 32 (50.8%) subjects receiving Compound 1 experienced 70 AEs, 8 (12.7%) subjects receiving moxifloxacin experienced 8 AEs, and 2 (3.2%) subjects receiving placebo experienced 3 AEs. The most common (≥ 5% subjects) AEs in subjects receiving Compound 1 were nausea and somnolence (16 [25.4%] subjects each), dizziness and vomiting (7 [11.1%] subjects each), and diarrhea (4 [6.3%] subjects). One SAE (hypotension) in one subject was assessed as severe and related to Compound 1, and one subject receiving moxifloxacin experienced severe schizophrenia (Investigator term: worsening of psychosis secondary to schizophrenia). Compound 1 at 150 mg was generally well-tolerated in adults with schizophrenia in this study.

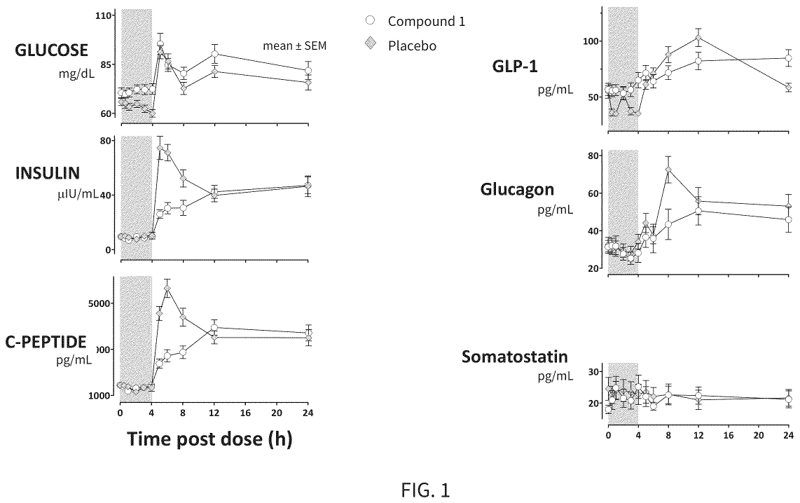

Metabolic effects of Compound 1 were investigated. FIG. 1 illustrates the metabolic effect of Compound 1, in response to a meal. At time zero, 150 mg of Compound 1 was administered to human subjects. Subjects were fasted overnight and dosed in the morning followed by additional 4 hours of fasting (i.e., the shaded area in FIG. 1). Median peak concentration of Compound 1 in the plasma was 468 ng/mL (mean Cₘₐₓ) occurring at median time 3.60 hours post-dose. Plasma samples were analyzed using Multiplexing (U-plex MSD) assay to measure C-peptide, Insulin, Glucagon, and GLP-1, using a separate oxidase assay to measure glucose, and using a separate ELISA assay to measure somatostatin. Each subject received Compound 1 and placebo with a between-period washout of 5 days.

Following administration of a meal, Compound 1 significantly lowered insulin and C-peptide levels, and dampened the glucose response compared to placebo, indicating an effect of Compound 1 on glycemic control in response to feeding (as can be seen in FIG. 1). Compound 1 also affected GLP-1 and glucagon levels eight hours after the meal was consumed. No effect was detected on somatostatin levels.

### Example 6: Intravenous Glucose Tolerance Test (IVGGT) Studies

### Effect of Compound 1 on Glucose Tolerance in Lean Male CDI Mice

This study aimed to evaluate the effects of administration of Compound 1 on glucose control in lean, fasted, male CD1 mice. An acute intravenous glucose tolerance test (IVGTT) was performed following an overnight fast with Compound 1 being administered at four doses (0.3, 1, 3 and 10 mg/kg po) 30 minutes prior to the glucose load. A vehicle control group was included in the study design. In addition, the GLP-1 receptor agonist, exendin-4 (40 µg/kg iv) was utilized as a positive control, with this group being dosed simultaneously to the glucose load. Blood samples were taken at various timepoints during the test and subsequently assessed for plasma glucose and insulin content.

### Materials and Methods

Animals and Animal Husbandry: Sixty-four (64) lean, male CD-1 mice (4 spares; 20-25 g) were purchased from Charles River, UK, Limited (Margate, Kent). Animal husbandry was generally as described above in Example 1.1.

Experimental Procedures: Experimental procedures were generally performed as described above in Example 1.1, except the glucose tolerance test was an IVGTT, rather than an oGTT. The groups for the study were: Group A - Vehicle (n = 10, po); Group B - Exendin-4 (40 µg/kg, ip)(n=10); Group C - Compound 1 (0.3 mg/kg, po)(n=10); Compound 1 (1 mg/kg, po)(n=10); Compound 1 (3 mg/kg, po)(n=10); Compound 1 (10 mg/kg, po)(n=10).

Following the overnight fast, animals underwent a glucose tolerance test via the intravenous route (IVGTT). Via a lateral tail vein, a baseline blood sample (B1; 30 µl) was taken from all animals prior to vehicle or drug treatment. Five minutes later, mice were dosed with Treatment 1 detailed by the oral route (see above table; approx. 30 minutes pre-glucose). Thirty minutes later, mice were dosed with Treatment 2 by the intravenous route (by a lateral tail vein). Blood samples were taken 5 (20 µl), 10 (20 µl), 30 (20 µl) and 60 (20 µl) minutes post-glucose challenge (assuming an 18 g mouse; samples were increased for larger animals). A B2 sample (30 µl) was also taken briefly before glucose administration. All blood samples (from the tail vein) were collected into lithium heparinised tubes (Sarstedt Microvette CB300LH) and plasma separated by centrifugation to produce a single aliquot of plasma which was frozen (approx. -80° C.) and subsequently assayed for glucose (Thermoelectron infinity glucose reagent, TR15421) and insulin (Alpco Ultrasensitive mouse insulin ELISA) as single replicates.

### Results

All animals were in good condition throughout the test and no adverse effects were evident at any stage of the protocol. Plasma glucose peaked from a baseline (B1) concentration of 6.12 mM to 30.69 mM at 5 minutes post-glucose load, subsequently reducing to 11.01 mM at 60 minutes post-glucose in the vehicle-treated control animals. Exendin-4 (40 µg/kg iv) produced a statistically significant (p<0.05) reduction in plasma glucose at 30 and 60 minutes post-glucose and similarly reduced overall AUC and AUCB2 (0-60 minutes) compared to vehicle-treated control animals (Tables 6.1 - 6.3).

The four doses of Compound 1 (0.3, 1, 3 and 10 mg/kg po) administered 30 minutes before the glucose load had no significant effect on glucose levels at any time point post the glucose load.

Exendin-4 produced a statistically significant increase in plasma insulin at 5, 10 (both p<0.001) and 30 minutes (p<0.05) post glucose load. Plasma insulin was increased from a baseline mean value of 0.17 ng/mL to a peak of 1.99 ng/mL at 5 minutes post glucose load. The mean plasma insulin level of exendin-4 treated animals was not significantly different to vehicle treated animals by 60 minutes post glucose load.

Compound 1 had the same profile on plasma insulin as it did on plasma glucose. Specifically, at the doses tested, plasma insulin was not significantly reduced compared to vehicle-treated control counterparts after the glucose load and in all AUC calculations (Tables 6.4 - 6.6).

As expected, intravenous exendin-4 improved glucose control in the animals and this was associated with a transient increase in plasma insulin consistent with the known effect of the molecule to promote glucose-stimulated insulin secretion. Oral administration of Compound 1 had no statistically significant effect on plasma glucose or insulin after intravenous glucose administration.

Acute treatment with Compound 1 (0.3, 1, 3 and 10 mg/kg po) had no significant effect on plasma glucose and insulin excursion after an intravenous glucose load compared to vehicle-treated controls over the 60-minute period. The GLP-1 receptor agonist, exendin-4 (40 µg/kg iv), significantly reduced plasma glucose and increased plasma insulin compared to vehicle-treated control mice when co-administered with the glucose.

The absence of an effect of Compound 1 on glucose excursion during an ivGTT further confirms that the improved glucose-tolerance in the oGTT (see example 1.1. above) is mediated by a delay in gastric emptying.

TABLE 6.1

Plasma glucose (mM)

| Treatment | n | Mean | SEM | % of vehicle | p |
|---|---|---|---|---|---|
| Pre-treatment baseline (B1) | | | | | |
| All treatments | 60 | 6.12 | 0.00 | | |
| Post-treatment baseline (B2) | | | | | |
| Vehicle po + Glucose 1 g/kg iv | 10 | 9.43 | 0.46 | | |
| Vehicle po + Glucose 1 g/kg, Exendin-4 40 µg/kg iv | 10 | 9.30 | 0.81 | 98.6 | 0.873 |
| Compound 1 - 0.3 mg/kg po + Glucose 1 g/kg iv | 10 | 10.18 | 0.45 | 107.9 | 0.661 |
| Compound 1 - 1 mg/kg po + Glucose 1 g/kg iv | 10 | 9.92 | 0.40 | 105.1 | 0.661 |
| Compound 1 - 3 mg/kg po + Glucose 1 g/kg iv | 10 | 9.92 | 0.54 | 105.2 | 0.661 |
| Compound 1 - 10 mg/kg po + Glucose 1 g/kg iv | 10 | 10.61 | 0.63 | 112.4 | 0.264 |

TABLE 6.1

Plasma glucose (mM)

| Treatment | n | Mean | SEM | % of vehicle | p |
|---|---|---|---|---|---|
| 5 minutes | | | | | |
| Vehicle po + Glucose 1 g/kg iv | 10 | 30.69 | 0.42 | | |
| Vehicle po + Glucose 1 g/kg, Exendin-4 40 µg/kg iv | 10 | 28.43 | 0.93 | 92.6 | 0.085 |
| Compound 1 - 0.3 mg/kg po + Glucose 1 g/kg iv | 10 | 30.44 | 0.88 | 99.2 | 0.849 |
| Compound 1 - 1 mg/kg po + Glucose 1 g/kg iv | 10 | 29.57 | 0.62 | 96.4 | 0.479 |
| Compound 1 - 3 mg/kg po + Glucose 1 g/kg iv | 10 | 28.19 | 0.95 | 91.8 | 0.470 |
| Compound 1 - 10 mg/kg po + Glucose 1 g/kg iv | 10 | 30.80 | 1.00 | 100.3 | 0.470 |
| 10 minutes | | | | | |
| Vehicle po + Glucose 1 g/kg iv | 10 | 24.79 | 0.99 | | |
| Vehicle po + Glucose 1 g/kg, Exendin-4 40 µg/kg iv | 10 | 22.15 | 1.54 | 89.4 | 0.059 |
| Compound 1 - 0.3 mg/kg po + Glucose 1 g/kg iv | 10 | 27.52 | 1.02 | 111.0 | 0.363 |
| Compound 1 - 1 mg/kg po + Glucose 1 g/kg iv | 10 | 26.03 | 0.86 | 105.0 | 0.363 |
| Compound 1 - 3 mg/kg po + Glucose 1 g/kg iv | 10 | 25.65 | 1.00 | 103.5 | 0.363 |
| Compound 1 - 10 mg/kg po + Glucose 1 g/kg iv | 10 | 26.59 | 1.09 | 107.3 | 0.307 |
| 30 minutes | | | | | |
| Vehicle po + Glucose 1 g/kg iv | 10 | 14.16 | 1.87 | | |
| Vehicle po + Glucose 1 g/kg, Exendin-4 40 µg/kg iv | 10 | 8.35 | 0.75 | 59.0 | 0.013* |
| Compound 1 - 0.3 mg/kg po + Glucose 1 g/kg iv | 10 | 12.44 | 1.76 | 87.9 | >0.999 |
| Compound 1 - 1 mg/kg po + Glucose 1 g/kg iv | 10 | 14.28 | 1.91 | 100.8 | >0.999 |
| Compound 1 - 3 mg/kg po + Glucose 1 g/kg iv | 10 | 13.79 | 2.87 | 97.4 | >0.999 |
| Compound 1 - 10 mg/kg po + Glucose 1 g/kg iv | 10 | 16.88 | 2.42 | 119.2 | 0.527 |
| 60 minutes | | | | | |
| Vehicle po + Glucose 1 g/kg iv | 10 | 11.01 | 0.75 | | |
| Vehicle po + Glucose 1 g/kg, Exendin-4 40 µg/kg iv ∗∗ | 10 | 6.36 | 0.26 | 57.8 | <0.001* |
| Compound 1 - 0.3 mg/kg po + Glucose 1 g/kg iv | 10 | 11.08 | 0.80 | 100.7 | >0.999 |
| Compound 1 - 1 mg/kg po + Glucose 1 g/kg iv | 10 | 10.90 | 0.78 | 99.0 | >0.999 |
| Compound 1 - 3 mg/kg po + Glucose 1 g/kg iv | 10 | 9.98 | 1.44 | 90.7 | >0.999 |
| Compound 1 - 10 mg/kg po + Glucose 1 g/kg iv | 10 | 12.57 | 1.23 | 114.2 | 0.416 |

10 minutes

| | | | | | |
|---|---|---|---|---|---|
| Vehicle po + Glucose 1 g/kg iv | 10 | 24.79 | 0.99 | | |
| Vehicle po + Glucose 1 g/kg, Exendin-4 40 µg/kg iv | 10 | 22.15 | 1.54 | 89.4 | 0.059 |
| Compound 1 - 0.3 mg/kg po + Glucose 1 g/kg iv | 10 | 27.52 | 1.02 | 111.0 | 0.363 |
| Compound 1 - 1 mg/kg po + Glucose 1 g/kg iv | 10 | 26.03 | 0.86 | 105.0 | 0.363 |
| Compound 1 - 3 mg/kg po + Glucose 1 g/kg iv | 10 | 25.65 | 1.00 | 103.5 | 0.363 |
| Compound 1 - 10 mg/kg po + Glucose 1 g/kg iv | 10 | 26.59 | 1.09 | 107.3 | 0.307 |

30 minutes

| | | | | | |
|---|---|---|---|---|---|
| Vehicle po + Glucose 1 g/kg iv | 10 | 14.16 | 1.87 | | |
| Vehicle po + Glucose 1 g/kg, Exendin-4 40 µg/kg iv | 10 | 8.35 | 0.75 | 59.0 | 0.013* |
| Compound 1 - 0.3 mg/kg po + Glucose 1 g/kg iv | 10 | 12.44 | 1.76 | 87.9 | >0.999 |
| Compound 1 - 1 mg/kg po + Glucose 1 g/kg iv | 10 | 14.28 | 1.91 | 100.8 | >0.999 |
| Compound 1 - 3 mg/kg po + Glucose 1 g/kg iv | 10 | 13.79 | 2.87 | 97.4 | >0.999 |
| Compound 1 - 10 mg/kg po + Glucose 1 g/kg iv | 10 | 16.88 | 2.42 | 119.2 | 0.527 |

60 minutes

| | | | | | |
|---|---|---|---|---|---|
| Vehicle po + Glucose 1 g/kg iv | 10 | 11.01 | 0.75 | | |
| Vehicle po + Glucose 1 g/kg iv, Exendin-4 40 µg/kg iv | 10 | 6.36 | 0.26 | 57.8 | <0.001** * |
| Compound 1 - 0.3 mg/kg po + Glucose 1 g/kg iv | 10 | 11.08 | 0.80 | 100.7 | >0.999 |
| Compound 1 - 1 mg/kg po + Glucose 1 g/kg iv | 10 | 10.90 | 0.78 | 99.0 | >0.999 |
| Compound 1 - 3 mg/kg po + Glucose 1 g/kg iv | 10 | 9.98 | 1.44 | 90.7 | >0.999 |
| Compound 1 - 10 mg/kg po + Glucose 1 g/kg iv | 10 | 12.57 | 1.23 | 114.2 | 0.416 |

Means are back-transformed and adjusted for differences between the treatment groups in bleeding order, baseline body weight and pre-treatment baseline (B1) glucose. Standard errors of the mean (SEM) are calculated from the residuals of the statistical model. Analysis was by robust regression of log transformed data. Comparisons to vehicle were by multiple t test for Exendin-4 and by Williams’ test for Compound 1 -. *p<0.05, ***p<0.001.

TABLE 6.2

Plasma glucose AUC (mM.hr)

| Treatment | n | Mean | SEM | % of vehicle | p |
|---|---|---|---|---|---|
| Area under curve (0-60 minutes) | | | | | |
| Vehicle po + Glucose 1 g/kg iv | 10 | 17.22 | 0.88 | | |
| Vehicle po + Glucose 1 g/kg, Exendin-4 40 | 10 | 12.65 | 0.63 | 73.5 | <0.001*** |
| µg/kg iv | | | | | |
| Compound 1 - 0.3 mg/kg po + Glucose 1 g/kg iv | 10 | 17.23 | 0.97 | 100.1 | >0.999 |
| Compound 1 - 1 mg/kg po + Glucose 1 g/kg iv | 10 | 16.96 | 0.99 | 98.5 | >0.999 |
| Compound 1 - 3 mg/kg po + Glucose 1 g/kg iv | 10 | 16.50 | 1.24 | 95.9 | >0.999 |
| Compound 1 - 10 mg/kg po + Glucose 1 g/kg iv | 10 | 18.86 | 1.01 | 109.6 | 0.369 |

Means are back-transformed and adjusted for differences between the treatment groups in bleeding order, baseline body weight and pre-treatment baseline (B1) glucose. Standard errors of the mean (SEM) are calculated from the residuals of the statistical model. Analysis was by robust regression of log transformed data. Comparisons to vehicle were by multiple t test for Exendin-4 and by Williams’ test for Compound 1 -. ***p<0.001.

TABLE 6.3

Plasma glucose (mM.hr) - AUC above baseline (AUCB2)

| Treatment | n | Mean | SEM | Difference from vehicle | p |
|---|---|---|---|---|---|
| AUC above baseline (0-60 minutes) | | | | | |
| Vehicle po + Glucose 1 g/kg iv | 10 | 7.65 | 1.07 | | |
| Vehicle po + Glucose 1 g/kg, Exendin-4 40 µg/kg iv | 10 | 3.22 | 0.88 | -4.43 | 0.005** |
| Compound 1 - 0.3 mg/kg po + Glucose 1 g/kg iv | 10 | 7.18 | 0.81 | -0.47 | >0.999 |
| Compound 1 - 1 mg/kg po + Glucose 1 g/kg iv | 10 | 7.12 | 1.28 | -0.53 | >0.999 |
| Compound 1 - 3 mg/kg po + Glucose 1 g/kg iv | 10 | 6.40 | 1.13 | -1.25 | >0.999 |
| Compound 1 - 10 mg/kg po + Glucose 1 g/kg iv | 10 | 8.83 | 0.80 | 1.18 | 0.576 |

Means are adjusted for differences between the treatment groups in bleeding order, baseline body weight and pre-treatment baseline (B1) glucose. Standard errors of the mean (SEM) are calculated from the residuals of the statistical model. Analysis was by robust regression of untransformed data. Comparisons to vehicle were by multiple t test for Exendin-4 and by Williams’ test for Compound 1 -. **p<0.01

TABLE 6.4

Plasma insulin (ng/mL)

| Treatment | n | Mean | SEM | % of vehicle | p |
|---|---|---|---|---|---|
| Pre-treatment baseline Dav 1 (B1) | | | | | |
| All treatments | 60 | 0.09 | 0.00 | | |
| Post-treatment baseline (B2) | | | | | |
| Vehicle po + Glucose 1 g/kg iv | 10 | 0.18 | 0.08 | | |
| Vehicle po + Glucose 1 g/kg, Exendin-4 40 µg/kg iv | 10 | 0.17 | 0.08 | 95.1 | 0.872 |
| Compound 1 - 0.3 mg/kg po + Glucose 1 g/kg iv | 10 | 0.23 | 0.04 | 126.4 | >0.999 |
| Compound 1 - 1 mg/kg po + Glucose 1 g/kg iv | 10 | 0.14 | 0.06 | 78.9 | 0.666 |
| Compound 1 - 3 mg/kg po + Glucose 1 g/kg iv | 10 | 0.15 | 0.07 | 83.0 | 0.666 |
| Compound 1 - 10 mg/kg po + Glucose 1 g/kg iv | 10 | 0.14 | 0.03 | 80.9 | 0.666 |
| 5 minutes | | | | | |
| Vehicle po + Glucose 1 g/kg iv | 10 | 0.57 | 0.11 | | |
| Vehicle po + Glucose 1 g/kg, Exendin-4 40 µg/kg iv | 10 | 1.99 | 0.29 | 347.3 | <0.001*** |
| Compound 1 - 0.3 mg/kg po + Glucose 1 g/kg iv | 10 | 0.57 | 0.10 | 99.3 | 0.983 |
| Compound 1 - 1 mg/kg po + Glucose 1 g/kg iv | 10 | 0.46 | 0.09 | 80.8 | 0.604 |
| Compound 1 - 3 mg/kg po + Glucose 1 g/kg iv | 10 | 0.38 | 0.13 | 65.8 | 0.244 |
| Compound 1 - 10 mg/kg po + Glucose 1 g/kg iv | 10 | 0.30 | 0.14 | 52.5 | 0.060 |

10 minutes

| | | | | | |
|---|---|---|---|---|---|
| Vehicle po + Glucose 1 g/kg iv | 10 | 0.56 | 0.09 | | |
| Vehicle po + Glucose 1 g/kg, Exendin-4 40 µg/kg iv | 10 | 1.84 | 0.19 | 327.2 | <0.001*** |
| Compound 1 - 0.3 mg/kg po + Glucose 1 g/kg iv | 10 | 0.58 | 0.05 | 103.4 | >0.999 |
| Compound 1 - 1 mg/kg po + Glucose 1 g/kg iv | 10 | 0.54 | 0.09 | 96.6 | >0.999 |
| Compound 1 - 3 mg/kg po + Glucose 1 g/kg iv | 10 | 0.55 | 0.05 | 97.7 | >0.999 |
| Compound 1 - 10 mg/kg po + Glucose 1 g/kg iv | 10 | 0.47 | 0.23 | 83.0 | 0.453 |

30 minutes

| | | | | | |
|---|---|---|---|---|---|
| Vehicle po + Glucose 1 g/kg iv | 10 | 0.38 | 0.16 | | |
| Vehicle po + Glucose 1 g/kg, Exendin-4 40 µg/kg iv | 10 | 0.57 | 0.05 | 149.8 | 0.037* |
| Compound 1 - 0.3 mg/kg po + Glucose 1 g/kg iv | 10 | 0.43 | 0.03 | 113.1 | >0.999 |
| Compound 1 - 1 mg/kg po + Glucose 1 g/kg iv | 10 | 0.31 | 0.05 | 82.3 | 0.921 |
| Compound 1 - 3 mg/kg po + Glucose 1 g/kg iv | 10 | 0.39 | 0.04 | 103.0 | 0.921 |
| Compound 1 - 10 mg/kg po + Glucose 1 g/kg iv | 10 | 0.36 | 0.20 | 94.7 | 0.921 |

60 minutes

| | | | | | |
|---|---|---|---|---|---|
| Vehicle po + Glucose 1 g/kg iv | 10 | 0.36 | 0.06 | | |
| Vehicle po + Glucose 1 g/kg, Exendin-4 40 µg/kg iv | 10 | 0.42 | 0.04 | 117.2 | 0.232 |
| Compound 1 - 0.3 mg/kg po + Glucose 1 g/kg iv | 10 | 0.41 | 0.02 | 112.3 | 0.596 |
| Compound 1 - 1 mg/kg po + Glucose 1 g/kg iv | 10 | 0.38 | 0.04 | 106.5 | 0.596 |
| Compound 1 - 3 mg/kg po + Glucose 1 g/kg iv | 10 | 0.45 | 0.03 | 123.4 | 0.179 |
| Compound 1 - 10 mg/kg po + Glucose 1 g/kg iv | 10 | 0.43 | 0.09 | 120.4 | 0.179 |

Means are back-transformed and adjusted for differences between the treatment groups in bleeding order, baseline body weight and pre-treatment baseline (B1) insulin. Standard errors of the mean (SEM) are calculated from the residuals of the statistical model. Analysis was by robust regression of log transformed data. Comparisons to vehicle were by multiple t test for Exendin-4 and by Williams’ test for Compound 1 -. *p<0.05, ***p<0.001.

TABLE 6.5

Plasma insulin (ng/mL.hr) - Area under curve AUC

| Treatment | n | Mean | SEM | % of vehicle | p |
|---|---|---|---|---|---|
| Area under curve (0-60 minutes) | | | | | |
| Vehicle po + Glucose 1 g/kg iv | 10 | 0.43 | 0.06 | | |
| Vehicle po + Glucose 1 g/kg, Exendin-4 40 µg/kg iv | 10 | 0.93 | 0.08 | 218.8 | <0.001*** |
| Compound 1 - 0.3 mg/kg po + Glucose 1 g/kg iv | 10 | 0.48 | 0.02 | 112.0 | >0.999 |
| Compound 1 - 1 mg/kg po + Glucose 1 g/kg iv | 10 | 0.38 | 0.05 | 89.6 | 0.974 |
| Compound 1 - 3 mg/kg po + Glucose 1 g/kg iv | 10 | 0.44 | 0.03 | 103.4 | 0.974 |
| Compound 1 - 10 mg/kg po + Glucose 1 g/kg iv | 10 | 0.40 | 0.13 | 94.6 | 0.893 |

Means are back-transformed and adjusted for differences between the treatment groups in bleeding order, baseline body weight and pre-treatment baseline (B1) insulin.
Standard errors of the mean (SEM) are calculated from the residuals of the statistical model. Analysis was by robust regression of log transformed data.
Comparisons to vehicle were by multiple t test for Exendin-4 and by Williams’ test for Compound 1 -. ***p<0.001.

TABLE 6.6

Plasma insulin (ng/mL.hr) - AUC above baseline (AUCB2)

| Treatment | n | Mean | SEM | Difference from vehicle | p |
|---|---|---|---|---|---|
| AUC above baseline (0-60 minutes) | | | | | |
| Vehicle po + Glucose 1 g/kg iv | 10 | 0.25 | 0.04 | | |
| Vehicle po + Glucose 1 g/kg, Exendin-4 40 µg/kg iv | 10 | 0.73 | 0.11 | 0.48 | <0.001*** |
| Compound 1 - 0.3 mg/kg po + Glucose 1 g/kg iv | 10 | 0.26 | 0.03 | 0.00 | >0.999 |
| Compound 1 - 1 mg/kg po + Glucose 1 g/kg iv | 10 | 0.23 | 0.05 | -0.02 | >0.999 |
| Compound 1 - 3 mg/kg po + Glucose 1 g/kg iv | 10 | 0.29 | 0.06 | 0.04 | >0.999 |
| Compound 1 - 10 mg/kg po + Glucose 1 g/kg iv | 10 | 0.24 | 0.04 | -0.02 | >0.999 |

Means are adjusted for differences between the treatment groups in bleeding order, baseline body weight and pre-treatment baseline (B1) insulin.
Standard errors of the mean (SEM) are calculated from the residuals of the statistical model. Analysis was by robust regression of untransformed data.
Comparisons to vehicle were by multiple t test for Exendin-4 and by Williams’ test for Compound 1 -. ***p<0.001.

### Example 7: Phase 1 Study on Gastric Retention and Emptying in Humans

### A Randomized, Open Label, Single Dose, Cross-Over Study of Gastric Emptying Rate: Compound 1 vs Prior Antipsychotic (PA) Standard of Care in Subjects with Schizophrenia

The aim of this study is to examine the effect of Compound 1 on gastric retention and emptying in subjects with schizophrenia suffering from metabolic dysregulation and to provide a possible mechanistic explanation for the effects of Compound 1 on glucose regulation.

Study Design: This is a randomized, open-label, single dose two-period crossover study with two treatment sequences. For each treatment sequence, subjects receive a single dose of Compound 1 and prior antipsychotic (PA) standard of care in random order.

Subjects: Male or female subjects, of any race, between 18 and 65 years of age (inclusive), with a history of BMI ≤ 25 kg/m²prior to Schizophrenia diagnosis, are eligible for the study. Subjects must meet Diagnostic and Statistical Manual of Mental Disorders (DSM-5) criteria for a primary diagnosis of schizophrenia as established by clinical interview, using the DSM-5 as a reference and confirmed using the Structured Clinical Interview for DSM-5, Clinical Trials Version [SCID-CT]), and must be receiving risperidone, olanzapine, quetiapine or aripiprazole as treatment for schizophrenia at the time of Screening. Subject must also have a CGI-S score ≤ 4 (normal to moderately ill) at screening, and have normal to mild symptoms on all individual items of SAS (< 2), AIMS (< 3) and BARS (< 3) at screening.

At screening, the subject must fulfill metabolic eligibility criteria 1:

The subject must have any three of the following metabolic syndrome criteria:
a) waist circumference:
   ≥ 40 inches (101.6 cm) for men
   ≥ 35 inches (88.9 cm) for women
b) triglyceride concentration ≥ 150 mg/dL (1.69 mmol/L) or ongoing treatment with triglyceride lowering medication
c) HDL-cholesterol concentration:
   < 40 mg/dL (1.03 mmol/L) for men
   < 50 mg/dL (1.29 mmol/L) for women
   or ongoing treatment with cholesterol lowering medication
   d) fasting glucose concentrations ≥ 100 mg/dL (5.6 mmol/L)
   e) either standing or supine blood pressure ≥ 130/85 mmHg or ongoing treatment with antihypertensive medication

At screening, subject must fulfill metabolic eligibility criteria 2:

The subject must demonstrate glycemic derived insulin resistance as evidenced by one of the following criteria:
a) 5.7% ≤ HbAlc ≤ 6.4%
b) Fasting HOMA-IR ([Insulin uIU/ml x Glucose mg/dl] / 405) ≥ 2.22

### Experimental Procedure

Following screening evaluations, subjects will check-in to the clinical research unit. After baseline assessments, each cohort of subjects will be randomized to Compound 1 or continue on their PA. Wash out of the oral psychotropic medication (or any other medication with psychotropic propensity) will occur Day -16 to Day -1 (depending on the antipsychotic medication half-life) for subjects randomized to Compound 1.
a. On the first dosing day, after the overnight fast and morning labs/assessments, subjects will receive either Compound 1 or their PA medication per randomization and will have a ^{99m}Tc-labeled meal with successive γ camera assessments over a 4-hour period of time post-meal. Subjects will then undergo a washout period.
b. On the second dosing day, after the overnight fast and morning labs/assessments, subjects will receive second period treatment: subjects who received Compound 1 in the first period will receive PA and subjects who received PA in the first period will receive Compound 1. After consuming ^{99m}Tc-labeled meal, subjects will undergo successive γ camera assessments over 4-hour period of time. Subjects will be stabilized (Up to 6 days, depending on the clinical assessment/need) and discharged. Follow up visit will be required 7 ± 2 days from discharge.

The primary endpoints of gastric emptying T_{½} and gastric retention at 4 hours, as well as the secondary endpoint of gastric retention at 2 hours, is measured. Adverse event incidence, changes in laboratory assessments, vital signs, 12-lead ECG parameters, and frequency and severity of suicidal ideation and suicidal behavior based on the C-SSRS are observed as safety endpoints.

### Example 8: Phase 1 Study on Weight Associated Parameters in Humans

### A Double-Blind, Randomized, Multiple Dose Study of Weight Associated Parameters: Compound 1 vs. Over-Encapsulated Prior Antipsychotic (PA) Standard of Care in Subjects With Schizophrenia Suffering From Metabolic Dysregulation

The aim of this study is to examine the effects of Compound 1 on insulin sensitivity (glucose rate of appearance, glucose rate of disappearance and glycerol rate of appearance) and on body composition (total body fat, abdominal fat and subcutaneous adiposity) in subjects with schizophrenia suffering from metabolic dysregulation. Other objectives of the study include evaluating the effect of Compound 1 on 1) biomarkers of metabolic effect; 2) fasting serum lipids, triglyceride/HDL ratio, Hb_{A1C}, waist circumference and body weight; and 3) PK/biomarkers.

Study Design: This is a double-blind, randomized, multiple dose study in male and female adult subjects with schizophrenia. 48 subjects are randomized in a 1:1 ratio to receive oral Compound 1 (up to total daily dose of 100 mg [up to 50 mg twice a day]) after a suitable washout period of their prior antipsychotic (PA) for 4 weeks. Titration up to the total 100 mg/day dose is included to improve tolerability. This study utilizes a twice a day dosing regimen (evening dosing with a meal and morning dosing with a meal).

Subjects: Male or female subjects, of any race, between 18 and 65 years of age (inclusive), with a history of BMI ≤ 25 kg/m² prior to Schizophrenia diagnosis, are eligible for the study. Subjects must meet Diagnostic and Statistical Manual of Mental Disorders (DSM-5) criteria for a primary diagnosis of schizophrenia as established by clinical interview, using the DSM-5 as a reference and confirmed using the Structured Clinical Interview for DSM-5, Clinical Trials Version [SCID-CT]), and must be receiving risperidone, olanzapine, quetiapine or aripiprazole as treatment for schizophrenia at the time of Screening. Subject must also have a CGI-S score ≤ 4 (normal to moderately ill) at screening, and have normal to mild symptoms on all individual items of SAS (< 2), AIMS (< 3) and BARS (< 3) at screening.

Subject must also have a positive and negative symptom scale (PANSS) total score ≤ 80 at Screening and a score of ≤ 4 on the following PANSS items at Screening: P7 (hostility) and G8 (uncooperativeness).

At screening, the subject must fulfill metabolic eligibility criteria 1:

The subject must have any three of the following metabolic syndrome criteria:
a) waist circumference:
   ≥ 40 inches (101.6 cm) for men
   ≥ 35 inches (88.9 cm) for women
b) triglyceride concentration ≥ 150 mg/dL (1.69 mmol/L) or ongoing treatment with triglyceride lowering medication
c) HDL-cholesterol concentration:
   < 40 mg/dL (1.03 mmol/L) for men
   < 50 mg/dL (1.29 mmol/L) for women
   or ongoing treatment with cholesterol lowering medication
a) fasting glucose concentrations ≥ 100 mg/dL (5.6 mmol/L)
b) either standing or supine blood pressure ≥ 130/85 mmHg or ongoing treatment with antihypertensive medication.

At screening, subject must fulfill metabolic eligibility criteria 2:

The subject must demonstrate glycemic derived insulin resistance as evidenced by one of the following criteria:
5.7% ≤ HbAlc ≤ 6.4%
Fasting HOMA-IR ([Insulin uIU/ml x Glucose mg/dl] / 405) ≥ 2.22.

Experimental Procedure: Subsequent to the baseline assessments, subjects undergo structural MRI scans as well as glucose clamp, followed by randomization. Subjects randomized to Compound 1 undergo blinded washout (duration dependent on the drug’s half-life) and subsequent Compound 1 titration for up to 16 days. Upon reaching the stable, well tolerated Compound 1 exposure, subjects continue on the same Compound 1 dosing regimen for 15 days. Subjects randomized to their prior antipsychotic (PA) remain on their current PA blinded (over encapsulated) treatment. Subjects randomized to their PA have a blinded pseudo washout/titration period.

MRI scans as well as glucose clamp are conducted at the end of the treatment phase. Subjects randomized to PA continue to receive their blinded outpatient over-encapsulated antipsychotic while subjects randomized to Compound 1 have blinded stabilization to their prior PA.

### Primary Endpoints

Insulin stimulated change in glucose disposal rate during the steady state of the low dose hyperinsulinemic euglycemic clamp (HEC), baseline to end of the treatment.

Insulin stimulated change in glucose disposal rate during the steady state of the high dose HEC, baseline to end of the treatment.

Change in abdominal subcutaneous adipose tissue, visceral adipose tissue, liver proton density fat fraction (PDFF), muscles and organs of interest, baseline on PA to end of the treatment.

### Safety Endpoints

The incidence of overall adverse events (AEs), serious AEs (SAEs), and AEs (or SAEs) leading to discontinuation.

Observed values and changes from Baseline on PA at each scheduled visit in laboratory assessments, vital signs, 12-lead ECG parameters.

Frequency and severity of suicidal ideation and suicidal behavior based on the C-SSRS.

### Other Endpoints

Change in multiplex metabolic biomarkers glucose, c-peptide, insulin, and other potential biomarkers (including but not limited to glucagon, GLP 1), baseline on PA to each post-baseline timepoint.

Change in fasting serum lipids (VLDL), triglyceride/ HDL ratio, HbAlC, waist circumference and body weight, baseline on PA to end of the treatment.

Change in area under the curve (AUC) of 7-point glucose measurement.

### Example 9: Phase 1 Study on Glucose and Insulin Associated Parameters in Humans

### An Open Label, Fixed Sequence, Multiple Dose Study of Glucose and Insulin Associated Parameters: Compound 1 vs Prior Antipsychotic (PA) Standard of Care in Subjects With Schizophrenia Suffering From Metabolic Dysregulation

The aim of this study is to examine the effects of Compound 1 on glucose tolerance and on markers of β-cell function and incretins, as well as gastric emptying, in a population of patients with schizophrenia with metabolic dysregulation. Other objectives of the study include evaluating the effect of Compound 1 on gastric emptying and on visual analog scales of fullness, hunger and satiety. This study defines the individual subject exposure of Compound 1 at which modulation of biomarkers of metabolic effects occur.

### Primary Objectives

To evaluate the effect of Compound 1 on glucose tolerance.

To evaluate the effect of Compound 1 on markers of β-cell function and incretins.

### Secondary Objectives

To evaluate the effect of Compound 1 on gastric emptying.

To evaluate the effect of Compound 1 on visual analog scales of fullness, hunger and satiety.

### Safety Objectives

To evaluate the safety and tolerability of Compound 1.

### Other Objective

To measure the effects of Compound 1 on Homeostatic Model Assessment of Insulin Resistance (HOMA-IR) values.

### Primary Endpoints

Change from baseline (PA) in oGTT derived plasma AUC₀₋₁₂₀ min of glucose, insulin, c peptide in oGTT to stable dose (Compound 1) period assessment.

Change from baseline (PA) in mixed meal tolerance test (MMTT) derived plasma AUC₀₋₂₄₀ₘᵢₙ of glucose, insulin, c peptide, and Œ≤ cell responsivity index to stable dose (Compound 1) period assessment.

### Secondary Endpoint

Change from baseline (PA) in plasma AUC₀₋₂₄₀ₘᵢₙ and Cₘₐₓ of acetaminophen levels to stable dose (Compound 1) period assessment.

Change in gastric emptying terminal elimination half-life (T_{½}), kPCD and lag time, baseline to stable dose (Compound 1) period assessment.

Change in the Visual Analog Scale (VAS) of fullness, hunger and satiety, baseline -repeat test (PA baseline to Compound 1 stable dose period assessment) for all time points.

### Safety Endpoints

The incidence of overall AEs, serious AEs (SAEs), and AEs (or SAEs) leading to discontinuation.

Observed values and changes from Baseline (PA) laboratory assessments, vital signs, 12-lead ECG parameters.

Frequency and severity of suicidal ideation and suicidal behavior based on the C-SSRS.

### Other Endpoint

Observed values and changes from Baseline at each scheduled visit in HOMA-IR.

Study Design: This is an open-label, fixed sequence, multiple dose design study in male and female adult subjects with schizophrenia. 24 subjects receive oral tablets of Compound 1 (up to total daily dose of 100 mg [up to 50 mg twice a day]) for approximately two weeks. Titration up to the total 100 mg/day dose is included to improve tolerability. This study utilizes twice a day dosing regimen (evening dosing with a meal and morning dosing with a meal).

Subjects: Male or female subjects, of any race, between 18 and 65 years of age (inclusive), with a history of BMI ≤ 25 kg/m² prior to Schizophrenia diagnosis, are eligible for the study. Subjects must meet Diagnostic and Statistical Manual of Mental Disorders (DSM-5) criteria for a primary diagnosis of schizophrenia as established by clinical interview, using the DSM-5 as a reference and confirmed using the Structured Clinical Interview for DSM-5, Clinical Trials Version [SCID-CT]), and must be receiving risperidone, olanzapine, quetiapine or aripiprazole as treatment for schizophrenia at the time of Screening. Subject must also have a CGI-S score ≤ 4 (normal to moderately ill) at screening, and have normal to mild symptoms on all individual items of SAS (< 2), AIMS (< 3) and BARS (< 3) at screening.

Subject must also have a positive and negative symptom scale (PANSS) total score ≤ 80 at Screening and a score of ≤ 4 on the following PANSS items at Screening: P7 (hostility) and G8 (uncooperativeness).

At screening, the subject must fulfill metabolic eligibility criteria 1:

The subject must have any three of the following metabolic syndrome criteria:
d) waist circumference:
   ≥ 40 inches (101.6 cm) for men
   ≥ 35 inches (88.9 cm) for women
e) triglyceride concentration ≥150 mg/dL (1.69 mmol/L) or ongoing treatment with triglyceride lowering medication
f) HDL-cholesterol concentration:
   < 40 mg/dL (1.03 mmol/L) for men
   < 50 mg/dL (1.29 mmol/L) for women
   or ongoing treatment with cholesterol lowering medication
c) fasting glucose concentrations ≥100 mg/dL (5.6 mmol/L)
d) either standing or supine blood pressure ≥ 130/85 mmHg or ongoing treatment with antihypertensive medication.

At screening, subject must fulfill metabolic eligibility criteria 2:

The subject must demonstrate glycemic derived insulin resistance as evidenced by one of the following criteria:
5.7% ≤ HbA1c ≤ 6.4%
Fasting HOMA-IR ([Insulin Uiu/ml × Glucose mg/dl] / 405) ≥ 2.22.

Experimental Procedure: Subsequent to the baseline assessments, subjects undergo an oral glucose tolerance test (oGTT), mixed meal tolerance test (MMTT) and spirulina breath test (GEBT), followed by PA washout (duration dependent on the drug’s half-life) and subsequent Compound 1 titration for up to 16 days. Upon reaching the stable, well tolerated Compound 1 exposure, subjects continue on the same Compound 1 dosing regimen for 5 to 8 days; during this period subjects undergo oGTT, MMTT and GEBT tests. oGTTs and MMTTs are performed in the morning following an overnight fast of > 10 hours.

The titration phase (duration: up to 16 days) for Compound 1 subjects:
(50 mg in the evening + 12.5 mg in the morning) × 2 days (Dose level 1)
(50 mg in the evening + 25 mg in the morning) × 2 days (Dose level 2)
(50 mg in the evening + 37.5 mg in the morning) × 2 days (Dose level 3)
(50 mg in the evening + 50 mg in the morning) × 2 days (Dose level 4).

After 2 days of dosing at each dose level, including but not limited to, adverse events (AEs), vital signs and selected biomarkers are reviewed prior to dose escalation to the next level. Dose administered during the titration phase may be increased, maintained or decreased, based on the combination of outlined assessments. The investigator may consult with medical monitors and responsible physician for the guidance on titration decision.

If tolerating dose escalation well (escalation after 2 days at each level), titration is finished by the end of the 8th day (of dosing evening and morning) during the titration period and followed by 5 - 8 days of the same target exposure (Dose Level 4) in the Compound 1 Stable Dose Period for study assessment collection. Subjects who require slower titration (more than 2 days at 1 or more dose levels) have up to 16 days to reach the target exposure (Dose Level 4) or the subject’s individually highest tolerated dose level, followed by 5 to 8 days of the same dosing schedule in the Compound 1 Stable Dose Period. Subjects who do not tolerate dose increase to target exposure continue at their highest individually tolerated dose for the 5 - 8 days Compound 1 Stable Dose Period.

The 75 g oral glucose tolerance test (oGTT) is the reference method for diagnosing defined categories of glucose intolerance and Type 2 diabetes:

| | | | | | | | | |
|---|---|---|---|---|---|---|---|---|
| Assessments relative to meal start | -15 min | t = 0 | 10 min | 20 min | 30 min | 60 min | 90 min | 120 min |
| 75 g oral glucose solution | - | X | - | - | - | - | - | - |
| Plasma Glucose, Insulin, c-peptide^{a} | X | X^{b} | X | X | X | X | X | X |
| PK samples | X | X^{b} | X | X | X | X | X | X |

Abbreviations: PK = pharmacokinetics; t=0 denotes the first drink of glucose solution
^{a.}Additional biomarkers of metabolic effect are collected
^{b.} Samples are collected at t=0 immediately after oral glucose solution is consumed

Mixed meal tolerance test (MMTT) is a comprehensive physiological stimulus to insulin secretion as β-cells are responsive to certain amino acids and fatty acids in addition to glucose. Typically, MMTT is done with a liquid meal. Typical macronutrient composition is ~25 - 30% fat; ~15 - 20% protein; ~50 - 55% carbohydrates. Subjects are instructed to consume the entire meal within 10 minutes, but without undue haste. Immediately after the meal is consumed, 1500 mg of liquid acetaminophen is given for assessment of gastric emptying rate.

| | | | | | | | | | | | |
|---|---|---|---|---|---|---|---|---|---|---|---|
| Assessments relative to meal start | -15 min | t=0 | 10 min | 20 min | 30 min | 60 min | 90 min | 120 min | 180 min | 240 min | 300 min |
| Standard test meal breakfast | - | X | - | - | - | - | - | - | - | - | - |
| 1500 mg liquid Acetaminophen | - | X | - | - | - | - | - | - | - | - | - |
| Plasma Glucose, Insulin, C-peptide^{a} | X | X^{b} | X | X | X | X | X | X | X | X | X |
| Acetaminophen blood samples | X | X^{b} | X | X | X | X | X | X | X | X | X |
| VAS | X | - | - | - | - | X | X | X | X | X | X |
| PK samples | X | X^{b} - | X | X | X | X | X | X | X | X | X |

Abbreviations: PK = pharmacokinetics; t=0 denotes the first feeding of the standard meal test; VAS = visual analog scales of fullness, hunger, satiety, prospective food consumption.
a. Additional biomarkers of metabolic effect are collected (please see SOA below footnote n)
b. Samples are collected at t=0 immediately after first bite/drink.

Visual Analog Scale (VAS) of fullness, hunger, and satiety: The satiety, fullness, hunger, and prospective food consumption measures are self-administered 100 mm visual analog scales (VAS). The VAS has anchors labeled “not at all” (satisfied, full, hungry, interested, respectively) to “completely satisfied”, “totally full”, “never been more hungry”, and “a lot” respectively.

¹³C Spirulina Breath Test (GEBT): GEBT is performed in the following four steps: collection of pre-meal breath samples, consumption of served GEBT meal, collection of post-meal breath samples (45, 90, 120, 150, 180, 240 min) and submission of GEBT samples to the lab. GEBT test results are typically reported using the metric “kPCD.” At any measurement time t, kPCD(t) = 1000 X [Percent carbon-13 dose (PCD) in test meal excreted (as ¹³CO₂) per minute]. A larger kPCD value means a faster ¹³CO₂ excretion rate which is proportional to a faster rate of gastric emptying.

Positive and Negative Syndrome Scale (PANSS): PANSS scores are collected at screening, check-in and the last day of the Compound 1 Titration Period.

The PANSS is an interview-based measure of the severity of psychopathology in adults with psychotic disorders. The measure is comprised of 30 items and 3 scales: the Positive scale assesses hallucinations, delusions, and related symptoms; the Negative scale assesses emotional withdrawal, lack of motivation, and similar symptoms; and the General Psychopathology scale addresses other symptoms such as anxiety, somatic concern, and disorientation. An anchored Likert scale from 1 to 7, where values of 2 and above indicate the presence of progressively more severe symptoms, is used to score each item. Individual items are then summed to determine scores for the 3 scales, as well as a total score. A Composite scale score (Positive scale score minus Negative scale score) can also be calculated to show the relative valence of positive and negative symptoms. Total time required for the PANSS interview and scoring is approximately 30 40 minutes (Kay 1994, Opler 1992; Perkins 2000). PANSS raters will be required to meet specific training and education criteria before they are certified to rate for this study. PANSS requires input from an informant (e.g., caregiver, relative, friend, case worker).

Clinical Global Impressions - Severity Scale (CGI-S): CGI-S scores are collected at screening. The CGI-S is a clinician-rated assessment of the subject’s current illness state on a 7 point scale, where a higher score is associated with greater illness severity. Following a clinical interview, the CGI-S can be completed in 1 to 2 minutes. The CGI-S is administered by a qualified rater at the site.

Simpson-Angus Scale (SAS): SAS is collected at screening. The SAS is a clinician-rated assessment of neuroleptic-induced Parkinsonism consisting of 10 items. Items are anchor-based, rated on a 5 point scale of severity, and address rigidity, gait (bradykinesia), tremor, akathisia, shoulder shaking, glabellar tap, and salivation (Siddiqui 2009; Simpson 1970). The SAS is administered by a qualified rater at the site.

Barnes Akathisia Rating Scale (BARS): BARS is collected at screening. The BARS is a rating scale geared toward assessment of neuroleptic-induced akathisia, though it can be used to measure akathisia associated with other drugs as well. The BARS consists of four items, including one item assessing objective restlessness, two items targeting subjective restlessness (awareness and related distress), and one global clinical assessment item. All items are anchored and utilize a 4 point scale, except for the global rating which has a 6 point scale (from absence of akathisia through severe akathisia). The subjective and objective items are summed to yield a total score. The BARS can be administered in about 10 minutes (Barnes 1989; Barnes 2003). The BARS is administered by a qualified rater at the site.

Abnormal Involuntary Movement Scale (AIMS): AIMS is collected at screening. The AIMS is a clinician-rated assessment of abnormal movements consisting of unobtrusive observation of the subject at rest (with shoes removed) and several questions or instructions directed toward the subject. Using a severity scale ranging from 0 (none) to 4 (severe), clinicians rate dyskinesia in several body regions, including the facial area, extremities, and trunk. There are two items related to dental status, as well as three global impression items assessing overall severity, incapacitation, and the subject’s awareness of abnormal movements (Guy 1976; Munetz 1988). The AIMS raters are required to meet specific credential and educational criteria before they are certified to rate for this study. The AIMS is administered by a qualified rater at the site.

### Example 10: Food Effect on the Pharmacokinetics of Compound 1 Tablet Formulation in Healthy Human Subjects

Objective: The effect of food co-administration on the pharmacokinetics (PK) of tablet form Compound 1 was determined.

Methods: The study used an open-label randomized 2-period crossover design in 20 healthy volunteers. Subjects were fasted overnight then randomly assigned to 1 of 2 dosing sequences, A ➔ B or B ➔ A, where A was the fasted condition and B was the fed condition. For each period, a tablet of 50 mg Compound 1 was orally administered. The fed and fasted dosing periods were separated by one week. Serial plasma samples from each period were collected for PK analysis.

PK plasma samples were analyzed for Compound 1 concentration and its N-desmethyl (active) metabolite using validated LC-MS/MS methods. Solid phase extraction was used for sample cleanup. Stable isotope-labeled internal standards were used for corresponding analytes. Noncompartment PK parameters were calculated using Phoenix WinNonlin® software.

Results: The geometric mean Compound 1 Cₘₐₓ was 157.89 ng/mL under the fed condition and 157.95 ng/mL fasted. The geometric mean ratio of Cₘₐₓ was 99.96% with a 90% CI of 94.48 -105.77%. The geometric mean Compound 1 AUC_{0-∞} was 1584.2 ng*h/mL fed and 1589.2 ng*h/mL fasted. The geometric mean ratio for AUC_{0-∞} was 99.69% with a 90% CI of 95.02 -104.58%. There was a delay in tₘₐₓ (median difference of 1.47 h) in the fed condition compared to the fasted condition, likely caused by a delay in gastric emptying under the fed condition. For the metabolite, the Cₘₐₓ ratio of fed/fasted was 94.77% and the AUC_{0-∞} ratio was 98.29%.

Conclusions: A food effect study on the 50 mg tablet form of Compound 1 showed no difference in Compound 1 Cₘₐₓ and AUC_{0-∞} under fed or fasting conditions, with a slight increase in tₘₐₓ when administered with food, likely due to delayed gastric emptying during a meal. There was no change in the ratio of N-desmethyl active metabolite to parent Compound 1 under fed versus fasted dosing conditions.

The teachings of all patents, published applications and references cited herein are incorporated by reference in their entirety.

Various preferred embodiments [A] to [CZ] of the disclosure can be described in the text below:

[Embodiment A] A method of treating a patient having a metabolic disorder, comprising orally administering to the patient an effective amount of a compound, or a pharmaceutically acceptable salt thereof, or pharmaceutical composition disclosed herein.

[Embodiment B] A method of treating a patient having a metabolic disorder, comprising orally administering to the patient an effective amount of a compound of Formula I, or a pharmaceutically acceptable salt thereof, disclosed herein.

[Embodiment C] A method of treating a metabolic disorder described herein, comprising administering a compound disclosed herein in conjunction with one or more pharmaceutical agents.

[Embodiment D] A method of treating a patient having a metabolic disorder, comprising orally administering to the patient Compound 1

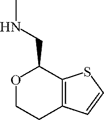

or a pharmaceutically acceptable salt thereof.

[Embodiment E] A method of any one of Embodiments [A] to [D] above, or according to other embodiments of the disclosure, wherein the metabolic disorder is Obesity; Overweightness or increased weight; Increased body mass index; Metabolic syndrome; Diabetes or diabetes-related disorders (Type 1 diabetes (insulin-dependent diabetes mellitus, IDDM) and Type 2 diabetes (non-insulin-dependent diabetes mellitus, NIDDM)); Diabetic complications; Impaired glucose tolerance; Elevated blood glucose; Insulin resistance; Insulin insensitivity; Hyperglycemia; Fatty liver disease; Non-alcoholic fatty liver disease; Hepatic insulin resistance; Glycosuria; Increased blood triglycerides; Increased appetite; or Dyslipidemia.

[Embodiment F] A method of any one of Embodiments [A] to [E] above, or according to other embodiments of the disclosure, wherein the metabolic disorder is associated with a neurological or psychiatric disease or disorder.

[Embodiment G] A method of Embodiment [F] above, or according to other embodiments of the disclosure, wherein the neurological or psychiatric disease or disorder is a Movement Disorder; Cognitive Disorders; Pain; Neurodevelopmental Disorder; Schizophrenia Spectrum and Other Psychotic Disorder; Bipolar and Related Disorder; Depressive Disorder; Anxiety Disorder; Obsessive-Compulsive and Related Disorder; Trauma- and Stressor-Related Disorder; Dissociative Disorder; Somatic Symptom and Related Disorder; Feeding and Eating Disorder; Elimination Disorder; Sleep-Wake Disorder; Sexual Dysfunction; Gender Dysphoria; Disruptive, Impulse-Control, and Conduct Disorder; Substance-Related and Addictive Disorder; Neurocognitive Disorder; Personality Disorder; Paraphilic Disorder; Other Mental Disorder; or Medication-Induced Movement Disorder and Other Adverse Effects of Medication.

[Embodiment H] A method of Embodiment [G] above, or according to other embodiments of the disclosure, wherein the Schizophrenia Spectrum and Other Psychotic Disorder is Schizotypal (Personality) Disorder; Delusional Disorder; Brief Psychotic Disorder; Shared Psychotic Disorder Schizophreniform Disorder; Schizophrenia (paranoid, disorganized, catatonic, or undifferentiated); Schizoaffective Disorder; Substance/Medication-Induced Psychotic Disorder; Psychotic Disorder Due to Another Medical Condition; Catatonia Associated With Another Mental Disorder (Catatonia Specifier); Catatonic Disorder Due to Another Medical Condition; Unspecified Catatonia; Other Specified Schizophrenia Spectrum and Other Psychotic Disorder; or Unspecified Schizophrenia Spectrum and Other Psychotic Disorder.

[Embodiment I] A method of any one of Embodiments [A] to [H] above, or according to other embodiments of the disclosure, wherein the compound, compound of Formula I, or Compound 1 is a pharmaceutically acceptable salt.

[Embodiment J] A method of Embodiments [D] to [I] above, or according to other embodiments of the disclosure, wherein the pharmaceutically acceptable salt of Compound 1 is HC1 salt.

[Embodiment K] A method of any one of Embodiments [D] to [J] above, or according to other embodiments of the disclosure, wherein Compound 1 is crystalline.

[Embodiment L] A method of any one of Embodiments [D] to [K] above, or according to other embodiments of the disclosure, comprising administering to the patient 10 mg to 250 mg of Compound 1, or a pharmaceutically acceptable salt thereof.

[Embodiment M] A method of any one of Embodiments [D] to [L] above, or according to other embodiments of the disclosure, comprising administering to the patient 25 mg to 100 mg of Compound 1, or a pharmaceutically acceptable salt thereof.

[Embodiment N] A method of any one of Embodiments [A] to [M] above, or according to other embodiments of the disclosure, comprising administering a therapeutically effective amount of Compound 1, or a pharmaceutically acceptable salt thereof, and a therapeutically effective amount of one or more other therapeutic agents.

[Embodiment O] A method of any one of Embodiments [A] to [N] above, or according to other embodiments of the disclosure, wherein the metabolic disorder is described in Chapter 5 of the International Statistical Classification of Diseases (ICD 11) coding system.

[Embodiment P] A method of any one of Embodiments [A] to [O] above, or according to other embodiments of the disclosure, wherein the metabolic disorder is described in Chapter 5 of the International Statistical Classification of Diseases (ICD 11) coding system as one of an endocrine, nutritional or metabolic disorder.

[Embodiment Q] A method of any one of Embodiments [F] to [P] above, or according to other embodiments of the disclosure, wherein the neurological or psychiatric diseases or disorders is described in the DSM-5, as amended or supplemented, or the International Statistical Classification of Diseases (ICD 11) coding system.

[Embodiment R] A method of any one of Embodiments [A] to [Q] above, or according to other embodiments of the disclosure, wherein the compounds and compositions disclosed herein may be used in combination with other therapies.

[Embodiment S] A method of any one of Embodiments [A] to [R] above, or according to other embodiments of the disclosure, wherein the other therapies are psychotherapy, cognitive behavioral therapy, electroconvulsive therapy, transcranial magnetic stimulation, vagus nerve stimulation, and deep-brain stimulation.

[Embodiment T] A method of treating schizophrenia in a human subject in need thereof comprising: (a) administering to said subject a therapeutically effective amount of Compound 1 at the subject’s bedtime (“evening dose”); and (b) administering to said subject a therapeutically effective amount of Compound 1 in the morning (“morning dose”).

[Embodiment U] A method of treating schizophrenia in a human subject in need thereof comprising: (a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and (b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL

[Embodiment V] A method of treating schizophrenia with weight reduction in a human subject in need thereof comprising: (a) administering to said subject a therapeutically effective amount of Compound 1 at the subject’s bedtime (“evening dose”); and (b) administering to said subject a therapeutically effective amount of Compound 1 in the morning (“morning dose”), and the subject’s weight is reduced after about 4-52 weeks of Compound 1 administration.

[Embodiment W] A method of treating schizophrenia with weight reduction in a human subject in need thereof comprising: (a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and (b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL, and the subject’s weight is reduced after about 4-52 weeks of Compound 1 administration.

[Embodiment X] A method of treating schizophrenia and improving insulin secretion efficiency in a type 2 diabetic human subject in need thereof comprising: (a) administering to said subject a therapeutically effective amount of Compound 1 at the subject’s bedtime (“evening dose”); and (b) administering to said subject a therapeutically effective amount of Compound 1 in the morning (“morning dose”), and improving the subject’s insulin secretion efficiency.

[Embodiment Y] A method of treating schizophrenia and improving insulin secretion efficiency in a type 2 diabetic human subject in need thereof comprising: (a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and (b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL, and improving the subject’s insulin secretion efficiency.

[Embodiment Z] A method of treating adjunctive major depressive disorder (aMDD) in a human subject in need thereof comprising: (a) administering to said subject a therapeutically effective amount of Compound 1 at the subject’s bedtime (“evening dose”); and (b) administering to said subject a therapeutically effective amount of Compound 1 in the morning (“morning dose”).

[Embodiment AA ] A method of treating adjunctive major depressive disorder (aMDD) in a human subject in need thereof comprising: (a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and (b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL.

[Embodiment AB ] A method of treating generalized anxiety disorder (GAD) in a human subject in need thereof comprising: (a) administering to said subject a therapeutically effective amount of Compound 1 at the subject’s bedtime (“evening dose”); and (b) administering to said subject a therapeutically effective amount of Compound 1 in the morning (“morning dose”).

[Embodiment AC] A method of treating generalized anxiety disorder (GAD) in a human subject in need thereof comprising: (a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and (b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL.

[Embodiment AD] A method of reducing weight in a human subject in need thereof comprising: (a) administering to said subject a therapeutically effective amount of Compound 1 at the subject’s bedtime (“evening dose”); and (b) administering to said subject a therapeutically effective amount of Compound 1 in the morning (“morning dose”), and the subject’s weight is reduced after about 4-52 weeks of Compound 1 administration.

[Embodiment AE] A method of reducing weight in a human subject in need thereof comprising: (a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and (b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL, and the subject’s weight is reduced after about 4-52 weeks of Compound 1 administration.

[Embodiment AF] A method of treating type 2 diabetes in a human subject in need thereof comprising: (a) administering to said subject a therapeutically effective amount of Compound 1 at the subject’s bedtime (“evening dose”); and (b) administering to said subject a therapeutically effective amount of Compound 1 in the morning (“morning dose”), to improve efficiency of insulin secretion.

[Embodiment AG] A method of treating type 2 diabetes in a human subject in need thereof comprising: (a) administering to said subject Compound 1 at the subject’s bedtime (“evening dose”) in an amount sufficient to provide an average peak plasma concentration in a range of about 150 ng/mL to about 400 ng/mL and an average trough plasma concentration in a range of about 25 ng/mL to about 200 ng/mL; and (b) administering to said subject Compound 1 in the morning (“morning dose”) in an amount sufficient to provide an average peak plasma concentration of in a range of about 150 ng/mL to about 400 ng/mL and an average trough plasma concentration in a range of about 25 ng/mL to about 200 ng/mL, to improve efficiency of insulin secretion.

[Embodiment AH] A method of treating schizophrenia, depression, or anxiety in a human subject in need thereof comprising: a) administering to the subject a therapeutically effective amount of Compound 1 at the subject’s bedtime (“evening dose”); and b) administering to the subject a therapeutically effective amount of Compound 1 in the morning (“morning dose”).

[Embodiment AI] A method of treating schizophrenia, depression, or anxiety with weight reduction in a human subject in need thereof comprising: a) administering to the subject a therapeutically effective amount of Compound 1 at the subject’s bedtime (“evening dose”); and b) administering to the subject a therapeutically effective amount of Compound 1 in the morning (“morning dose”), and the subject’s weight is reduced after about 4-52 weeks of Compound 1 administration.

[Embodiment AJ] A method of treating schizophrenia, depression, or anxiety and improving insulin secretion efficiency in a type 2 diabetic human subject in need thereof comprising: a) administering to the subject a therapeutically effective amount of Compound 1 at the subject’s bedtime (“evening dose”); and b) administering to the subject a therapeutically effective amount of Compound 1 in the morning (“morning dose”), and improving the subject’s insulin secretion efficiency.

[Embodiment AK] A method of treating schizophrenia, depression, or anxiety in a human subject in need thereof having an excess body mass index (BMI) defined by a BMI ≥ 25 comprising administering to the subject a therapeutically effective amount of Compound 1.

[Embodiment AL] A method of treating schizophrenia, depression, or anxiety in a human subject in need thereof having diabetes or metabolic syndrome comprising administering to the subject a therapeutically effective amount of Compound 1.

[Embodiment AM] A method of treating schizophrenia, depression, or anxiety in a human subject in need thereof having a metabolic disorder selected from obesity; overweightness or increased weight; increased body mass index; metabolic syndrome; diabetes or diabetes-related disorders (type 1 diabetes (insulin-dependent diabetes mellitus, IDDM) and Type 2 diabetes (non-insulin-dependent diabetes mellitus, NIDDM)); diabetic complications; impaired glucose tolerance; elevated blood glucose; insulin resistance; insulin insensitivity; hyperglycemia; fatty liver disease; non-alcoholic fatty liver disease; hepatic insulin resistance; glycosuria; increased blood triglycerides; increased appetite; or dyslipidemia, comprising administering to the subject a therapeutically effective amount of Compound 1.

[Embodiment AN] A method of treating schizophrenia, depression, or anxiety in a human subject in need thereof having a metabolic disorder selected from insulin resistance, insulin insensitivity, impaired glucose tolerance, and elevated blood glucose comprising administering to the subject a therapeutically effective amount of Compound 1.

[Embodiment AO] A method of treating schizophrenia, depression, or anxiety in a human subject in need thereof, wherein the human subject is further in need of delayed gastric emptying, comprising administering to the subject a therapeutically effective amount of Compound 1.

[Embodiment AP] A method of treating schizophrenia, depression, or anxiety in a human subject in need thereof, comprising administering to the subject a therapeutically effective amount of Compound 1 in the fed state.

[Embodiment AQ] A method of any one of Embodiments [T] to [AJ] above, or according to other embodiments of the disclosure, wherein the morning dose is from about 12.5 mg to 125 mg.

[Embodiment AR] A method of any one of Embodiments [T] to [AJ] above, or according to other embodiments of the disclosure, wherein the evening dose is from about 12.5 mg to 125 mg.

[Embodiment AS] A method of any one of Embodiments [T] to [AJ] above, or according to other embodiments of the disclosure, wherein the morning dose is administered up to 4 hours prior to or with a meal.

[Embodiment AT] A method of any one of Embodiments [T] to [AJ] above, or according to other embodiments of the disclosure, wherein the evening dose is administered up to 4 hours prior to or with a meal.

[Embodiment AU] A method of any one of Embodiments [T] to [AJ] above, or according to other embodiments of the disclosure, wherein each of the morning dose and evening dose is administered up to 4 hours prior to or with a meal.

[Embodiment AV] A method of any one of Embodiments [T] to [AJ] above, or according to other embodiments of the disclosure, wherein each of the morning dose and evening dose is administered with a meal.

[Embodiment AW] A method of any of Embodiments [AH] to [AP] above, or according to other embodiments of the disclosure, wherein Compound 1 is administered once daily.

[Embodiment AX] A method of any of Embodiments [T] to [AW] above, or according to other embodiments of the disclosure, wherein the subject has an excess BMI defined by a BMI ≥ 25, 27.5, or 30 kg/m².

[Embodiment AY] A method of any of Embodiments [T] to [AX] above, or according to other embodiments of the disclosure, wherein the subject has an excess BMI defined by a BMI ≥ 25, 27.5, or 30 kg/m² and a history of BMI < 25 kg/m² prior to schizophrenia, depression, or anxiety diagnosis.

[Embodiment AZ] A method any of Embodiments [T] to [AY] above, or according to other embodiments of the disclosure, wherein the subject is on a typical or atypical antipsychotic.

[Embodiment BA] A method of any of Embodiments [T] to [AZ] above, or according to other embodiments of the disclosure, wherein the subject is on risperidone, olanzapine, quetiapine or aripiprazole prior to commencing therapy with Compound 1.

[Embodiment BB] A method of any of Embodiments [T] to [BA] above, or according to other embodiments of the disclosure, wherein the subject has an excess BMI defined by a BMI ≥ 30 kg/m² and a history of BMI < 25 kg/m² prior to schizophrenia, depression, or anxiety diagnosis, and the subject is on a typical or atypical antipsychotic.

[Embodiment BC] A method of any of Embodiments [T] to [BB] above, or according to other embodiments of the disclosure, wherein the subject has an excess BMI induced by a typical or atypical antipsychotic.

[Embodiment BD] A method of any of Embodiments [T] to [BC] above, or according to other embodiments of the disclosure, wherein the subject has diabetes.

[Embodiment BE] A method of any of Embodiments [T] to [BD] above, or according to other embodiments of the disclosure, wherein the subject is on medication for diabetes.

[Embodiment BF] A method of any of Embodiments [T] to [BE] above, or according to other embodiments of the disclosure, wherein the subject has metabolic syndrome.

[Embodiment BG] A method of any of Embodiments [T] to [BF] above, or according to other embodiments of the disclosure, wherein the subject is on medication for metabolic syndrome.

[Embodiment BH] A method of any of Embodiments [T] to [AL] and [AN] to [BG] above, or according to other embodiments of the disclosure, wherein the subject has a metabolic disorder selected from obesity; overweightness or increased weight; increased body mass index; metabolic syndrome; diabetes or diabetes-related disorders (type 1 diabetes (insulin-dependent diabetes mellitus, IDDM) and Type 2 diabetes (non-insulin-dependent diabetes mellitus, NIDDM)); diabetic complications; impaired glucose tolerance; elevated blood glucose; insulin resistance; insulin insensitivity; hyperglycemia; fatty liver disease; non-alcoholic fatty liver disease; hepatic insulin resistance; glycosuria; increased blood triglycerides; increased appetite; or dyslipidemia.

[Embodiment BI] A method of any of Embodiments [T] to [BH] above, or according to other embodiments of the disclosure, wherein the subject has insulin resistance.

[Embodiment BJ] A method of any of Embodiments [T] to [BI] above, or according to other embodiments of the disclosure, wherein the subject is on medication for insulin resistance.

[Embodiment BK] A method of any of Embodiments [T] to [BJ] above, or according to other embodiments of the disclosure, wherein the subject has insulin insensitivity.

[Embodiment BL] A method of any of Embodiments [T] to [BK] above, or according to other embodiments of the disclosure, wherein the subject has insulin sensitivity based on the glucose rate of appearance, glucose rate of disappearance, and/or glycerol rate of appearance.

[Embodiment BM] A method of any of Embodiments [T] to [BL] above, or according to other embodiments of the disclosure, wherein the subject is on medication for insulin insensitivity.

[Embodiment BN] A method of any of Embodiments [T] to [BM] above, or according to other embodiments of the disclosure, wherein the subject has impaired glucose tolerance.

[Embodiment BO] A method of any of Embodiments [T] to [BN] above, or according to other embodiments of the disclosure, wherein the subject has impaired glucose tolerance based on the patient’s insulin, C-peptide, and/or glucose response to feeding.

[Embodiment BP] A method of any of Embodiments [T] to [BO] above, or according to other embodiments of the disclosure, wherein the subject has impaired glucose tolerance based on a multiplex of metabolic biomarkers selected from the group consisting of glucose, c peptide, insulin, glucagon, leptin, GLP-1, and combinations thereof.

[Embodiment BQ] A method of any of Embodiments [T] to [BP] above, or according to other embodiments of the disclosure, wherein subject has impaired glucose tolerance based on markers of β cell function and incretins.

[Embodiment BR] A method of any of Embodiments [T] to [BQ] above, or according to other embodiments of the disclosure, wherein the subject is on medication for impaired glucose tolerance.

[Embodiment BS] A method of any of Embodiments [T] to [BR] above, or according to other embodiments of the disclosure, wherein the subject has elevated blood glucose.

[Embodiment BT] A method of any of Embodiments [T] to [BS] above, or according to other embodiments of the disclosure, wherein the subject is on medication for elevated blood glucose.

[Embodiment BU] A method of any of Embodiments [T] to [BT] above, or according to other embodiments of the disclosure, wherein the subject is in need of delayed gastric emptying based on the subject’s fullness, hunger and/or satiety, following a meal.

[Embodiment BV] A method of any of Embodiments [T] to [BU] above, or according to other embodiments of the disclosure, wherein the subject is in need of delayed gastric emptying based on an increase in BMI subsequent to schizophrenia diagnosis or commencement of antipsychotic therapy.

[Embodiment BW] A method of any of Embodiments [T] to [BV] above, or according to other embodiments of the disclosure, wherein the subject is in need of delayed gastric emptying based on a ¹³C Spirulina Breath Test (GEBT).

[Embodiment BX] A method of any of Embodiments [T] to [BW] above, or according to other embodiments of the disclosure, wherein Compound 1 is administered in the fed state.

[Embodiment BY] A method of any of Embodiments [T] to [BX] above, or according to other embodiments of the disclosure, wherein the subject is in need of treatment for schizophrenia.

[Embodiment BZ] A method of any of Embodiments [T] to [BY] above, or according to other embodiments of the disclosure, wherein the subject is in need of treatment for schizophrenia, and the subject has a CGI-S score ≤ 4 (normal to moderately ill), a SAS score < 2, an AIMS score < 3, and a BARS score < 3.

[Embodiment CA] A method of any of Embodiments [T] to [BZ] above, or according to other embodiments of the disclosure, wherein the subject is in need of treatment for schizophrenia, and the subject has a positive and negative symptom scale (PANSS) total score ≤ 80.

[Embodiment CB] A method of any of Embodiments [T] to [CA] above, or according to other embodiments of the disclosure, wherein the subject is in need of treatment for depression.

[Embodiment CC] A method of any of Embodiments [T] to [CB] above, or according to other embodiments of the disclosure, wherein the subject is in need of treatment for aMDD.

[Embodiment CD] A method of any of Embodiments [T] to [CC] above, or according to other embodiments of the disclosure, wherein the subject is in need of treatment for anxiety.

[Embodiment CE] A method of any of Embodiments [T] to [CD] above, or according to other embodiments of the disclosure, wherein the subject is in need of treatment for GAD.

[Embodiment CF] A method of any one of Embodiments [T] to [CE] above, or according to other embodiments of the disclosure, wherein the subject has waist circumference ≥ 40 inches (101.6 cm) for men or ≥ 35 inches (88.9 cm) for women.

[Embodiment CG] A method of any one of Embodiments [T] to [CF] above, or according to other embodiments of the disclosure, wherein the subject has triglyceride concentration ≥ 150 mg/dL (1.69 mmol/L) or ongoing treatment with triglyceride lowering medication.

[Embodiment CH] A method of any one of Embodiments [T] to [CG] above, or according to other embodiments of the disclosure, wherein the subject has (i) HDL-cholesterol concentration < 40 mg/dL (1.03 mmol/L) for men or < 50 mg/dL (1.29 mmol/L) for women or (ii) ongoing treatment with cholesterol lowering medication.

[Embodiment CI] A method of any one of Embodiments [T] to [CH] above, or according to other embodiments of the disclosure, wherein the subject has fasting glucose concentrations ≥ 100 mg/dL (5.6 mmol/L).

[Embodiment CJ] A method of any one of Embodiments [T] to [CI] above, or according to other embodiments of the disclosure, wherein the subject has (i) standing or supine blood pressure ≥ 130/85 mmHg or (ii) ongoing treatment with antihypertensive medication.

[Embodiment CK] A method of any one of Embodiments [T] to [CJ] above, or according to other embodiments of the disclosure, wherein the subject has three, four or five of the following criteria (a) - (e): (a) waist circumference ≥ 40 inches (101.6 cm) for men or ≥ 35 inches (88.9 cm) for women; (b) triglyceride concentration ≥ 150 mg/dL (1.69 mmol/L) or ongoing treatment with triglyceride lowering medication; (c) (i) HDL-cholesterol concentration < 40 mg/dL (1.03 mmol/L) for men or < 50 mg/dL (1.29 mmol/L) for women or (ii) ongoing treatment with cholesterol lowering medication; (d) fasting glucose concentrations ≥ 100 mg/dL (5.6 mmol/L); and (e) (i) standing or supine blood pressure ≥ 130/85 mmHg or (ii) ongoing treatment with antihypertensive medication.

[Embodiment CL] A method of any one of Embodiments [T] to [CK] above, or according to other embodiments of the disclosure, wherein the subject has glycemic derived insulin resistance as evidenced by 5.7% ≤ HbAlc ≤ 6.4%.

[Embodiment CM] A method of any one of Embodiments [T] to [CL] above, or according to other embodiments of the disclosure, wherein the subject has glycemic derived insulin resistance as evidenced by fasting HOMA-IR ([Insulin uIU/ml x Glucose mg/dl] / 405) ≥ 2.22.

[Embodiment CN] A method of any one of Embodiments [T] to [CM] above, or according to other embodiments of the disclosure, wherein the subject has three, four or five of the following criteria (a) - (e): (a) waist circumference ≥ 40 inches (101.6 cm) for men or ≥ 35 inches (88.9 cm) for women; (b) triglyceride concentration ≥ 150 mg/dL (1.69 mmol/L) or ongoing treatment with triglyceride lowering medication; (c) (i) HDL-cholesterol concentration < 40 mg/dL (1.03 mmol/L) for men or < 50 mg/dL (1.29 mmol/L) for women or (ii) ongoing treatment with cholesterol lowering medication; (d) fasting glucose concentrations ≥ 100 mg/dL (5.6 mmol/L); and (e) (i) standing or supine blood pressure ≥ 130/85 mmHg or (ii) ongoing treatment with antihypertensive medication; and further wherein the subject has glycemic derived insulin resistance as evidenced by 5.7%≤ HbAlc ≤ 6.4% or fasting HOMA-IR ([Insulin uIU/ml x Glucose mg/dl] / 405) ≥ 2.22.

[Embodiment CO] A method of any one of Embodiments [T] to [CN] above, or according to other embodiments of the disclosure, wherein the Cₘₐₓ and AUC_{0-∞} ratios for Compound 1 and the N-desmethyl metabolite of Compound 1 in the fed and fasted states is greater than 90 or greater than 95%.

[Embodiment CP] A method of any of Embodiments [AK] to [AP] and [AW] to [CO] above, or according to other embodiments of the disclosure, wherein the daily dose of Compound 1 is from about 12.5 mg to about 150 mg.

[Embodiment CQ] A method of any of Embodiments [AK] to [AP] and [AW] to [CO] above, or according to other embodiments of the disclosure, wherein the daily dose of Compound 1 is from about 25 mg to about 100 mg.

[Embodiment CR] A method of any of Embodiments [AK] to [AP] and [AW] to [CO] above, or according to other embodiments of the disclosure, wherein the daily dose of Compound 1 is about 12.5, 25, 37.5, 50, 62.5, 75, 87.5, 100, 112.5, or 125 mg.

[Embodiment CS] A method of any one of Embodiments [T] to [CR] above, or according to other embodiments of the disclosure, wherein the subject’s glycemic control is improved as compared to when Compound 1 is not administered.

[Embodiment CT] A method of any one of Embodiments [T] to [CS] above, or according to other embodiments of the disclosure, wherein the subject’s satiety is increased as compared to when Compound 1 is not administered.

[Embodiment CU] A method of any one of Embodiments [T] to [CT] above, or according to other embodiments of the disclosure, wherein the subject’s appetite is reduced as compared to when Compound 1 is not administered.

[Embodiment CV] A method of any one of Embodiments [T] to [CU] above, or according to other embodiments of the disclosure, wherein the subject’s glucose excursion is reduced as compared to when Compound 1 is not administered.

[Embodiment CW] A method of any one of Embodiments [T] to [CV] above, or according to other embodiments of the disclosure, wherein the subject’s insulin uptake is improved as compared to when Compound 1 is not administered.

[Embodiment CX] A method of any one of Embodiments [T] to [CW] above, or according to other embodiments of the disclosure, wherein the subject’s body fat is reduced as compared to when Compound 1 is not administered.

[Embodiment CY] A method of any one of Embodiments [T] to [CX] above, or according to other embodiments of the disclosure, wherein Compound 1 is a pharmaceutically acceptable salt.

[Embodiment CZ] A method of any one of Embodiments [T] to [CY] above, or according to other embodiments of the disclosure, wherein the pharmaceutically acceptable salt of Compound 1 is a hydrochloride salt.

[Embodiment DA] A method of any one of Embodiments [T] to [CZ] above, or according to other embodiments of the disclosure, wherein Compound 1 is crystalline.

[Embodiment DB] A method of any of Embodiments [T] to [DA] above, or according to other embodiments of the disclosure, wherein Compound 1 is a crystalline hydrochloride salt.

[Embodiment DC] A method of any one of Embodiments [T] to [DB] above, or according to other embodiments of the disclosure, comprising administering a therapeutically effective amount of Compound 1, or a pharmaceutically acceptable salt thereof, and a therapeutically effective amount of one or more other therapeutic agents.

While example embodiments have been particularly shown and described, it will be understood by those skilled in the art that various changes in form and details may be made therein without departing from the scope of the embodiments encompassed by the appended claims.

1-3. (canceled)

4. A method of treating schizophrenia, depression, bipolar disorder, or anxiety in a human subject having a body mass index (BMI) of at least 25 and reducing the subject’s BMI comprising administering to the subject a therapeutically effective amount of Compound 1.

5. (canceled)

6. A method of treating schizophrenia, depression, bipolar disorder, or anxiety in a human subject in need thereof having a metabolic disorder selected from obesity; overweightness or increased weight; increased body mass index; metabolic syndrome; diabetes or diabetes-related disorders (type 1 diabetes (insulin-dependent diabetes mellitus, IDDM) and Type 2 diabetes (non-insulin-dependent diabetes mellitus, NIDDM)); diabetic complications; impaired glucose tolerance; elevated blood glucose; insulin resistance; insulin insensitivity; hyperglycemia; fatty liver disease; non-alcoholic fatty liver disease; hepatic insulin resistance; glycosuria; increased blood triglycerides; increased appetite; or dyslipidemia, comprising administering to the subject a therapeutically effective amount of Compound 1.

7-8. (canceled)

9. A method of treating schizophrenia, depression, bipolar disorder, or anxiety in a human subject in need thereof, comprising administering to the human subject a therapeutically effective amount of Compound 1 in the fed state.

10-12. (canceled)

13. The method of claim 4, wherein Compound 1 is administered once daily.

14. The method of claim 4, wherein the subject has an excess BMI defined by a BMI ≥ 25, 27.5, or 30 kg/m2.

15. (canceled)

16. The method of claim 4, wherein the subject is on a typical or atypical antipsychotic prior to commencing therapy with Compound 1.

17. (canceled)

18. The method of claim 4, wherein the subject has an excess BMI defined by a BMI ≥ 30 kg/m2 and a history of BMI < 25 kg/m² prior to schizophrenia, depression, or anxiety diagnosis, and the subject is on a typical or atypical antipsychotic.

19. The method of claim 4, wherein the subject has an excess BMI induced by a typical or atypical antipsychotic.

20-23. (canceled)

24. The method of claim 4, wherein the subject has a metabolic disorder selected from obesity; overweightness or increased weight; increased body mass index; metabolic syndrome; diabetes or diabetes-related disorders (type 1 diabetes (insulin-dependent diabetes mellitus, IDDM) and Type 2 diabetes (non-insulin-dependent diabetes mellitus, NIDDM)); diabetic complications; impaired glucose tolerance; elevated blood glucose; insulin resistance; insulin insensitivity; hyperglycemia; fatty liver disease; non-alcoholic fatty liver disease; hepatic insulin resistance; glycosuria; increased blood triglycerides; increased appetite; or dyslipidemia.

25-39. (canceled)

40. The method of claim 4, wherein Compound 1 is administered in the fed state.

41-52. (canceled)

53. The method of claim 4, wherein the subject has at least three of the following:
a) waist circumference ≥ 40 inches (101.6 cm) for men or ≥ 35 inches (88.9 cm) for women;
b) triglyceride concentration ≥ 150 mg/dL (1.69 mmol/L) or ongoing treatment with triglyceride lowering medication;
c) (i) HDL-cholesterol concentration < 40 mg/dL (1.03 mmol/L) for men or < 50 mg/dL (1.29 mmol/L) for women or (ii) ongoing treatment with cholesterol lowering medication;
d) fasting glucose concentrations ≥ 100 mg/dL (5.6 mmol/L); and
e) (i) standing or supine blood pressure ≥ 130/85 mmHg or (ii) ongoing treatment with antihypertensive medication.

54-58. (canceled)

59. The method of claim 4, wherein the daily dose of Compound 1 or pharmaceutically acceptable salt thereof is from about 25 mg to about 100 mg.

60. The method of claim 4, wherein the daily dose of Compound 1 or pharmaceutically acceptable salt thereof is about 12.5, 25, 37.5, 50, 62.5, 75, 87.5, 100, 112.5, or 125 mg.

61. The method of claim 4, wherein the method is further undertaken to improve the subject’s glycemic control, further comprising improving the subject’s glycemic control.

62-64. (canceled)

65. The method of claim 4, wherein the method is further undertaken to improve the subject’s insulin uptake, further comprising improving the subject’s insulin uptake.

66. The method of claim 4, wherein the method is further undertaken to reduce the subject’s body fat, further comprising reducing the subject’s body fat.

67. The method of claim 4, wherein Compound 1 is a pharmaceutically acceptable salt.

68. The method of claim 4, wherein Compound 1 is a hydrochloride salt.

69. (canceled)
